(19) United States
(12) Patent Application Publication
(10) Pub. No.: US 2023/0124669 A1
(43) Pub. Date: Apr. 20, 2023

(54) HETERODIMERIC PROTEINS WITH FC MUTATIONS

(71) Applicant: Adagene AG, Basel (CH)

(72) Inventors: Peter Peizhi LUO, Lansdale, PA (US); Fangyong DU, New Haven, CT (US); Guizhong LIU, Suzhou (CN); Zhengxi DAI, Suzhou, Jiangsu (CN); Jianfeng SHI, Suzhou, Jiangsu (CN); Zhixiong LIN, Suzhou, Jiangsu (CN); Yan LI, Suzhou, Jiangsu (CN)

(73) Assignees: Peter Peizhi Luo, Suzhou, Jiangsu (CH); Adagene AG, Basel (CH)

(21) Appl. No.: 17/759,282

(22) PCT Filed: Jan. 22, 2021
(86) PCT No.: PCT/CN2021/073347

§ 371 Date: Jul. 21, 2022

(30) Foreign Application Priority Data
Jan. 23, 2020 (CN) PCT/CN2020/073960

(51) Int. Cl. C07K 16/28; C07K 16/32; A61P 35/00; A61P 11/00

(52) U.S. Cl. CPC C07K 16/2827; C07K 16/2878; C07K 16/2818; C07K 16/2809; C07K 16/32; A61P 35/00; A61P 11/00; C07K 2317/526; C07K 2317/21; C07K 2317/53; C07K 2317/52; C07K 2317/31; C07K 2317/622; C07K 2317/14; C07K 2317/51; C07K 2317/515; C07K 2317/522; C07K 2317/524; C07K 2317/565; C07K 2317/55; C07K 2317/33; A61K 2039/505

(57) ABSTRACT

Provided are heterodimeric proteins comprising polypeptides having CH3 domains with engineered residues that form disulfide bonds and/or salt bridges. Also provided are activatable antibodies targeting CD3 and/or HER2. Compositions, methods of manufacture and methods of treatment using the heterodimeric proteins and the activatable antibodies are further provided.

### CROSS REFERENCE TO RELATED APPLICATIONS

This application claims the priority benefit of International Application No. PCT/CN2020/073960, filed on Jan. 23, 2020, which is incorporated herein by reference in its entirety.

### FIELD OF THE INVENTION

The present application relates to heterodimeric proteins (such as bispecific antibodies) and activatable antibodies, methods of preparation, and methods of use thereof.

### REFERENCE TO SEQUENCE LISTING

The content of the following submission on ASCII text file is incorporated herein by reference in its entirety: a computer readable form (CRF) of the Sequence Listing (file name: 695402001041SEQLIST.txt, date recorded: Jan. 21, 2020, size: 656 KB).

### BACKGROUND OF THE INVENTION

Multispecific antibodies can simultaneously bind multiple different antigens. This property enables the development of therapeutic strategies that are not possible with conventional monoclonal antibodies. One format of multispecific antibodies are heterodimeric proteins, e.g., antibodies made up of separate chains that bind different antigens. Such heterodimeric, multispecific antibodies can only target multiple antigens correctly when assembled with the right complement of monomer components. Therefore, there exists a need in the art for multispecific antibodies that heterodimerize in a specific and stable manner.

An activatable antibody exhibits an “activatable” conformation such that an antigen-binding moiety contained therein is less accessible to bind to its target when uncleaved than after cleavage in the presence of one or more specific proteases. Activatable antibodies thus provide antigen-specific binding proteins that are only capable of binding their targets in certain contexts (e.g., in the protease-rich tumor microenvironment). Bispecific T cell engagers are bispecific antibodies (BiTE) that are capable of binding to both T cells and target cells such as tumor cells. Because of their on-target off-tumor effects, BiTE molecules are associated with high cytotoxicity, including toxicity to the central nervous system (CNS) and cytokine storm. There is a need for activatable BiTE molecules with enhanced specificity and reduced side effects.

All references cited herein, including patent applications, patent publications, non-patent literature, and UniProtKB/Swiss-Prot/GenBank Accession numbers are herein incorporated by reference in their entirety, as if each individual reference were specifically and individually indicated to be incorporated by reference.

### BRIEF SUMMARY OF THE INVENTION

The present application provides heterodimeric proteins comprising CH3 domains having engineered residues that form disulfide bonds and/or salt bridges. Also provided are activatable antibodies targeting CD3 and/or HER2.

Accordingly, one aspect of the present application provides a heterodimeric protein comprising a first polypeptide comprising a first immunoglobulin heavy chain constant domain 3 (CH3 domain) and a second polypeptide comprising a second CH3 domain, wherein: i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and wherein the amino acid residue numbering is based on EU numbering. In some embodiments, i) the first CH3 domain comprises N390C substitution and the second CH3 domain comprises S400C substitution, or the first CH3 domain comprises S400C substitution and the second CH3 domain comprises N390C substitution; or ii) the first CH3 domain comprises K392C substitution and the second CH3 domain comprises V397C substitution, or the first CH3 domain comprises V397C substitution and the second CH3 domain comprises K392C substitution; or iii) the first CH3 domain comprises K392C substitution and the second CH3 domain comprises S400C substitution, or the first CH3 domain comprises S400C substitution and the second CH3 domain comprises K392C substitution.

In some embodiments according to any one of the heterodimeric proteins described above, i) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or ii) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or iii) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of i) and ii), or a combination of i) and iii), and wherein the amino acid residue numbering is based on EU numbering. In some embodiments, the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356; and wherein the amino acid residue numbering is based on EU numbering. In some embodiments, i) the positively charged residue is a lysine (K) residue, and the negatively charged residue is an aspartic acid (D) residue; or ii) the positively charged residue is a lysine (K) residue, and the negatively charged residue is a glutamic acid (E) residue; or iii) the positively charged residue is an arginine (R) residue, and the negatively charged residue is an aspartic acid (D) residue; or iv) the positively charged residue is an arginine (R) residue, and the negatively charged residue is a glutamic acid (E) residue. In some embodiments, i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or ii) the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions; or iii) the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions.

In some embodiments according to any one of the heterodimeric proteins described above, i) the first CH3 domain further comprises K392D and K409D substitutions and the second CH3 domain further comprises D356K, and D399K substitutions, or the first CH3 domain further comprises D356K and D399K substitutions and the second CH3 domain further comprises K392D and K409D substitutions; or ii) the first CH3 domain further comprises L368D and K370S substitutions and the second CH3 domain further comprises E357Q and S364K substitutions, or the first CH3 domain further comprises E357Q and S364K substitutions and the second CH3 domain further comprises L368D and K370S substitutions; or iii) the first CH3 domain further comprises L351K and T366K substitutions and the second CH3 domain further comprises L351D and L368E substitutions, or the first CH3 domain further comprises L351D and L368E substitutions and the second CH3 domain further comprises L351K and T366K substitutions; or (iv) the first CH3 domain further comprises P395K. P396K and V397K substitutions and the second CH3 domain comprises T394D, P395D and P3% D substitutions, or the first CH3 domain further comprises T394D, P395D and P3% D substitutions and the second CH3 domain further comprises P395K, P396K and V397K substitutions; or (v) the first CH3 domain further comprises F405E, Y407E and K409E substitutions and the second CH3 domain comprises F405K and Y407K substitutions, or the first CH3 domain further comprises F405K and Y407K substitutions and the second CH3 domain further comprises F405E, Y407E and K409E substitutions.

In some embodiments according to any one of the heterodimeric proteins described above, i) the first CH3 domain comprises E357K, S364K and N390C substitutions and the second CH3 domain comprises L351D, K370D, and S400C substitutions, or the first CH3 domain comprises L351D, K370D, and S400C substitutions and the second CH3 domain comprises E357K, S364K and N390C substitutions; or ii) the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions; or iii) the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions; or iv) the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions.

In some embodiments according to any one of the heterodimeric proteins described above, the first CH3 domain and the second CH3 domain further comprise knob-into-hole residues. In some embodiments, i) the first CH3 domain comprises T336S, L368A and Y407V substitutions and the second CH3 domain comprises T366W substitution, or the first CH3 domain comprises T366W substitution and the second CH3 domain comprises T336S, L368A and Y407V substitutions; or ii) the first CH3 domain comprises L368V and Y407V substitutions and the second CH3 domain comprises T366W substitution, or the first CH3 domain comprises T366W substitution and the second CH3 domain comprises L368V and Y407V substitutions.

Another aspect of the present application provides a heterodimeric protein comprising a first polypeptide comprising a first CH3 domain and a second polypeptide comprising a second CH3 domain, wherein: i) the first CH3 domain comprises a positively charged residue at position 357 and the second CH3 domain comprises a negatively charged residue at position 351, or the first CH3 domain comprises a negatively charged residue at position 351 and the second CH3 domain comprises a positively charged residue at position 357; or ii) the first CH3 domain comprises a positively charged residue at position 411 and the second CH3 domain comprises a negatively charged residue at position 370, or the first CH3 domain comprises a negatively charged residue at position 370 and the second CH3 domain comprises a positively charged residue at position 411; or iii) the first CH3 domain comprises a positively charged residue at position 364 and the second CH3 domain comprises a negatively charged residue at position 370, or the first CH3 domain comprises a negatively charged residue at position 370 and the second CH3 domain comprises a positively charged residue at position 364; and wherein the amino acid residue numbering is based on EU numbering. In some embodiments, the first CH3 domain comprises a positively charged residue at position 356 and the second CH3 domain comprises a negatively charged residue at position 439, or first CH3 domain comprises a negatively charged residue at position 439 and the second CH3 domain comprises a positively charged residue at position 356; and wherein the amino acid residue numbering is based on EU numbering. In some embodiments, i) the positively charged residue is a lysine (K) residue, and the negatively charged residue is an aspartic acid (D) residue; or ii) the positively charged residue is a lysine (K) residue, and the negatively charged residue is a glutamic acid (E) residue; or iii) the positively charged residue is an arginine (R) residue, and the negatively charged residue is an aspartic acid (D) residue; or iv) the positively charged residue is an arginine (R) residue, and the negatively charged residue is a glutamic acid (E) residue. In some embodiments, i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or ii) the first CH3 domain comprises E357K and S364K substitutions and the second CH3 domain comprises L351 D and K370D substitutions, or the first CH3 domain comprises L351D and K370D substitutions and the second CH3 domain comprises E357K and S364K substitutions; or iii) the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions.

In some embodiments according to any one of the heterodimeric proteins described above, i) the first CH3 domain further comprises K392C substitution and the second CH3 domain further comprises D399C substitution, or the first CH3 domain further comprises D399C substitution and the second CH3 domain further comprises K392C substitution; or ii) the first CH3 domain further comprises Y394C substitution and the second CH3 domain further comprises S354C substitution, or the first CH3 domain further comprises S354C substitution and the second CH3 domain further comprises Y394C substitution; or iii) the first CH3 domain further comprises D356C substitution and the second CH3 domain further comprises Y349C substitution, or the first CH3 domain further comprises Y349C substitution and the second CH3 domain further comprises D356C substitution.

In some embodiments according to any one of the heterodimeric proteins described above, the first CH3 domain and the second CH3 domain are human CH3 domains.

In some embodiments according to any one of the heterodimeric proteins described above, the first polypeptide and the second polypeptide each comprises from the N-terminus to the C-terminus at least a portion of an immunoglobulin hinge region, an immunoglobulin heavy chain constant domain 2 (CH2 domain), and the CH3 domain. In some embodiments, the CH2 domains and the CH3 domains form an IgG Fc region. In some embodiments, the Fc region is of the human IgG1 subclass. In some embodiments, the Fc region is of the human IgG4 subclass. In some embodiments, the Fc region further comprises S228P substitution. In some embodiments, the Fc region further comprises N297A substitution.

In some embodiments according to any one of the heterodimeric proteins described above, the first polypeptide and the second polypeptide are antibody heavy chains. In some embodiments, the heterodimeric protein further comprises the heterodimeric protein further comprises one or more antibody light chains. In some embodiments, the heterodimeric protein is a multispecific antibody.

In some embodiments according to any one of the heterodimeric proteins described above, the heterodimeric protein further comprises a third polypeptide and a fourth polypeptide, wherein:

(i) the first polypeptide comprises a structure represented by the formula:

VH1-CH1-hinge-CH2-first CH3-L1-scFv1 (Ia);

(ii) the second polypeptide comprises a structure represented by the formula:

VH2-CH1-hinge-CH2-second CH3-L2-scFv2 (IIa);

(iii) the third polypeptide comprises a structure represented by the formula:

VL1-CL (Ib); and

(iv) the fourth polypeptide comprises a structure represented by the formula

VL2-CL (IIb);

wherein VL1 is a first immunoglobulin light chain variable domain; VH1 is a first immunoglobulin heavy chain variable domain; VL2 is a second immunoglobulin light chain variable domain; VH2 is a second immunoglobulin heavy chain variable domain; scFv1 is a first single-chain variable fragment; scFv2 is a second single-chain variable fragment; CL is an immunoglobulin light chain constant domain; CH1 is an immunoglobulin heavy chain constant domain 1; CH2 is an immunoglobulin heavy chain constant domain 2; hinge is an immunoglobulin hinge region connecting the CH1 and CH2 domains; and L1 and L2 is each independently a bond or a peptide linker; wherein VL1 and VH1 associate to form a first Fv that specifically binds to a first target; wherein VL2 and VH2 associate to form a second Fv that specifically binds to a second target; wherein scFv1 specifically binds to a third target; and wherein scFv2 specifically binds to a fourth target. In some embodiments, scFv1 and scFv2 are identical. In some embodiments, the first Fv and the second Fv are identical. In some embodiments, the first Fv and the second Fv are different. In some embodiments, the first Fv specifically binds PDL1, the second Fv specifically binds CD137, and scFv1 and scFv2 specifically bind CTLA-4. In some embodiments, scFv1 and/or scFv2 comprises from the N-terminus to the C-terminus: VH-L-VL, wherein L is a peptide linker. In some embodiments, scFv1 and/or scFv2 comprises a first cysteine residue at position 44 in the VH and a second cysteine residue at position 100 in the VL, wherein the first cysteine residue and the second cysteine residue form a disulfide bond. In some embodiments, L1 and/or L2 is a peptide linker comprising the amino acid sequence of SEQ ID NO: 80 or SEQ ID NO: 81. In some embodiments, VL1 and VL2 are identical. In some embodiments, VL1 and VL2 are different.

In some embodiments according to any one of the heterodimeric proteins described above, the heterodimeric protein comprises a first polypeptide, a second polypeptide and a third polypeptide, wherein:

(i) the first polypeptide comprises a structure represented by the formula:

VH-CH1-hinge-CH2-first CH3 (IIIa);

(ii) the second polypeptide comprises a structure represented by the formula:

scFv-hinge-CH2-second CH3 (IVa); and

(iii) the third polypeptide comprises a structure represented by the formula:

VL-CL (IIIb);

wherein VL is an immunoglobulin light chain variable domain; VH is an immunoglobulin heavy chain variable domain; scFv is a single-chain variable fragment; CL is an immunoglobulin light chain constant domain; CH1 is an immunoglobulin heavy chain constant domain 1; CH2 is an immunoglobulin heavy chain constant domain 2; and hinge is an immunoglobulin hinge region connecting the CH1 and CH2 domains; wherein VL and VH associate to form an Fv that specifically binds to a first target; and wherein the scFv specifically binds to a second target. In some embodiments, the first target is a tumor antigen, and the second target is CD3. In some embodiments, the first target is HER2. In some embodiments, the first target is a first immune checkpoint molecule, and the second target is a second immune checkpoint molecule. In some embodiments, the first target is PDL1 and the second target is CD137. In some embodiments, the first target is CD137 and the second target is PDL1. In some embodiments, the scFv comprises from the N-terminus to the C-terminus: VH-L-VL, wherein L is a peptide linker. In some embodiments, the scFv comprises a first cysteine residue at position 44 in the VH and a second cysteine residue at position 100 in the VL, wherein the first cysteine residue and the second cysteine residue form a disulfide bond. In some embodiments, the scFv is fused to the hinge in the second polypeptide via a peptide linker comprising the amino acid sequence of SEQ ID NO: 80 or SEQ ID NO: 81.

In some embodiments according to any one of the heterodimeric proteins described above, the heterodimeric protein is an activatable antibody, wherein the heterodimeric protein comprises a first polypeptide, a second polypeptide and a third polypeptide, and wherein:

(i) the first polypeptide comprises a structure represented by the formula:

VH-CH1-hinge-CH2-first CH3 (Va);

(ii) the second polypeptide comprises a structure represented by the formula:

MM1-CM1-scFv-hinge-CH2-second CH3 (VIa); and

(iii) the third polypeptide comprises a structure represented by the formula:

MM2-CM2-VL-CL (IVb);

wherein VL is an immunoglobulin light chain variable domain; VH is an immunoglobulin heavy chain variable domain; scFv is a single-chain variable fragment; CL is an immunoglobulin light chain constant domain; CH1 is an immunoglobulin heavy chain constant domain 1; CH2 is an immunoglobulin heavy chain constant domain 2; hinge is an immunoglobulin hinge region connecting the CH1 and CH2 domains; MM1 is a first masking peptide; MM2 is a second masking peptide; CM1 is a first cleavable peptide; and CM2 is a second cleavable peptide; wherein VL and VH associate to form a first Fv that specifically binds to a first target; wherein the scFv specifically binds to a second target; wherein MM1 inhibits the binding of the first Fv to the first target when CM1 is not cleaved; and wherein MM2 inhibits the binding of the scFv to the second target when CM2 is not cleaved. In some embodiments, the first target is a tumor antigen, and the second target is CD3. In some embodiments, MM1 comprises the amino acid sequence of SEQ ID NO: 35. In some embodiments, the first Fv comprises a VH comprising an CDR-H1 comprising the amino acid sequence of SEQ ID NO: 61, a CDR-H2 comprising the amino acid sequence of SEQ ID NO: 62, and/or a CDR-H3 comprising the amino acid sequence of SEQ ID NO: 63. In some embodiments, the TBM comprises a VL comprising a CDR-L1 comprising the amino acid sequence of SEQ ID NO: 64, a CDR-L2 comprising the amino acid sequence of SEQ ID NO: 65, and/or a CDR-L3 comprising the amino acid sequence of SEQ ID NO: 66. In some embodiments, the first target is HER2. In some embodiments, MM2 comprises the amino acid sequence of SEQ ID NO: 36. In some embodiments, the scFv comprises a VH comprising an CDR-H1 comprising the amino acid sequence of SEQ ID NO: 69, a CDR-H2 comprising the amino acid sequence of SEQ ID NO: 70, and/or a CDR-H3 comprising the amino acid sequence of SEQ ID NO: 71. In some embodiments, the TBM comprises a VL comprising a CDR-L1 comprising the amino acid sequence of SEQ ID NO: 72, a CDR-L2 comprising the amino acid sequence of SEQ ID NO: 73, and/or a CDR-L3 comprising the amino acid sequence of SEQ ID NO: 74.

One aspect of the present application provides an activatable antibody comprising: a first polypeptide comprising, from N-terminus to C-terminus, a masking moiety (MM), a cleavable moiety (CM), and a target binding moiety (TBM), wherein the MM comprises the amino acid sequence of SEQ ID NO: 35; wherein the MM inhibits binding of the activatable antibody to human CD3 when the CM is not cleaved; wherein the CM comprises at least a first cleavage site; and wherein: a) the TBM comprises a VL and the activatable antibody further comprises a second polypeptide comprising a VH; b) the TBM comprises a VH and the activatable antibody further comprises a second polypeptide comprising a VL; c) the TBM comprises from the N-terminus to the C-terminus, a VL and a VH; or d) the TBM comprise from the N-terminus to the C-terminus, a VH and a VL; and wherein the activatable antibody binds to human CD3 via the VH and VL when the CM is cleaved. In some embodiments, the TBM comprises a VH comprising an CDR-H1 comprising the amino acid sequence of SEQ ID NO: 61, a CDR-H2 comprising the amino acid sequence of SEQ ID NO: 62, and/or a CDR-H3 comprising the amino acid sequence of SEQ ID NO: 63. In some embodiments, the TBM comprises a VL comprising a CDR-L1 comprising the amino acid sequence of SEQ ID NO: 64, a CDR-L2 comprising the amino acid sequence of SEQ ID NO: 65, and/or a CDR-L3 comprising the amino acid sequence of SEQ ID NO: 66.

One aspect of the present application provides an activatable antibody comprising: a first polypeptide comprising, from N-terminus to C-terminus, a masking moiety (MM), a cleavable moiety (CM), and a target binding moiety (TBM), wherein the MM comprises the amino acid sequence of SEQ ID NO: 36; wherein the MM inhibits binding of the activatable antibody to human HER2 when the CM is not cleaved; wherein the CM comprises at least a first cleavage site; and wherein a) the TBM comprises a VL and the activatable antibody further comprises a second polypeptide comprising a VH; b) the TBM comprises a VH and the activatable antibody further comprises a second polypeptide comprising a VL; c) the TBM comprises from the N-terminus to the C-terminus, a VL and a VH; or d) the TBM comprise from the N-terminus to the C-terminus, a VH and a VL; and wherein the activatable antibody binds to human HER2 via the VH and VL when the CM is cleaved. In some embodiments, the TBM comprises a VH comprising an CDR-H1 comprising the amino acid sequence of SEQ ID NO: 69, a CDR-H2 comprising the amino acid sequence of SEQ ID NO: 70, and/or a CDR-H3 comprising the amino acid sequence of SEQ ID NO: 71. In some embodiments, the TBM comprises a VL comprising a CDR-L1 comprising the amino acid sequence of SEQ ID NO: 72, a CDR-L2 comprising the amino acid sequence of SEQ ID NO: 73, and/or a CDR-L3 comprising the amino acid sequence of SEQ ID NO: 74.

In some embodiments according to any one of activatable antibodies described above, the activatable antibody comprises a first polypeptide, a second polypeptide and a third polypeptide, wherein:

(i) the first polypeptide comprises a structure represented by the formula:

VH-CH1-hinge-CH2-first CH3 (Va);

(ii) the second polypeptide comprises a structure represented by the formula:

MM1-CM1-scFv-hinge-CH2-second CH3 (VIa); and

(iii) the third polypeptide comprises a structure represented by the formula:

MM2-CM2-VL-CL (IVb);

wherein:
VL is an immunoglobulin light chain variable domain;
VH is an immunoglobulin heavy chain variable domain;
scFv is a single-chain variable fragment;
CL is an immunoglobulin light chain constant domain;
CH1 is an immunoglobulin heavy chain constant domain 1;
   CH2 is an immunoglobulin heavy chain constant domain 2;
   hinge is an immunoglobulin hinge region connecting the CH1 and CH2 domains;
   MM1 is a first masking peptide;
   MM2 is a second masking peptide;
   CM1 is a first cleavable peptide; and
   CM2 is a second cleavable peptide;
      wherein VL and VH associate to form a first Fv that specifically binds to a first target; wherein the scFv specifically binds to a second target; and wherein MM is MM1 or MM2.

In some embodiments according to any one of activatable antibodies described above, the activatable antibody comprises an Fc region comprising a first CH3 domain and a second CH3 domain, wherein the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions.

One aspect of the present application provides one or more nucleic acid(s) encoding the heterodimeric protein according to any one of the heterodimeric proteins described above or the activatable antibody according to any one of the activatable antibodies described above, vector(s) comprising the one or more nucleic acids, and a host cell comprising the one or more nucleic acids or the vector. In some embodiments, there is provided a method for preparing a heterodimeric protein or an activatable antibody, comprising: (a) culturing the host cell according to any one of the host cells described above under conditions that allow expression of the one or more nucleic acid(s) or vector; and (b) recovering the heterodimeric protein or the activatable antibody from the host cell culture.

One aspect of the present application provides a pharmaceutical composition comprising the heterodimeric protein according to any one of the heterodimeric proteins described above or the activatable antibodies according to any one of the activatable antibodies described above, and a pharmaceutically acceptable carrier.

One aspect of the present application provides a method for treating a disease or condition in a subject in need thereof, comprising administering to the subject an effective amount of the pharmaceutical composition according to any one of the pharmaceutical compositions described above. In some embodiments, the disease or condition is cancer. In some embodiments, the cancer is lung cancer. In some embodiments, the cancer is a HER-2 positive cancer. In some embodiments, the cancer is ovarian cancer. In some embodiments, the cancer is prostate cancer or melanoma. In some embodiments, the cancer is an advanced-stage cancer.

Compositions, uses, kits and articles of manufacture comprising any one of the heterodimeric proteins described above are also provided.

### BRIEF DESCRIPTION OF THE DRAWINGS

FIGS. 1-5 provide schematic diagrams of exemplary antibody designs of the present application.

FIG. 1A shows a Fab-Fc/Fc one-armed scaffold schematic. FIG. 1B shows a common light chain scaffold schematic.

FIG. 2 shows a Morrison format bispecific scaffold schematic. Shown at right are PD-L1×CD137 and CD137×PD-L1 bispecific antibodies in the Morrison format.

FIG. 3 shows trispecific scaffold schematics, including, at right, a trispecific antibody to PD-L1, CD137, and CTLA4.

FIG. 4 shows ScFv bispecific scaffold schematics, including, at right, a HER2 and CD3 bispecific antibody schematic.

FIG. 5 shows activatable scaffold schematics, including, at right, a schematic of an activatable antibody to HER2 and CD3. The masking peptide (represented as a ball) can be fused to the antigen-binding domain via a cleavable linker.

FIG. 6 provides 10% SDS-PAGE gels showing the yields of heterodimeric proteins. The three bands correspond to light chain-heavy chain homodimers, light chain-heavy chain-Fc heterodimers, and light chain-heavy chain half-bodies, as indicated.

FIG. 7 provides size-exclusion high-performance liquid chromatography data of heterodimeric proteins. Time is on the x-axis, and relative protein abundance is on the y-axis. The stars indicate peaks corresponding to heterodimeric proteins.

FIG. 8 provides analyzed size-exclusion high-performance liquid chromatography data showing heterodimeric proteins after 1-hour incubations at the temperatures indicated. The x-axis indicates temperature (from left to right, a control, 40° C., 50° C., 60° C., 65′C, or 67.5° C.), and the y-axis indicates the peak area corresponding to the heterodimeric protein post incubation relative to that of control (without incubation).

FIG. 9 provides size-exclusion high-performance liquid chromatography spectra of heterodimeric proteins after storage at 4° C. or 37C for 7, 14, 21, or 28 days, as indicated. Time is on the x-axis, and relative protein abundance is on the y-axis.

FIG. 10 shows the effects of bispecific antibodies in an NFκB activation luciferase reporter assay. The x-axis indicates the log-transformed concentration of antibody in nM, and the y-axis indicates relative luminescence units (“RLU”) of the luciferase reporter.

FIGS. 11A-11C provide an evaluation of the quality of purified anti-PDL1 and CD137 bispecific antibodies of different formats. FIG. 11A shows protein quality evaluated by analytical size-exclusion chromatography after for 3 and 6 freeze-thaw cycles. FIG. 11B shows protein quality evaluated by analytical size-exclusion chromatography after incubation at 40° C. for 28 days. In FIG. 11A and FIG. 11B, time is on the x-axis, and relative protein abundance is on the y-axis, and the format of the bispecific antibody is indicated. FIG. 11C provides an analysis of the thermal stability of purified anti-PDL1 and CD137 bispecific antibodies of different formats. Antibodies were incubated for one hour at the temperature indicated on the x-axis (from left to right, a control, 50 C, 60° C., 65° C., and 70 C), and the y-axis indicates the percentage of main peak area. In FIG. 11C, PDL1×CD137 TYF01 is indicated as squares, CD137×PDL1 TYF01 is shown as circles, PDL1×CD137 TYF02 is shown as upward-pointing triangles, and CD137×PDL1 TYF02 is shown as downward-pointing triangles.

FIG. 12 provides a flow cytometry analysis of binding of anti-PDL1×CD137 bispecific antibodies to PDL1 and CD137. The x-axis of each plot shows antigen display in the APC channel, and the y-axis shows ligand binding in the PE channel.

FIG. 13 provides a flow cytometry analysis of anti-PDL1 and CD137 bispecific antibodies binding and cross-reacting. TYF01 antibodies are shown on the top set of plots, and TYF02 antibodies are shown on the bottom set of plots. The x-axis of each plot shows antigen display in the FITC channel, and the y-axis shows antibody binding in the APC channel. The ability of the bispecific antibodies of different formats to bind PDL1 or CD137 of mouse, monkey, or human origin was tested, as indicated.

FIG. 14 provides the effect of PDL1×CD137 and CD137×PDL1 bispecific antibodies on PDL1 and CD137 reporter gene assays. The top plot shows a PDL1 reporter gene assay, with the log-transformed concentration of antibody on the x-axis in ng/ml, and the relative luminescence units (“RUL”) on the y-axis. In the PDL1 reporter gene assay plot, squares indicate PDL1×CTLA4 bispecific antibody, downward-facing triangles indicate PDL1 monomer, circles indicate PDL1×CD137 bispecific antibody, and upward-facing triangles indicate CD137×PDL1 bispecific antibody. The lower plot shows a CD137-NFκB reporter gene assay, with the concentration of antibody on the x-axis in nM, and the relative luminescence units (“RLU”) on the y-axis. In the CD137-NFκB reporter assay plot, squares indicate PDL1×CD137 bispecific antibody, upward-facing triangles indicate CD137×PDL1 bispecific antibody, diamonds indicate CD137×CTLA4 bispecific antibody, downward-facing triangles indicate CD137 monomer, and circles indicate a negative control.

FIGS. 15A-15C show the effect of anti-PDL1 and/or anti-CD137 antibodies on tumor growth in vivo in a 3LL syngeneic mouse model. In each of FIG. 15A, FIG. 15B, and FIG. 15C the x-axis indicates the number of days after the start of treatment, and the y-axis indicates the tumor volume in mm. Mono-IgG, bispecific or trispecific antibodies were tested alone or in combination at the concentrations indicated.

FIG. 16 provides a characterization of bispecific antibodies through SDS-PAGE electrophoresis. The left gel is a 12% SDS-PAGE gel under reducing conditions, and the right gel is a 4-15% SDS-PAGE gel under non-reducing conditions. The MW lane shows molecular weight markers, which are labeled in kilodaltons to the left of each gel. In both gels, lane 1 shows antibody TY24051, lane 2 shows antibody TY24052, and lane 3 shows antibody TY24053.

FIG. 17 provides size-exclusion high-performance liquid chromatography analyses of bispecific antibodies. The upper plot shows antibody TY24051, the middle plot shows antibody TY24105, and the lower plot shows antibody TY24106. In each plot, time is on the x-axis, and relative protein abundance is on the y-axis. Peaks corresponding to heterodimeric proteins and aggregates are indicated.

FIGS. 18A-18B provide enzyme-linked immunosorbent assay (ELISA) analyses of antibodies TY24051 and TY24052. FIG. 18A shows binding of HER2 by TY24051 (squares), TY24052 (triangles pointing up), and TY24052 after activation (triangles pointing down). FIG. 18B shows binding of CD3 by TY24051 (squares), TY24052 (triangles pointing up), and TY24052 after activation (triangles pointing down). In both FIG. 18A and FIG. 18B, the concentration of antibody is on the x-axis in M, and the absorbance at 450 nm is on the y-axis.

FIG. 19 shows an assay of T-cell mediated cytotoxic killing upon treatment with bispecific antibodies. The concentration of antibody (ng/ml) is shown on the x-axis, and the percentage of cell lysis is shown on the y-axis. Target cells were incubated with T cells for 24 hours with TY24051 (circles), TY24052 (squares), an isotype control (triangles pointing up), or without an antibody (triangles pointing down).

### DETAILED DESCRIPTION OF THE INVENTION

The present application provides heterodimeric proteins comprising CH3 domains having engineered disulfide bond(s) and/or salt bridge(s), including multispecific antibodies such as bispecific antibodies comprising an Fc region having the engineered disulfide bond(s) and/or salt bridge(s). In some embodiments, the heterodimeric protein comprises an engineered disulfide bond between C390 in a first CH3 domain and C400 in a second CH3 domain, between C392 in a first CH3 domain and C397 in a second CH3 domain, or between C392 in a first CH3 domain and C400 in a second CH3 domain. In some embodiments, the heterodimeric protein comprises a rearranged salt-bridge network as compared to wildtype CH3 domains, e.g., among positions 357 and 411 in a first CH3 domain and positions 351 and 370 in a second CH3 domain (e.g., E357K:T411K-L351′D:K370′D), or among positions 357 and 364 in a first CH3 domain and positions 351 and 370 in a second CH3 domain (e.g., E357K:S364K-L351′D:K370′D). In some embodiments, the heterodimeric protein comprises an inversed salt bridge as compared to wildtype CH3 domains between position 356 in a first CH3 domain and position 439 in a second CH3 domain (e.g., D356-K439′). The heterodimeric proteins described herein provide a platform for preparing various formats of multispecific proteins and antibodies with high yield, superior stability (e.g., resistance to aggregation and precipitation at high temperature or due to freeze-thaw cycles), and potent activity.

### I. DEFINITIONS

Terms are used herein as generally used in the art, unless otherwise defined as follows.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “antibody” includes, but is not limited to, fragments that are capable of binding antigen, such as Fv, Fab, Fab′, and (Fab′)₂. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc.

The term “antigen-binding fragment” refers to one or more portions of an antibody that retain the ability to bind to the antigen of the antibody. Examples of “antigen-binding fragment” of an antibody include, but are not limited to, (i) a Fab fragment, a monovalent fragment consisting of the VL, VH. CL and CH1 domains; (ii) a F(ab′)₂ fragment, a bivalent fragment comprising two Fab fragments linked by a disulfide bridge at the hinge region; (iii) a Fv fragment consisting of the VL and VH domains of a single arm of an antibody, (v) a single chain Fv fragment comprising the VH and VL domains of an antibody, and the VH and VL domains are fused to each other; and (vi) a single chain Fab fragment comprising a single polypeptide comprising the VL, VH, CL and CH1 domains.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hypervariable region” or “HVR,” as used herein, refers to each of the regions of an antibody variable domain, which are hypervariable in sequence. HVRs may form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), CDRs being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008)). Unless otherwise indicated, HVR residues and other residues in the variable domain (e.g., FR residues) are numbered herein according to Kabat et al., supra.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four framework regions (FRs) and three hypervariable regions (HVRs), arranged from amino-terminus to carboxy-terminus in the following order: FR1, HVR1, FR2, HVR2, FR3, HVR3, FR4. (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “EU numbering” or “amino acid position numbering based on EU numbering,” and variations thereof, refers to the numbering system used for heavy chain constant domains of the compilation of antibodies in Edelman, G. M. et al., Proc. Natl. Acad. USA, 63, 78-85 (1969). The EU numbering of residues may be determined for a given antibody by alignment at regions of homology of the sequence of the antibody with a “standard” EU numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

With respect to a heterodimeric protein having two CH3 domains, a given amino acid position of a first CH3 domain is referred to as X, and the corresponding amino acid position of the second CH3 domain is referred to as X′. For example, N390C-S400′C refers to a heterodimeric protein having a first CH3 domain having a N390C mutation and a second CH3 domain having a S400C mutation. All mutations or substitutions in the heterodimeric proteins described herein are referred herein with respect to a wildtype, naturally occurring CH3 domain.

Unless indicated otherwise, all formula of polypeptide chains described herein list the components of the polypeptide in the order from the N-terminus to the C-terminus. For example, the formula VH1-CH I-hinge-CH2-first CH3-L1-scFv1 indicates that the polypeptide comprises, from the N-terminus to the C-terminus, the following structural components: VH1, CH1, hinge, CH2, first CH3, L1 and scFv1.

The term “heavy chain constant region” as used herein refers to a region comprising at least three heavy chain constant domains, CH1, CH2, and CH3, and a hinge region between CH1 and CH2. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a 73 constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “CH2 domain” of a human IgG Fc region usually extends from about residues 231 to about 340 of the IgG according to the EU numbering system. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The term “CH3 domain” comprises the stretch of residues C-terminal to a CH2 domain in an Fc region (i.e., from about amino acid residue 341 to about amino acid residue 447 of an IgG according to the EU numbering system).

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain constant region” as used herein refers to a region comprising a light chain constant domain, CL. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein. In the context of a multispecific antibody (e.g., a bispecific or trispecific antibody), affinity of the antibody with each binding specificity (i.e. target) can be measured.

The term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In some embodiments, the extent of binding of an antibody to an unrelated target is less than about 10% of the binding of the antibody to the target as measured, e.g., by a radioimmunoassay (RIA). In some embodiments, an antibody that specifically binds to a target has a dissociation constant (K_{d}) of ≤1 μM, ≤100 nM, ≤10 nM, ≤1 nM, or ≤0.1 nM. In some embodiments, an antibody specifically binds to an epitope on a protein that is conserved among the protein from different species. In some embodiments, specific binding can include, but does not require exclusive binding.

The term “multispecific” as used in conjunction with an antibody refers to an antibody having polyepitopic specificity (i.e., is capable of specifically binding to two, three, or more, different epitopes on one biological molecule or is capable of specifically binding to epitopes on two, three, or more, different biological molecules).

An “affinity matured” antibody refers to an antibody with one or more alterations in one or more hypervariable regions (HVRs) compared to a parent antibody, which does not possess such alterations, such alterations resulting in an improvement in the affinity of the antibody for antigen. In some examples, an affinity-matured antibody refers to an antibody with one or more alterations in one or more complementarity determining regions (CDRs) compared to a parent antibody, which does not possess such alterations, such alterations resulting in an improvement in the affinity of the antibody for antigen.

A “chimeric antibody” as used herein refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, a (Fab′)₂, etc.

An “HVR-grafted antibody” as used herein refers to a humanized antibody in which one or more hypervariable regions (HVRs) of a first (non-human) species have been grafted onto the framework regions (FRs) of a second (human) species. In some examples, a “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequence.

The terms “nucleic acid molecule”, “nucleic acid” and “polynucleotide” may be used interchangeably, and refer to a polymer of nucleotides. Such polymers of nucleotides may contain natural and/or unnatural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “peptide” are used interchangeably to refer to a polymer of amino acid residues, and are not limited to a minimum length. Such polymers of amino acid residues may contain natural or unnatural amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” includes modifications, such as deletions, additions, and substitutions (generally conservative in nature), to the native sequence, as long as the polypeptide maintains the desired activity. These modifications may be deliberate, as through site-directed mutagenesis, or may be accidental, such as through mutations of hosts, which produce the proteins or errors due to PCR amplification.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein. “percent (%) amino acid sequence identity” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table A. Amino acid substitutions may be introduced into an antibody of interest and the products screened for a desired activity, e.g., retained/improved antigen binding, decreased immunogenicity, or improved ADCC or CDC.

TABLE A
Exemplary Amino Acid Substitutions.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:

(1) hydrophobic: Norleucine, Met, Ala. Val, Leu, Ile:

(2) neutral hydrophilic: Cys, Ser, Thr, Asn, Gln;

(3) acidic: Asp, Glu;

(4) basic: His, Lys, Arg:

(5) residues that influence chain orientation: Gly, Pro:

(6) aromatic: Trp, Tyr, Phe.

Non-conservative substitutions will entail exchanging a member of one of these classes for another class.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays. e.g., D-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells: fungal cells, such as yeast; plant cells; and insect cells. Non-limiting exemplary mammalian cells include, but are not limited to, NSO cells, PER.C6® cells (Crucell), and 293 and CHO cells, and their derivatives, such as 293-6E and DG44 cells, respectively. The term “cell” includes the primary subject cell and its progeny.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual” or “subject” are used interchangeably herein to refer to a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer means to defer, hinder, slow, retard, stabilize, and/or postpone development of the disease. This delay can be of varying lengths of time, depending on the history of the disease and/or individual being treated. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of an agent or a combination of agents, sufficient to treat a specified disorder, condition or disease such as to ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other undesired cell proliferation. In some embodiments, an effective amount is an amount sufficient to delay disease development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size: (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a”, “an”, and “the” include plural referents unless the context clearly dictates otherwise.

The term “and/or” as used herein a phrase such as “A and/or B” is intended to include both A and B: A or B: A (alone); and B (alone). Likewise, the term “and/or” as used herein a phrase such as “A, B, and/or C” is intended to encompass each of the following embodiments: A, B, and C; A, B. or C; A or C; A or B; B or C; A and C; A and B: B and C; A (alone); B (alone); and C (alone).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the heterodimeric proteins are specifically embraced by the present invention and are disclosed herein just as if each and every combination was individually and explicitly disclosed. In addition, all subcombinations of the heterodimeric proteins listed in the embodiments describing such variables are also specifically embraced by the present invention and are disclosed herein just as if each and every such sub-combination of the heterodimeric proteins was individually and explicitly disclosed herein.

### II. HETERODIMERIC PROTEINS

The present application provides heterodimeric proteins comprising CH3 domains having any one or combination of engineered residues, which promote heterodimer formation as described in the subsection “CH3 domain mutations.” Heteromultimers comprising multiple heterodimers formed by a first polypeptide comprising a first engineered CH3 domain and a second polypeptide comprising a second engineered CH3 domain are also contemplated herein.

In some embodiments, there is provided a heterodimeric protein (e.g., multispecific antibody) comprising a first polypeptide comprising a first CH3 domain and a second polypeptide comprising a second CH3 domain, wherein: i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and wherein the amino acid residue numbering is based on EU numbering.

In some embodiments, there is provided a heterodimeric protein (e.g., multispecific antibody) comprising a first polypeptide comprising a first CH3 domain and a second polypeptide comprising a second CH3 domain, wherein: i) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or ii) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or iii) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of i) and ii), or a combination or i) and iii), wherein the amino acid residue numbering is based on EU numbering. In some embodiments, the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356, and wherein the amino acid residue numbering is based on EU numbering.

In some embodiments, there is provided a heterodimeric protein (e.g., multispecific antibody) comprising a first polypeptide comprising a first CH3 domain and a second polypeptide comprising a second CH3 domain, wherein: i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and wherein: a) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or b) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or c) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of a) and b), or a combination of a) and c); wherein the amino acid residue numbering is based on EU numbering. In some embodiments, the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356, and wherein the amino acid residue numbering is based on EU numbering.

The CH3 domains may be derived from any naturally occurring immunoglobulin molecules. In some embodiments, the CH3 domains are derived from an IgG1 molecule, an IgG2 molecule, an IgG3 molecule, or an IgG4 molecule. In some embodiments, the CH3 domains are human CH3 domains. In some embodiments, the CH3 domains are derived from human IgG1 molecules.

In some embodiments, there is provided a heterodimeric protein (e.g., multispecific antibody) comprising a first polypeptide comprising a first CH3 domain and a second polypeptide comprising a second CH3 domain, wherein: i) the first CH3 domain comprises N390C substitution and the second CH3 domain comprises S400C substitution, or the first CH3 domain comprises S400C substitution and the second CH3 domain comprises N390C substitution; or ii) the first CH3 domain comprises K392C substitution and the second CH3 domain comprises V397C substitution, or the first CH3 domain comprises V397C substitution and the second CH3 domain comprises K392C substitution; or iii) the first CH3 domain comprises K392C substitution and the second CH3 domain comprises S400C substitution, or the first CH3 domain comprises S400C substitution and the second CH3 domain comprises K392C substitution.

In some embodiments, there is provided a heterodimeric protein (e.g., multispecific antibody) comprising a first polypeptide comprising a first CH3 domain and a second polypeptide comprising a second CH3 domain, wherein: i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or ii) the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions; or iii) the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions.

In some embodiments, there is provided a heterodimeric protein (e.g., multispecific antibody) comprising a first polypeptide comprising a first CH3 domain and a second polypeptide comprising a second CH3 domain, wherein the first CH3 domain comprises E357K, S364K and N390C substitutions and the second CH3 domain comprises L351D, K370D, and S400C substitutions, or the first CH3 domain comprises L351D, K370D, and S400C substitutions and the second CH3 domain comprises E357K, S364K and N390C substitutions.

In some embodiments, there is provided a heterodimeric protein (e.g., multispecific antibody) comprising a first polypeptide comprising a first CH3 domain and a second polypeptide comprising a second CH3 domain, wherein the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions.

In some embodiments, there is provided a heterodimeric protein (e.g., multispecific antibody) comprising a first polypeptide comprising a first CH3 domain and a second polypeptide comprising a second CH3 domain, wherein the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions.

In some embodiments, there is provided a heterodimeric protein (e.g., multispecific antibody) comprising a first polypeptide comprising a first CH3 domain and a second polypeptide comprising a second CH3 domain, wherein the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions.

In some embodiments, the heterodimeric protein comprises an IgG Fc region that comprises the engineered CH3 domains. The Fc region may be derived from any suitable Fc subclasses, including, but not limited to, IgG1, IgG2, IgG3, and IgG4 subclasses.

Tables 1 A-1B in the Examples section lists exemplary polypeptide sequences (SEQ ID NOs: 1-28) of CH3 domains (or Fc regions) comprising the engineered disulfide bond(s) and/or salt bridge(s) described herein. Other polypeptide sequences of engineered CH3 domains or polypeptides of the Fc regions include SEQ ID NOs: 138-365. Also provided are polypeptides comprising an amino acid sequence selected from the group consisting of SEQ ID NOs: 1-28 and 138-365.

In some embodiments, there is provided a heterodimeric protein (e.g., multispecific antibody) comprising a first polypeptide comprising the amino acid sequence of SEQ ID NO: 1, and a second polypeptide comprising the amino acid sequence of SEQ ID NO: 2. In some embodiments, there is provided a heterodimeric protein (e.g., multispecific antibody) comprising a first polypeptide comprising the amino acid sequence of SEQ ID NO: 3, and a second polypeptide comprising the amino acid sequence of SEQ ID NO: 4. In some embodiments, there is provided a heterodimeric protein (e.g., multispecific antibody) comprising a first polypeptide comprising the amino acid sequence of SEQ ID NO: 5, and a second polypeptide comprising the amino acid sequence of SEQ ID NO: 6. In some embodiments, there is provided a heterodimeric protein (e.g., multispecific antibody) comprising a first polypeptide comprising the amino acid sequence of SEQ ID NO: 7, and a second polypeptide comprising the amino acid sequence of SEQ ID NO: 8. In some embodiments, there is provided a heterodimeric protein (e.g., multispecific antibody) comprising a first polypeptide comprising the amino acid sequence of SEQ ID NO: 9, and a second polypeptide comprising the amino acid sequence of SEQ ID NO: 10. In some embodiments, there is provided a heterodimeric protein (e.g., multispecific antibody) comprising a first polypeptide comprising the amino acid sequence of SEQ ID NO: 11, and a second polypeptide comprising the amino acid sequence of SEQ ID NO: 12. In some embodiments, there is provided a heterodimeric protein (e.g., multispecific antibody) comprising a first polypeptide comprising the amino acid sequence of SEQ ID NO: 13, and a second polypeptide comprising the amino acid sequence of SEQ ID NO: 14. In some embodiments, there is provided a heterodimeric protein (e.g., multispecific antibody) comprising a first polypeptide comprising the amino acid sequence of SEQ ID NO: 15, and a second polypeptide comprising the amino acid sequence of SEQ ID NO: 16. In some embodiments, there is provided a heterodimeric protein (e.g., multispecific antibody) comprising a first polypeptide comprising the amino acid sequence of SEQ ID NO: 17, and a second polypeptide comprising the amino acid sequence of SEQ ID NO: 18. In some embodiments, there is provided a heterodimeric protein (e.g., multispecific antibody) comprising a first polypeptide comprising the amino acid sequence of SEQ ID NO: 19, and a second polypeptide comprising the amino acid sequence of SEQ ID NO: 20. In some embodiments, there is provided a heterodimeric protein (e.g., multispecific antibody) comprising a first polypeptide comprising the amino acid sequence of SEQ ID NO: 21, and a second polypeptide comprising the amino acid sequence of SEQ ID NO: 22. In some embodiments, there is provided a heterodimeric protein (e.g., multispecific antibody) comprising a first polypeptide comprising the amino acid sequence of SEQ ID NO: 23, and a second polypeptide comprising the amino acid sequence of SEQ ID NO: 24.

### CH3 Domain Mutations

The heterodimeric proteins described herein may have one or more engineered disulfide bonds, one or more engineered (e.g., rearranged or inversed) salt bridges, or a combination thereof. Unless stated otherwise, all amino acid residue numbering herein is based on EU numbering, and the amino acid substitutions are relative to the wildtype (or naturally occurring) sequence at the corresponding amino acid positions in a wild type (or naturally occurring) CH3 domain sequence. It is appreciated that the mutations or substitutions described herein are applicable to all IgG subclasses and allotypes. IgG allotypes have been described, for example, in Jefferis R and Lefranc M, mAbs 1:4, 1-7 (2009), which is incorporated herein by reference in its entirety. In some embodiments, the amino acid mutations or substitutions described herein are relative to a wildtype CH3 domain sequence of an IgG1, such as IgG1 allotype G1m, 1(a), 2(x), 3(f) or 17(z). In some embodiments, the amino acid mutations or substitutions described herein are relative to a wildtype CH3 domain sequence of an IgG4. For example, a D356K substitution relative to a wildtype CH3 domain of one human IgG1 allotype (Uniprot ID P01857; SEQ ID NO: 29) is equivalent to an E356K substitution relative to a wildtype CH3 domain of a second human IgG1 allotype (SEQ ID NO: 30), or a wildtype CH3 domain of a human IgG4 (SEQ ID NO: 31). Exemplary CH3 domain mutations are shown in Tables 1A-1B. In some embodiments, the amino acid mutations or substitutions described herein are relative to a wildtype Fc region sequence, e.g., an IgG1 Fc region (SEQ ID NO: 32 or 33) or an IgG4 Fc region (SEQ ID NO: 34).

### Novel Cysteine Mutations

In some embodiments, the heterodimeric proteins described herein comprise a first polypeptide comprising a first CH3 domain and a second polypeptide comprising a second CH3 domain, wherein the first CH3 domain comprises a first engineered cysteine residue and the second CH3 domain comprises a second engineered cysteine residue, wherein the first engineered cysteine residue and the second cysteine residue form a disulfide bond.

In some embodiments, the first CH3 domain comprises a C at position 390 and the second CH3 domain comprises a C at position 400, or the first CH3 domain comprises a C at position 400 and the second CH3 domain comprises a C at position 390. In some embodiments, the first CH3 domain comprises N390C substitution and the second CH3 domain comprises S400C substitution, or the first CH3 domain comprises S400C substitution and the second CH3 domain comprises N390C substitution.

In some embodiments, the first CH3 domain comprises a C at position 392 and the second CH3 domain comprises a C at position 397, or the first CH3 domain comprises a C at position 397 and the second CH3 domain comprises a C at position 392. In some embodiments, the first CH3 domain comprises K392C substitution and the second CH3 domain comprises V397C substitution, or the first CH3 domain comprises V397C substitution and the second CH3 domain comprises K392C substitution.

In some embodiments, the first CH3 domain comprises a C at position 392 and the second CH3 domain comprises a C at position 400, or the first CH3 domain comprises a C at position 400 and the second CH3 domain comprises a C at position 392. In some embodiments, the first CH3 domain comprises K392C substitution and the second CH3 domain comprises S400C substitution, or the first CH3 domain comprises S400C substitution and the second CH3 domain comprises K392C substitution.

### Novel Salt Bridge Mutations

In some embodiments, the heterodimeric proteins described herein comprise a first polypeptide comprising a first CH3 domain and a second polypeptide comprising a second CH3 domain, wherein the first CH3 domain comprises an engineered positively charged residue and the second CH3 domain comprises an engineered negatively charged residue, wherein the engineered positively charged residue and the engineered negatively charged residue form a salt bridge. The engineered salt bridge may introduce new salt bridges between the CH3 domains, rearrange a salt-bridge network among two or more amino acid residues, or reverse the charges on the amino acid residues forming the salt bridge (i.e., “inverse” a salt bridge) with respect to wildtype CH3 domains. In some embodiments, the engineered positively charged residue substitutes a negatively charged residue in a wildtype CH3 domain with a positively charged residue. In some embodiments, the engineered negatively charged residue substitutes a positively charged residue in a wildtype CH3 domain with a negatively charged residue. The rearranged and inversed salt bridges may result in changes in the isoelectric points (PI) of the heterodimer and the homodimer comprising the engineered CH3 domains, thereby allowing better separation of the heterodimer from the homodimer in a purification process.

In some embodiments, the first CH3 domain comprises a positively charged residue at position 357 and the second CH3 domain comprises a negatively charged residue at position 351, or the first CH3 domain comprises a negatively charged residue at position 351 and the second CH3 domain comprises a positively charged residue at position 357. In some embodiments, first CH3 domain comprises a K at position 357 and the second CH3 domain comprises a D at position 351, or the first CH3 domain comprises a D at position 351 and the second CH3 domain comprises a K at position 357. In some embodiments, first CH3 domain comprises a K at position 357 and the second CH3 domain comprises an E at position 351, or the first CH3 domain comprises an E at position 351 and the second CH3 domain comprises a K at position 357. In some embodiments, first CH3 domain comprises an R at position 357 and the second CH3 domain comprises a D at position 351, or the first CH3 domain comprises a D at position 351 and the second CH3 domain comprises an R at position 357. In some embodiments, first CH3 domain comprises an R at position 357 and the second CH3 domain comprises an E at position 351, or the first CH3 domain comprises an E at position 351 and the second CH3 domain comprises an R at position 357. In some embodiments, the first CH3 domain comprises E357K substitution and the second CH3 domain comprises L351D substitution, or the first CH3 domain comprises L351D substitution and the second CH3 domain comprises E357K substitution.

In some embodiments, the first CH3 domain comprises a positively charged residue at position 411 and the second CH3 domain comprises a negatively charged residue at position 370, or the first CH3 domain comprises a negatively charged residue at position 370 and the second CH3 domain comprises a positively charged residue at position 411. In some embodiments, first CH3 domain comprises a K at position 411 and the second CH3 domain comprises a D at position 370, or the first CH3 domain comprises a D at position 370 and the second CH3 domain comprises a K at position 411. In some embodiments, first CH3 domain comprises a K at position 411 and the second CH3 domain comprises an E at position 370, or the first CH3 domain comprises an E at position 370 and the second CH3 domain comprises a K at position 411. In some embodiments, first CH3 domain comprises an R at position 411 and the second CH3 domain comprises a D at position 370, or the first CH3 domain comprises a D at position 370 and the second CH3 domain comprises an R at position 411. In some embodiments, first CH3 domain comprises an R at position 411 and the second CH3 domain comprises an E at position 370, or the first CH3 domain comprises an E at position 370 and the second CH3 domain comprises an R at position 411. In some embodiments, the first CH3 domain comprises T411K substitution and the second CH3 domain comprises K370D substitution, or the first CH3 domain comprises K370D substitution and the second CH3 domain comprises T411K substitution.

In some embodiments, the first CH3 domain comprises a positively charged residue at position 364 and the second CH3 domain comprises a negatively charged residue at position 370, or the first CH3 domain comprises a negatively charged residue at position 370 and the second CH3 domain comprises a positively charged residue at position 364. In some embodiments, first CH3 domain comprises a K at position 364 and the second CH3 domain comprises a D at position 370, or the first CH3 domain comprises a D at position 370 and the second CH3 domain comprises a K at position 364. In some embodiments, first CH3 domain comprises a K at position 364 and the second CH3 domain comprises an E at position 370, or the first CH3 domain comprises an E at position 370 and the second CH3 domain comprises a K at position 364. In some embodiments, first CH3 domain comprises an R at position 364 and the second CH3 domain comprises a D at position 370, or the first CH3 domain comprises a D at position 370 and the second CH3 domain comprises an R at position 364. In some embodiments, first CH3 domain comprises an R at position 364 and the second CH3 domain comprises an E at position 370, or the first CH3 domain comprises an E at position 370 and the second CH3 domain comprises an R at position 364. In some embodiments, the first CH3 domain comprises S364K substitution and the second CH3 domain comprises K370D substitution, or the first CH3 domain comprises K370D substitution and the second CH3 domain comprises S364K substitution.

In some embodiments, the first CH3 domain comprises a positively charged residue at position 356 and the second CH3 domain comprises a negatively charged residue at position 439, or the first CH3 domain comprises a negatively charged residue at position 439 and the second CH3 domain comprises a positively charged residue at position 356. In some embodiments, first CH3 domain comprises a K at position 356 and the second CH3 domain comprises a D at position 439, or the first CH3 domain comprises a D at position 439 and the second CH3 domain comprises a K at position 356. In some embodiments, first CH3 domain comprises a K at position 356 and the second CH3 domain comprises an E at position 439, or the first CH3 domain comprises an E at position 439 and the second CH3 domain comprises a K at position 356. In some embodiments, first CH3 domain comprises an R at position 356 and the second CH3 domain comprises a D at position 439, or the first CH3 domain comprises a D at position 439 and the second CH3 domain comprises an R at position 356. In some embodiments, first CH3 domain comprises an R at position 356 and the second CH3 domain comprises an E at position 439, or the first CH3 domain comprises an E at position 439 and the second CH3 domain comprises an R at position 356. In some embodiments, the first CH3 domain comprises D356K substitution and the second CH3 domain comprises K439D substitution, or the first CH3 domain comprises K439D substitution and the second CH3 domain comprises D356K substitution.

Any of the engineered salt bridges described herein may be combined with each other. In some embodiments, the first CH3 domain comprises a positively charged residue at position 357 and a positively charged residue at position 411 and the second CH3 domain comprises a negatively charged residue at position 351 and a negatively charged residue at position 370, or the first CH3 domain comprises a negatively charged residue at position 351 and a negatively charged residue at position 370 and the second CH3 domain comprises a positively charged residue at position 357 and a positively charged residue at position 411. In some embodiments, the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions.

In some embodiments, the first CH3 domain comprises a positively charged residue at position 357 and a positively charged residue at position 364 and the second CH3 domain comprises a negatively charged residue at position 351 and a negatively charged residue at position 370, or the first CH3 domain comprises a negatively charged residue at position 351 and a negatively charged residue at position 370 and the second CH3 domain comprises a positively charged residue at position 357 and a positively charged residue at position 364. In some embodiments, the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions.

In some embodiments, the first CH3 domain comprises a positively charged residue at position 356, a positively charged residue at position 357, and a positively charged residue at position 364 and the second CH3 domain comprises a negatively charged residue at position 351, a negatively charged residue at position 370, and a negatively charged residue at position 439, or the first CH3 domain comprises a negatively charged residue at position 351, a negatively charged residue at position 370, and a negatively charged residue at position 439 and the second CH3 domain comprises a positively charged residue at position 356, a positively charged residue at position 357, and a positively charged residue at position 364. In some embodiments, the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions.

### Other Mutations

The CH3 domains or the Fc regions described herein may further comprise engineered disulfide bonds and/or salt bridges listed in Table B below.

TABLE B
Exemplary Fc mutations.

| Mutation(s) in first polypeptide chain | Mutation(s) in second polypeptide chain |
|---|---|
| F405L | K409R |
| S364H, F405A | Y349T, T394F |
| S364K, E357Q | L368D, K370S |
| T366W | T366S, L368A, Y407V |
| S354C, T366W | Y349C, T366S, L368A, Y407V |
| T350, L351, F405, Y407 | T350, T366, K392, T394 |
| T350 is T350V, T350I, T350L or T350M | T350 is T350V, T350I, T350L or T350M |
| L351 is L351Y | T366 is T366L, T366I, T366V or T366M |
| F450 is F450A, F450V, F450T or F450S | K392 is K392F, K392L or K392M |
| Y407 is Y407V, Y407A or Y407I | T394 is T394W |
| D399K, E356K | K409D, K392D |
| D221E, P228E, L368E | D221R, P228R, K409R |
| C223E, E225E, P228E, L368E | C223R, E225R, P228R, K409R |
| H435R | None |
| K196Q, S228P, F296Y, E356K, R409K, H435R, L445P | K196Q, S228P, F296Y, R409K, K439E, L445P |
| K409W | D399V/F405T |
| K360E | Q347R |
| Y349C/K360E/K409W | Q347R/S354C/D399V/F405T |
| K360E/K409W | Q347R/D399V/F405T |
| Y349S/K409W | E357W/D399V/F405T |
| Y349S/S354C/K409W | Y349C/E357W/D399V/F405T |
| T366K | L351D |
| Y349E or D and L368E | L351D |
| Y349C/T366W | D356C/T366S/L368A/Y407V/F405K |
| Y349C/T366W/F405K | D356C/T366S/L368A/Y407V |
| Y349C/T366W/K409E | D356C/T366S/L368A/Y407V/F405K |
| Y349C/T366W/K409A | D356C/T366S/L368A/Y407V/F405K |
| S364K | L368D |
| S364K | K370S |
| F405K | K409F |
| F405R | K409F |
| S364K/K409F | L368D/F405R |
| S364K/K409F | K370S/F405R |
| S364K/K409W | K370S/F405R |
| K370E or E356K and K409R | E357K and K409R or K370E |
| 354/364/407 | 347/349/350/351/366/368/370/407 |
| 349 | 351/354/357/360/364/366/368/407 |
| P395K/P396K/V397K | T394D/P395D/P396D |
| F405E/Y407E/K409E | F405K/Y407K |
| M428S/N434S/Y436H | H435R |

In some embodiments, the first CH3 domain further comprises a C at position 392 and the second CH3 domain comprises a C at position 399, or the first CH3 domain comprises a C at position 399 and the second CH3 domain comprises a C at position 392. In some embodiments, the first CH3 domain further comprises K392C substitution and the second CH3 domain further comprises D399C substitution, or the first CH3 domain further comprises D399C substitution and the second CH3 domain further comprises K392C substitution.

In some embodiments, the first CH3 domain further comprises a C at position 394 and the second CH3 domain comprises a C at position 354, or the first CH3 domain comprises a C at position 354 and the second CH3 domain comprises a C at position 394. In some embodiments, the first CH3 domain further comprises Y394C substitution and the second CH3 domain further comprises S354C substitution, or the first CH3 domain further comprises S354C substitution and the second CH3 domain further comprises Y394C substitution.

In some embodiments, the first CH3 domain further comprises a C at position 356 and the second CH3 domain comprises a C at position 349, or the first CH3 domain comprises a C at position 349 and the second CH3 domain comprises a C at position 356. In some embodiments, the first CH3 domain further comprises D356C substitution and the second CH3 domain further comprises Y349C substitution, or the first CH3 domain further comprises Y349C substitution and the second CH3 domain further comprises D356C substitution.

In some embodiments, the first CH3 domain further comprises K392D and K409D substitutions and the second CH3 domain further comprises D356K and D399K substitutions, or the first CH3 domain further comprises D356K and D399K substitutions and the second CH3 domain further comprises K392D and K409D substitutions.

In some embodiments, the first CH3 domain further comprises L368D and K370S substitutions and the second CH3 domain further comprises E357Q and S364K substitutions, or the first CH3 domain further comprises E357Q and S364K substitutions and the second CH3 domain further comprises L368D and K370S substitutions.

In some embodiments, the first CH3 domain further comprises L351K and T366K substitutions and the second CH3 domain further comprises L351D and L368E substitutions, or the first CH3 domain further comprises L351D and L368E substitutions and the second CH3 domain further comprises L351K and T366K substitutions.

In some embodiments, the first CH3 domain further comprises P395K, P396K and V397K substitutions and the second CH3 domain comprises T394D, P395D and P396D substitutions, or the first CH3 domain further comprises T394D, P395D and P396D substitutions and the second CH3 domain further comprises P395K, P396K and V397K substitutions.

In some embodiments, the first CH3 domain further comprises F405E, Y407E and K409E substitutions and the second CH3 domain comprises F405K and Y407K substitutions, or the first CH3 domain further comprises F405K and Y407K substitutions and the second CH3 domain further comprises F405E, Y407E and K409E substitutions.

The heterodimeric proteins comprising engineered CH3 domains disulfide bonds and/or salt bridges described herein may further comprise one or more knob-into-hole residues. “Knob-into-hole” or “KIH” refers to an approach known in the art for making bispecific antibodies also known as the “protuberance-into-cavity” approach (see, e.g., U.S. Pat. No. 5,731,168). In this approach, two immunoglobulin polypeptides (e.g., heavy chain polypeptides) each comprise an interface. An interface of one immunoglobulin polypeptide interacts with a corresponding interface on the other immunoglobulin polypeptide, thereby allowing the two immunoglobulin polypeptides to associate. These interfaces may be engineered such that a “knob” or “protuberance” (these terms may be used interchangeably herein) located in the interface of one immunoglobulin polypeptide corresponds with a “hole” or “cavity” (these terms may be used interchangeably herein) located in the interface of the other immunoglobulin polypeptide. In some embodiments, the hole is of identical or similar size to the knob and suitably positioned such that when the two interfaces interact, the knob of one interface is positionable in the corresponding hole of the other interface. Without wishing to be bound to theory, this is thought to stabilize the heteromultimer and favor formation of the heteromultimer over other species, for example homomultimers. In some embodiments, the KIH approach is used in combination with the engineered disulfide bonds and/or salt bridges described herein to promote the heteromultimerization of two different immunoglobulin polypeptides, creating a bispecific antibody comprising two immunoglobulin polypeptides with binding specificities for different epitopes. In some embodiments, the CH3 domains of the heterodimeric protein described herein do not comprise KIH residues.

In some embodiments, the first CH3 domain further comprises T336S, L368A and Y407V substitutions and the second CH3 domain further comprises T366W substitution, or the first CH3 domain further comprises T366W substitution and the second CH3 domain further comprises T336S, L368A and Y407V substitutions.

In some embodiments, the first CH3 domain comprises L368V and Y407V substitutions and the second CH3 domain comprises T366W substitution, or the first CH3 domain comprises T366W substitution and the second CH3 domain comprises L368V and Y407V substitutions.

### III. MULTISPECIFIC ANTIBODIES

In some embodiments, the heterodimeric protein described herein is a multispecific antibody, such as a bispecific antibody or a trispecific antibody.

In some embodiments, there is provided a multispecific antibody comprising a first polypeptide comprising a first CH3 domain and a first target binding moiety (TBM), a second polypeptide comprising a second CH3 domain and a second TBM, wherein the first CH3 domain and the second CH3 domain comprise any one or combination of the engineered disulfide bonds or salt bridges described herein, wherein the first TBM specifically binds to a first target, and wherein the second TBM specifically binds to a second target that is different from the first target. In some embodiments, the TBM is an antigen-binding domain. In some embodiments, the TBM is a scFv or a VHH. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution.

In some embodiments, there is provided a multispecific antibody comprising a first CH3 domain, a second polypeptide comprising a second CH3 domain, a third polypeptide, and a fourth polypeptide, wherein the first CH3 domain and the second CH3 domain comprise any one or combination of the engineered disulfide bonds or salt bridges described herein, wherein the first polypeptide is a first antibody heavy chain, the second polypeptide is a second antibody heavy chain, the third polypeptide is a first antibody light chain, and the fourth polypeptide is a second antibody light chain, wherein the first polypeptide and the third polypeptide associate to form a first antigen binding site that specifically binds a first target, and the second polypeptide and the fourth polypeptide associate to form a second antigen binding site that specifically binds a second target that is different from the first target. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D. K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution.

In some embodiments, there is provided a multispecific antibody comprising a first polypeptide comprising a first CH3 domain, a second polypeptide comprising a second CH3 domain, a third polypeptide, and a fourth polypeptide, wherein the first CH3 domain and the second CH3 domain comprise any one or combination of the engineered disulfide bonds or salt bridges described herein, wherein:

(i) the first polypeptide comprises a structure represented by the formula:

VH1-CH1-hinge-CH2-first CH3;

(ii) the second polypeptide comprises a structure represented by the formula:

VH2-CH1-hinge-CH2-second CH3;

(iii) the third polypeptide comprises a structure represented by the formula:

VL1-CL; and

(iv) the fourth polypeptide comprises a structure represented by the formula:

VL2-CL;

wherein:

VL1 is a first immunoglobulin light chain variable domain;

VH1 is a first immunoglobulin heavy chain variable domain;

VL2 is a second immunoglobulin light chain variable domain;

VH2 is a second immunoglobulin heavy chain variable domain;

CL is an immunoglobulin light chain constant domain;

CH1 is an immunoglobulin heavy chain constant domain 1;

CH2 is an immunoglobulin heavy chain constant domain 2:
hinge is an immunoglobulin hinge region connecting the CH1 and CH2 domains;
   wherein VL1 and VH1 associate to form a first Fv that specifically binds to a first target; and wherein VL2 and VH2 associate to form a second Fv that specifically binds to a second target. In some embodiments, VL1 is identical to VL2 (e.g., the multispecific antibody is a common light chain antibody). In some embodiments, VL1 is different from VL2. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution. In some embodiments, the first target is PDL1 and the second target is CD137, or the first target is CD137 and the second target is PDL1.

In some embodiments, there is provided a multispecific antibody comprising a first polypeptide comprising a first CH3 domain, a second polypeptide comprising a second CH3 domain, a third polypeptide, and a fourth polypeptide, wherein the first CH3 domain and the second CH3 domain comprise any one or combination of the engineered disulfide bonds or salt bridges described herein, wherein:

(i) the first polypeptide comprises a structure represented by the formula:

VH1-CH1-hinge-CH2-first CH3-L1-scFv1;

(ii) the second polypeptide comprises a structure represented by the formula:

VH2-CH1-hinge-CH2-second CH3-L2-scFv2;

(iii) the third polypeptide comprises a structure represented by the formula:

VL1-CL; and

(iv) the fourth polypeptide comprises a structure represented by the formula:

VL2-CL;

wherein:

VL1 is a first immunoglobulin light chain variable domain;

VH1 is a first immunoglobulin heavy chain variable domain;

VL2 is a second immunoglobulin light chain variable domain;

VH2 is a second immunoglobulin heavy chain variable domain;

scFv1 is a first single-chain variable fragment;

scFv2 is a second single-chain variable fragment;

CL is an immunoglobulin light chain constant domain;

CH1 is an immunoglobulin heavy chain constant domain 1;

CH2 is an immunoglobulin heavy chain constant domain 2:
hinge is an immunoglobulin hinge region connecting the CH1 and CH2 domains; and L1 and L2 is each independently a bond or a peptide linker;
   wherein VL I and VH1 associate to form a first Fv that specifically binds to a first target; wherein VL2 and VH2 associate to form a second Fv that specifically binds to a second target; wherein scFv1 specifically binds to a third target; and wherein scFv2 specifically binds to a fourth target. In some embodiments, the heterodimeric protein is a bispecific antibody, wherein the first target and the second target are the same, and the third target and the fourth target are the same. In some embodiments, the heterodimeric protein is a trispecific antibody, wherein the third target and the fourth target are the same or the first target and the second target are the same. In some embodiments, the heterodimeric protein is a tetraspecific antibody. In some embodiments, VL1 is identical to VL2 (e.g., the multispecific antibody is a common light chain antibody). In some embodiments, VL1 is different from VL2. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D. N390C and K439D substitutions, or the first CH3 domain comprises L351D, K370D, N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution.

In some embodiments, there is provided a multispecific antibody comprising a first polypeptide comprising a first CH3 domain, a second polypeptide comprising a second CH3 domain and a first target binding moiety (TBM) that specifically binds a first target, and a third polypeptide, wherein the first CH3 domain and the second CH3 domain comprise any one or combination of the engineered disulfide bonds or salt bridges described herein, wherein the first polypeptide and the third polypeptide associate to form a second antigen binding site that specifically binds a second target. In some embodiments, the first target binding moiety (TBM) is a scFv or a VHH. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution. In some embodiments, the first target is PDL1 and the second target is CD137. In some embodiments, the first target is CD137 and the second target is PDL1. In some embodiments, the first target is CD137 and the second target is CTLA4. In some embodiments, the first target is CTLA4 and the second target is PDL1.

In some embodiments, there is provided a multispecific antibody comprising a first polypeptide comprising a first CH3 domain, a second polypeptide, and a third polypeptide, wherein the first CH3 domain and the second CH3 domain comprise any one or combination of the engineered disulfide bonds or salt bridges described herein, wherein:

(i) the first polypeptide comprises a structure represented by the formula:

VH-CH1-hinge-CH2-first CH3;

(ii) the second polypeptide comprises a structure represented by the formula:

scFv-hinge-CH2-second CH3; and

(iii) the third polypeptide comprises a structure represented by the formula

VL-CL;

wherein:

VL is an immunoglobulin light chain variable domain;

VH is an immunoglobulin heavy chain variable domain;

scFv is a single-chain variable fragment;

CL is an immunoglobulin light chain constant domain;

CH1 is an immunoglobulin heavy chain constant domain 1;

CH2 is an immunoglobulin heavy chain constant domain 2; and

hinge is an immunoglobulin hinge region connecting the CH1 and CH2 domains:

wherein VL and VH associate to form an Fv that specifically binds to a first target; and wherein the scFv specifically binds to a second target. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D. N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution. In some embodiments, the scFv is linked to the hinge in the second polypeptide via a linker, such as a peptide linker comprising the amino acid sequence of SEQ ID NO: 80 or 81. In some embodiments, the first target is CD3 and the second target is a tumor antigen (e.g., HER2). In some embodiments, the first target is a tumor antigen (e.g., HER2) and the second target is CD3.

In some embodiments, the multispecific antibody comprises one or more antibody constant regions. In some embodiments, the human heavy chain constant region is of an isotype selected from IgA, IgG, and IgD. In some embodiments, the human light chain constant region is of an isotype selected from κ and λ. In some embodiments, the multispecific antibody comprises a human IgG constant region. In some embodiments, the multispecific antibody comprises a human IgG4 heavy chain constant region. In some embodiments, the multispecific antibody comprises a human IgG1 heavy chain constant region. In some such embodiments, the multispecific antibody comprises an S228P mutation in the human IgG4 constant region. In some embodiments, the first polypeptide and the second polypeptide further comprises S228P substitution.

Whether or not effector function is desirable may depend on the particular method of treatment intended for a multispecific antibody. In some embodiments, when effector function is desirable, a multispecific antibody comprising a human IgG1 heavy chain constant region or a human IgG3 heavy chain constant region is selected. In some embodiments, when effector function is not desirable, a multispecific antibody comprising a human IgG4 or IgG2 heavy chain constant region is selected. In some embodiments, the multispecific antibody comprises a human IgG1 heavy chain constant region comprising one or more mutations that reduces effector function. In some embodiments, the multispecific antibody comprises an IgG1 heavy chain constant region comprising an N297A substitution. In some embodiments, the first polypeptide and the second polypeptide further comprises N297A substitution.

Any of the multispecific antibodies described herein can specifically bind at least two different targets, or epitopes. The at least two different epitopes recognized can be located on the same antigen, or on different antigens. In some embodiments, the antigens are cell surface molecules. In some embodiments, the antigens are extracellular molecules.

In some embodiments, the first target, second target, third target and/or fourth target is a cell surface antigen. In some embodiments, the cell surface antigen is an antigen on immune effector cells, such as T cells (e.g., helper T cells, cytotoxic T cells, memory T cells, etc.), B cells, macrophages, and Natural Killer (NK) cells. In some embodiments, the cell surface antigen is a T cell surface antigen, such as CD3.

In some embodiments, the cell surface antigen is a tumor antigen. Tumor antigens are proteins that are produced by tumor cells that can elicit an immune response, particularly T-cell mediated immune responses. In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the first, second, third and/or fourth target is an immune checkpoint molecule. In some embodiments, the immune checkpoint molecule is a stimulatory immune checkpoint molecule. Exemplary stimulatory immune checkpoint molecules include, but are not limited to, CD28, OX40, ICOS, GITR, 4-1BB, CD27, CD40, CD3, HVEM, and TCR (e.g., MHC class I or class 11 molecules). In some embodiments, the immune checkpoint molecule is an inhibitory immune checkpoint molecule. Exemplary inhibitory immune checkpoint molecules include, but are not limited to, CTLA-4, TIM-3, A2a Receptor, LAG-3, BTLA, KIR, PD-1, IDO, CD47, and ligands thereof such as B7.1, B7.2, PDL1, PD-L2, HVEM, B7-H4, NKTR-218, and SIRP-alpha receptor.

### Target Binding Moiety (TBM)

In some embodiments, the target binding moiety (TBM) comprises an antibody light chain variable region (VL) and/or an antibody heavy chain variable region (VH). In some embodiments, the TBM comprises a VL. In some embodiments, the TBM comprises a VH. In some embodiments, a TBM comprises a VL and/or a VH specificity for any target of interest, including, for example, CTLA4, CD137, PD1, PDL1, PDL2, LAG3, TIM3, B7-H3, OX40, CD3, CD19, CD20, CD40, CD95, CD120a, BTLA, VISTA, ICOS, BCMA, HER1, HER2, HER3, and/or B7-H4.

In some embodiments, the TBM is an antigen binding fragment, including, but not limited to: (i) a Fab fragment, which is a monovalent fragment consisting of the VL, VH, CL and CH1 domains; (ii) a F(ab′)₂ fragment, which is a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (Ward et al., (1989) Nature 341:544-546), which consists of a VH domain; (vi) an isolated CDR, and (vii) single chain antibody (scFv), which is a polypeptide comprising a VL region of an antibody linked to a VH region of an antibody (see e.g., Bird et al. (1988) Science 242:423-426; Huston et al. (1988) Proc. Natl. Acad. Sci. USA 85:5879-5883).

In some embodiments, the TBM is a scFv comprising from the N-terminus to the C-terminus: VL-L1-VH, wherein L1 is a peptide linker. In some embodiments, the TBM is a scFv comprising from the N-terminus to the C-terminus: VH-L1-VL, wherein L1 is a peptide linker. In some embodiment, L1 comprises the amino acid sequence of SEQ ID NO: 82. In some embodiments, the TBM is a scFv comprising an engineered disulfide bond between VH and VL, such as between C44 of VH and C100 of VL, wherein the numbering is based on Kabat numbering. In some embodiments, the scFv comprises a first cysteine residue at position 44 in the VH and a second cysteine residue at position 100 in the VL, wherein the first cysteine residue and the second cysteine residue form a disulfide bond, and wherein the numbering is based on Kabat numbering.

In some embodiments, the TBM comprises a full-length antibody light chain and/or a full-length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or IgG4 subclass. An antibody heavy chain described herein may be converted from one class or subclass to another class or subclass using methods known in the art.

The multispecific antibodies described herein may comprise TBMs derived from any suitable antibodies targeting antigens of interest. The TBMs described herein may incorporate any of the CDR sequences (e.g. one, two, or three of the heavy chain variable region CDR sequences, and/or one, two, or three of the light chain variable region CDR sequences), heavy chain variable region sequences, and/or light chain variable region sequences of any of the antibodies described in WO2019/036856, WO2019/036842, WO2019/036855, WO2019148444, WO2019185035, WO2019036855, which are incorporated herein by reference in their entirety. Table C below shows antibody CDRs, VH, VL, scFv sequences of exemplary TBMs thereof described herein.

TABLE C
Exemplary multispecific antibodies

| SEQ ID NO | Antibody sequence | Amino acid sequence (underlined are CDR sequences) |
|---|---|---|
| 37 | PDL1 CDR-H1 | YSISSGYYWG |
| 38 | PDL1 CDR-H2 | GIIYPSGGGTNYAQKFQG |
| 39 | PDL1 CDR-H3 | GGGLGFDY |
| 40 | PDL1 CDR-L1 | RASQSIPSFLN |
| 41 | PDL1 CDR-L2 | AASSLQS |
| 42 | PDL1 CDR-L3 | QHYISWPRQFT |
| 43 | PDL1 VH | EVQLVESGGGLVQPGGSLRLSCAASG [symbol] [symbol] WIRQAPGKGLEWI |
| | | [symbol] [symbol] RVTISRDNSKNTLYLQLNSLRAEDTAVYYCA |
| | | R [symbol] WGQGTLVTVSS |
| 44 | PDL1 VL | DIQLTQSPSSLSASVGDRVTITC [symbol] WYQQKPGKAPKLLIY |
| | | [symbol] GVPSRFSGSGSGTDFTLTISSLQPEDFATYYC [symbol] |
| | | [symbol] FGQGTKVEIKR |
| 45 | CD137 CDR-H1 | FSLSTGGVGVG |
| 46 | CD137 CDR-H2 | ALIDWADDKYYSPSLKS |
| 47 | CD137 CDR-H3 | GGSDTVIGDWFAY |
| 48 | CD137 CDR-L1 | RASQSIGSYLA |
| 49 | CD137 CDR-L2 | DASNLET |
| 50 | CD137 CDR-L3 | QQGYYLWT |
| 51 | CD137 VH | EVQLVESGGGLVQPGGSLRLSCAASG [symbol] [symbol] WIRQAPGKGLEW |
| | | L [symbol] [symbol] RLTISRDNSKNTLYLQLNSLRAEDTAVYYCA |
| | | R [symbol] [symbol] WGQGTLVTVSS |
| 52 | CD137 VL | DIQLTQSPSSLSASVGDRVTITC [symbol] WYQQKPGKAPKLLIY |
| | | [symbol] GVPSRFSGSGSGTDFTLTISSLQPEDFATYYC [symbol] |
| | | FGQGTKVEIKR |
| 53 | CTLA4 CDR-H1 | YSISSGYHWS |
| 54 | CTLA4 CDR-H2 | ARIDWDDDKYYSTSLKS |
| 55 | CTLA4 CDR-H3 | SYVYFDY |
| 56 | CTLA4 CDR-L1 | RASQSVRGRFLA |
| 57 | CTLA4 CDR-L2 | DASNRAT |
| 58 | CTLA4 CDR-L3 | QQSSSWPPT |
| 59 | CTLA4 VH | EVQLVESGGGLVQPGGSLRLSCAASG [symbol] [symbol] WIRQAPGKGLEWL |
| | | [symbol] [symbol] RLTISRDNSKNTLYLQLNSLRAEDTAVYYCAR |
| | | [symbol] WGQGTLVTVSS |
| 60 | CTLA4 VL | DIQLTQSPSSLSASVGDRVTITC [symbol] [symbol] WYQQKPGKAPKLLI |
| | | Y [symbol] GIPSRFSGSGSGTDFTLTISSLQPEDFATYYC [symbol] |
| | | FGQGTKVEIKR |
| 61 | CD3 CDR-H1 | FTFNTYAVIN |
| 62 | CD3 CDR-H2 | GRIRSKYNNYATYYADSVKG |
| 63 | CD3 CDR-H3 | HGNFGNSYVSWFAY |
| 64 | CD3 CDR-L1 | GSSTGAVTTSNYAN |
| 65 | CD3 CDR-L2 | GTNKRAP |
| 66 | CD3 CDR-L3 | WYSNLWV |
| 67 | CD3 VH | EVQLVESGGGLVQPGGSLRLSCAASG [symbol] WVRQAPGKGLEWV |
| | | [symbol] [symbol] RFTISRDDSKNTLYLQMNSLRAEDTAVY |
| | | YCVR [symbol] [symbol] WGQGTLVTVSS |
| 68 | CD3 VL | QAVVTQEPSLTVSPGGTVTLTC [symbol] [symbol] WVQQKPGQAPRGL |
| | | IG [symbol] GVPARFSGSLLGGKAALTLSGAQPEDEAEYYCAL |
| | | [symbol] FGGGTKLTVL |
| 69 | HER2 CDR-H1 | FNIKDTYIH |
| 70 | HER2 CDR-H2 | ARIYPTNGYTRYADSVKG |
| 71 | HER2 CDR-H3 | WGGDGFYAMDY |
| 72 | HER2 CDR-L1 | RASQDVNTAVA |
| 73 | HER2 CDR-L2 | SASFLYS |
| 74 | HER2 CDR-L3 | QQHYTTPPT |
| 75 | HER2 VH | EVQLVESGGGLVQPGGSLRLSCAASG [symbol] WVRQAPGKGLEWV |
| | | [symbol] [symbol] RFTISADTSKNTAYLQMNSLRAEDTAVYYCS |
| | | R [symbol] WGQGTLVTVSS |
| 76 | HER2 VL | DIQMTQSPSSLSASVGDRVTITC [symbol] WYQQKPGKAPKLLIY |
| | | [symbol] GVPSRFSGSRSGTDFTLTISSLQPEDFATYYC [symbol] |
| | | FGQGTKVEIKR |
| 77 | anti-PDL1 scFv | EVQLVESGGGLVQPGGSLRLSCAASGYSISSGYYWGWIRQAPGKGLEWIG |
| | | IIYPSGGGTNYAQKIQGRVTISRDNSKNTLYLQLNSLRAEDTAVYYCARG |
| | | GGLGFDYWGQGTLVTVSSGGGGSGGGGSGGGGSGGGGSDIQLTQSPSSLS |
| | | ASVGDRVTITCRASQSIPSFLNWYQQKPGKAPKLLIYAASSLQSGVPSRF |
| | | SGSGSGTDFTLTISSLQPEDFATYYCQHYISWPRQFTFGQGTKVEIKR |
| 78 | anti-CD137 scFv | EVQLVESGGGLVQPGGSLRLSCAASGFSLSTGGVGVGWIRQAPGKGLEWL |
| | | ALIDWADDKYYSPSLKSRLTISRDNSKNTLYLQLNSLRAEDTAVYYCARG |
| | | GSDTVIGDWFAYWGQGTLVTVSSGGGGSGGGGSGGGGSGGGGSDIQLFQS |
| | | PSSLSASVGDRVTITCRASQSIGSYLAWYQQKPGKAPKLLIYDASNLETG |
| | | VPSRFSGSGSGTDFTLTISSLQPEDFATYYCQQGYYLWTFGQGTKVEIKR |
| 79 | anti-CD3 scFv | QAVVTQEPSLTVSPGGTVTLTCGSSTGAVTTSNYANWVQQKPGQAPRGLI |
| | | GGTNKRAPGVPARFSGSLLGGKAALTLSGAQPEDEAEYYCALWYSNLWVF |
| | | GGGTKLTVLRGGGGSGGGGSGGGGSGGGGSEVQLVESGGGLVQPGGSLRL |
| | | SCAASGFTFNTYAMNWVRQAPGKGLEWVGRIRSKYNNYATYYADSVKGRF |
| | | TISRDDSKNTLYLQMNSLRAEDTAVYYCVRHGNFGNSYVSWFAYWGQGTL |
| | | VTVSS |
| 83 | anti-CTLA4 scFv | EVQLVESGGGLVQPGGSLRLSCAASGYSISSGYHWSWIRQAPGKGLEWLA |
| | | RIDWDDDKYYSTSLKSRLTISRDNSKNTLYLQLNSLRAEDTAVYYCARSY |
| | | VYFDYWGQGTLVTVSSGGGGSGGGGSGGGGSGGGGSDIQLTQSPSSLSAS |
| | | VGDRVTITCRASQSVRGRFLAWYQQKPGKAPKLLIYDASNRATGIPSRFS |
| | | GSGSGTDFTLTISSLQPEDFATYYCQQSSSWPPTFGQGTKVEIKR |

In some embodiments, the TBM is an anti-PDL1 antibody or antigen binding domain thereof, including, e.g., a VH, VL, scFv, light chain, or heavy chain (such as IgG1, IgG2 IgG4). Any of the known anti-PD-L1 antibodies may be used in the present invention, see, for example, U.S. Pat. Nos. 7,943,743, 7,722,868, 8,217,149, 8,383,796, 8,552,154, and 9,102,725; and U.S. Patent Application Publication Nos. US20140341917, and US20150203580; and International Patent Application No. PCT/US2001/020964. Exemplary anti-PD-L1 antibodies include, but are not limited to, BMS935559 (also known as MDX-1105), MPDL3280A, MEDI4736, Avelumab (also known as MSB0010718C), KY-1003, MCLA-145, RG7446 (also known as atezolizumab), SHR-1316, STI-3031, ZKAB001, TQB2450, LY3300054 and STI-A1010.

In some embodiments, the TBM comprises a VH comprising an antibody heavy chain complementarity determining region (CDR-H) 1 comprising the amino acid sequence of SEQ ID NO: 37, a CDR-H2 comprising the amino acid sequence of SEQ ID NO: 38, and/or a CDR-H3 comprising the amino acid sequence of SEQ ID NO: 39. In some embodiments, the TBM comprises a VL comprising an antibody light chain complementarity determining region (CDR-L) I comprising the amino acid sequence of SEQ ID NO: 40, a CDR-L2 comprising the amino acid sequence of SEQ ID NO: 41, and/or a CDR-L3 comprising the amino acid sequence of SEQ ID NO: 42. In some embodiments, the TBM comprises a VH comprising the amino acid sequence of SEQ ID NO: 43. In some embodiments, the TBM comprises a VL comprising the amino acid sequence of SEQ ID NO: 44. In some embodiments, the TBM comprises a scFv comprising the amino acid sequence of SEQ ID NO: 77.

In some embodiments, the TBM is an anti-CD137 antibody or antigen binding domain thereof, including, e.g., a VH, VL, scFv, light chain, or heavy chain (such as IgG1, IgG2, IgG4). Any of the known anti-CD137 antibodies may be used in the present invention, for example, see, WO2016/134358. Exemplary anti-CD137 antibodies include, but are not limited to, Urelumab (also known as BMS-663513), Utomilumab (also known as PF-05082566), CTX-471, ATOR-1017 and AGEN2373.

In some embodiments, the TBM comprises a VH comprising an CDR-H1 comprising the amino acid sequence of SEQ ID NO: 45, a CDR-H2 comprising the amino acid sequence of SEQ ID NO: 46, and/or a CDR-H3 comprising the amino acid sequence of SEQ ID NO: 47. In some embodiments, the TBM comprises a VL comprising a CDR-L1 comprising the amino acid sequence of SEQ ID NO: 48, a CDR-L2 comprising the amino acid sequence of SEQ ID NO: 49, and/or a CDR-L3 comprising the amino acid sequence of SEQ ID NO: 50. In some embodiments, the TBM comprises a VH comprising the amino acid sequence of SEQ ID NO: 51. In some embodiments, the TBM comprises a VL comprising the amino acid sequence of SEQ ID NO: 52. In some embodiments, the TBM comprises a scFv comprising the amino acid sequence of SEQ ID NO: 78.

In some embodiments, the TBM is an anti-CTLA4 antibody or antigen binding domain thereof, including, e.g., a VH, VL, scFv, light chain, or heavy chain (such as IgG1, IgG2, IgG4). Any of the known anti-CTLA4 antibodies may be used in the present invention, including but not limited to, Ipilimumab (see U.S. Pat. Nos. 6,984,720, 7,452,535, 7,605,238, 8,017,114 and 8,142,778), Tremilimumab (see U.S. Pat. Nos. 6,68,736, 7,109,003, 7,132,281, 7,411,057, 7.807,797, 7,824,679 and 8,143,379) and other anti-CTLA-4 antibodies, such as single chain antibodies (e.g., see U.S. Pat. Nos. 5,811,097, 6,051,227 and 7,229,628, US Patent Publication No. US20110044953, US Patent Publication No. US2018037654, US Patent Publication No. US2009025274. US Patent Publication No. US2019127468, International Patent Publication No. WO2019/152413, International Patent Publication No. WO2018209701, International Patent Publication No. WO2018/202649 and International Patent Publication No. WO2019/152423). Other exemplary anti-CTLA-4 antibodies include RG2077, ONC-392, CS1002, BCD-145, IBI310. AGEN1884, AGEN1181 and AGEN2041.

In some embodiments, the TBM comprises a VH comprising an CDR-HI comprising the amino acid sequence of SEQ ID NO: 53, a CDR-H2 comprising the amino acid sequence of SEQ ID NO: 54, and/or a CDR-H3 comprising the amino acid sequence of SEQ ID NO: 55. In some embodiments, the TBM comprises a VL comprising a CDR-L1 comprising the amino acid sequence of SEQ ID NO: 56, a CDR-L2 comprising the amino acid sequence of SEQ ID NO: 57, and/or a CDR-L3 comprising the amino acid sequence of SEQ ID NO: 58. In some embodiments, the TBM comprises a VH comprising the amino acid sequence of SEQ ID NO: 59. In some embodiments, the TBM comprises a VL comprising the amino acid sequence of SEQ ID NO: 60. In some embodiments, the TBM comprises a scFv comprising the amino acid sequence of SEQ ID NO: 83.

In some embodiments, the TBM is an anti-CD3 antibody or antigen binding domain thereof, including, e.g., a VH, VL, scFv, light chain, or heavy chain (such as IgG1, IgG2, IgG4). Any of the known anti-CD3 antibodies may be used in the present invention, including but not limited to, the Cris-7 monoclonal antibody (Reinherz, E. L. et al. (eds.), Leukocyte typing II, Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140: 3766), Otelixizumab and Foralumab. Further CD3 binding molecules contemplated herein include UCHT-1 (Beverley, P C and Callard, R. E. (1981) Eur. J. Immunol. 11: 329-334, SP34 (Silvana et. al. (1985) The EMBO Journal. 4:337-344) and CD3 binding molecules described in WO2004/106380; WO2010/037838; WO2008/119567; WO2007/042261: WO2010/0150918; WO2018/052503; WO2016/204966.

In some embodiments, the TBM comprises a VH comprising an CDR-HI comprising the amino acid sequence of SEQ ID NO: 61, a CDR-H2 comprising the amino acid sequence of SEQ ID NO: 62, and/or a CDR-H3 comprising the amino acid sequence of SEQ ID NO: 63. In some embodiments, the TBM comprises a VL comprising a CDR-L1 comprising the amino acid sequence of SEQ ID NO: 64, a CDR-L2 comprising the amino acid sequence of SEQ ID NO: 65, and/or a CDR-L3 comprising the amino acid sequence of SEQ ID NO: 66. In some embodiments, the TBM comprises a VH comprising the amino acid sequence of SEQ ID NO: 67. In some embodiments, the TBM comprises a VL comprising the amino acid sequence of SEQ ID NO: 68. In some embodiments, the TBM comprises a scFv comprising the amino acid sequence of SEQ ID NO: 79.

In some embodiments, the TBM is an anti-HER2 antibody or antigen binding domain thereof, including, e.g., a VH, VL, scFv, light chain, or heavy chain (such as IgG1, IgG2, IgG4). Any of the known anti-HER2 antibodies may be used in the present invention, including but not limited to, Herceptin (1998, Cancer Res 58 (13):2825-2831), MDXH210 (Schwaab et al., 2001, Journal of Immunotherapy, 24(1):79-87), Disitamab (Toxicol Lett. 2019. S0378-4274(19)30421-7), Pertuzumab (Agus D B, Gordon M S, Taylor C, et al. J Clin Oncol. 2005; 23(11):2534-2543).

In some embodiments, the TBM comprises a VH comprising an CDR-H1 comprising the amino acid sequence of SEQ ID NO: 69, a CDR-H2 comprising the amino acid sequence of SEQ ID NO: 70, and/or a CDR-H3 comprising the amino acid sequence of SEQ ID NO: 71. In some embodiments, the TBM comprises a VL comprising a CDR-L1 comprising the amino acid sequence of SEQ ID NO: 72, a CDR-L2 comprising the amino acid sequence of SEQ ID NO: 73, and/or a CDR-L3 comprising the amino acid sequence of SEQ ID NO: 74. In some embodiments, the TBM comprises a VH comprising the amino acid sequence of SEQ ID NO: 75. In some embodiments, the TBM comprises a VL comprising the amino acid sequence of SEQ ID NO: 76.

The term “PDL1” as used in the present application includes human PDL1 (e.g., UniProt accession number Q9NZQ7), as well as variants, isoforms, and species homologs thereof (e.g., mouse PDL1 (UniProt accession number Q9EP73), rat PDL1 (UniProt accession number P52944), dog PDL1 (UniProt accession number E2RKZ5), cynomolgus monkey PDL1, etc.).

The term “CTLA4” as used in the present application includes human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1 A7), dog CTLA4 (UniProt accession number Q9XSI1), cynomolgus monkey CTLA4 (UniProt accession number G7PL88), etc.).

The term “CD137” as used in the present application includes the human CD137 (e.g., GenBank Accession No. NM_001561; NP_001552), as well as variants, isoforms, and species homologs thereof (e.g., mouse CD137 (GenBank Gene ID 21942), rat CD137 (GenBank Gene ID 500590), dog CD137 (GenBank Gene ID 608274), cynomolgus monkey CTLA4 (GenBank Gene ID 102127961), etc.).

The term “CD3” is known in the art as a multi-protein complex of six chains (see, Abbas and Lichtman, 2003; Janeway et al., p172 and 178, 1999). In mammals, the complex comprises a CD3 gamma chain, a CD3 delta chain, two CD3 epsilon chains, and a homodimer of CD3 zeta chains. The CD3 gamma, CD3 delta, and CD3 epsilon chains are highly related cell surface proteins of the immunoglobulin superfamily containing a single immunoglobulin domain. The transmembrane regions of the CD3 gamma, CD3 delta, and CD3 epsilon chains are negatively charged, which is a characteristic that allows these chains to associate with the positively charged T cell receptor chains. The intracellular tails of the CD3 gamma, CD3 delta, and CD3 epsilon chains each contain a single conserved motif known as an immunoreceptor tyrosine-based activation motif or ITAM, whereas each CD3 zeta chain has three. Without being bound by theory, it is believed the ITAMs are important for the signaling capacity of a TCR complex. CD3 as used herein may be from various animal species, including human, primate, mouse, rat, or other mammals. For example, CD3 as used herein includes human CD3e (i.e., CD3 epsilon; e.g., UniProt accession number P07766), as well as variants, isoforms, and species homologs thereof (e.g., mouse CD3e (UniProt accession number P22646), rat CD3e (UniProt accession number A0A0G2K986), dog CD3e (UniProt accession number P27597), and cynomolgus monkey CD3e (UniProt accession number Q95LI5)).

The term “HER2” as used in the present application includes human HER2 (e.g., UniProt accession number P04626), as well as variants, isoforms, and species homologs thereof (e.g., mouse HER2 (UniProt accession number P70424), rat HER2 (UniProt accession number P06494), dog HER2, cynomolgus monkey HER2. HER2 is also known as ERBB2.

The TBMs described herein may bind a human target (e.g., PDL1, CTLA4, CD137, CD3 or HER2). In some cases, a TBM may be completely specific for the human target and may not exhibit species or other types of cross-reactivity. In other cases, a TBM also binds targets from species other than human.

### Linker

The multispecific antibodies described herein may comprise one or more linkers (e.g., L1, L2, L3, etc.) disposed between the various regions in the polypeptides.

Any suitable linker (e.g., a flexible linker) known in the art may be used, including, for example: glycine polymers (G)n, where n is an integer of at least 1 (e.g., at least one, at least 2, at least 3, at least 4, at least 5, at least 6, at least 7, at least 8, at least 9, at least 10, etc.); glycine-serine polymers (GS)n, where n is an integer of at least 1 (e.g., at least one, at least 2, at least 3, at least 4, at least 5, at least 6, at least 7, at least 8, at least 9, at least 10, etc.) such as SGGGS (SEQ ID NO: 80), GGGSGGGGS (SEQ ID NO: 81), (G₄S)₄ (SEQ ID NO: 82), GGGGS (SEQ ID NO: 130), SGGS (SEQ ID NO: 131), GGSG (SEQ ID NO: 132), GGSGG (SEQ ID NO: 133), GSGSG (SEQ ID NO: 134), GSGGG (SEQ ID NO: 135), GGGSG (SEQ ID NO: 136), and/or GSSSG (SEQ ID NO: 137)); glycine-alanine polymers: alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length.

### Exemplary Multispecific Antibodies

Exemplary multispecific antibodies described herein include, but are not limited to, bispecific antibodies targeting PDL1 and CD137 (e.g., PDL1×CD137 and CD137×PDL1 antibodies), bispecific antibodies targeting CD137 and CTLA4 (e.g., CD137×CTLA4 antibody), bispecific T-cell engagers (BiTE) targeting CD3 and a cell surface antigen, and trispecific antibodies targeting PDL1, CD137 and CTLA4 (also referred herein as PDL1×CD137×CTLA4 antibody). In some embodiments, the multispecific antibody comprises CH3 domains or Fc regions comprising any one or combination of engineered disulfide bonds and/or salt bridges described herein. In some embodiments, the multispecific antibody does not comprise CH3 domains or Fc regions comprising any one or combination of engineered disulfide bonds and/or salt bridges described herein.

In some embodiments, there is provided a bispecific antibody targeting PDL1 and CD137, comprising a first polypeptide and a second polypeptide, wherein:

(i) the first polypeptide comprises a structure represented by the formula:

VH1-CH1-hinge-CH2-first CH3-L1-scFv1; and

(ii) the second polypeptide comprises a structure represented by the formula:

VL-CL;

wherein:

VL is an immunoglobulin light chain variable domain;

VH is an immunoglobulin heavy chain variable domain;

scFv is a single-chain variable fragment:

CL is an immunoglobulin light chain constant domain;

CH1 is an immunoglobulin heavy chain constant domain 1;

CH2 is an immunoglobulin heavy chain constant domain 2:
hinge is an immunoglobulin hinge region connecting the CH1 and CH2 domains;
and L1 is a bond or a peptide linker;
   wherein VL and VH associate to form an Fv that specifically binds to a CD137; wherein the scFv specifically binds to PDL1. In some embodiments, the scFv comprises a VH comprising a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and/or a VL comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the scFv comprises a VH comprising the amino acid sequence of SEQ ID NO: 43, and/or a VL comprising the amino acid sequence of SEQ ID NO: 44. In some embodiments, the scFv comprises from the N-terminus to the C-terminus: VH-L1-VL, wherein L1 is a peptide linker. In some embodiment, L1 comprises the amino acid sequence of SEQ ID NO: 82. In some embodiments, the scFv comprises a first cysteine residue at position 44 in the VH and a second cysteine residue at position 100 in the VL, wherein the first cysteine residue and the second cysteine residue form a disulfide bond, and wherein the numbering is based on Kabat numbering. In some embodiments, the scFv comprises the amino acid sequence of SEQ ID NO: 77. In some embodiments, the Fv comprises a VH comprising an CDR-HI comprising the amino acid sequence of SEQ ID NO: 45, a CDR-H2 comprising the amino acid sequence of SEQ ID NO: 46, and a CDR-H3 comprising the amino acid sequence of SEQ ID NO: 47; and/or a VL comprising a CDR-L1 comprising the amino acid sequence of SEQ ID NO: 48, a CDR-L2 comprising the amino acid sequence of SEQ ID NO: 49, and a CDR-L3 comprising the amino acid sequence of SEQ ID NO: 50. In some embodiments, the Fv comprises a VH comprising the amino acid sequence of SEQ ID NO: 51 and/or a VL comprising the amino acid sequence of SEQ ID NO: 52. In some embodiments, the first CH3 domain comprises N390C substitution and the second CH3 domain comprises S400C substitution, or the first CH3 domain comprises S400C substitution and the second CH3 domain comprises N390C substitution. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution. In some embodiments, the scFv is linked to the hinge in the second polypeptide via a linker, such as a peptide linker comprising the amino acid sequence of SEQ ID NO: 80 or 81.

In some embodiments, there is provided a bispecific antibody comprising a first polypeptide comprising the amino acid sequence of SEQ ID NO: 96, and a second polypeptide comprising the amino acid sequence of SEQ ID NO: 97. In some embodiments, there is provided a bispecific antibody comprising a first polypeptide comprising the amino acid sequence of SEQ ID NO: 98, and a second polypeptide comprising the amino acid sequence of SEQ ID NO: 99. In some embodiments, there is provided a bispecific antibody comprising a first polypeptide comprising the amino acid sequence of SEQ ID NO: 100, and a second polypeptide comprising the amino acid sequence of SEQ ID NO: 101. In some embodiments, there is provided a bispecific antibody comprising a first polypeptide comprising the amino acid sequence of SEQ ID NO: 102, and a second polypeptide comprising the amino acid sequence of SEQ ID NO: 103. In some embodiments, there is provided a bispecific antibody comprising a first polypeptide comprising the amino acid sequence of SEQ ID NO: 104, and a second polypeptide comprising the amino acid sequence of SEQ ID NO: 105. In some embodiments, there is provided a bispecific antibody comprising a first polypeptide comprising the amino acid sequence of SEQ ID NO: 106, and a second polypeptide comprising the amino acid sequence of SEQ ID NO: 107. In some embodiments, there is provided a bispecific antibody comprising a first polypeptide comprising the amino acid sequence of SEQ ID NO: 108, and a second polypeptide comprising the amino acid sequence of SEQ ID NO: 109. In some embodiments, there is provided a bispecific antibody comprising a first polypeptide comprising the amino acid sequence of SEQ ID NO: 110, and a second polypeptide comprising the amino acid sequence of SEQ ID NO: 111.

In some embodiments, there is provided a bispecific antibody targeting PDL1 and CD137, comprising a first polypeptide and a second polypeptide, wherein:

(i) the first polypeptide comprises a structure represented by the formula:

VH1-CH1-hinge-CH2-first CH3-L1-scFv1; and

(ii) the second polypeptide comprises a structure represented by the formula:

VL-CL;

wherein:

VL is an immunoglobulin light chain variable domain;

VH is an immunoglobulin heavy chain variable domain;

scFv is a single-chain variable fragment:

CL is an immunoglobulin light chain constant domain;

CH1 is an immunoglobulin heavy chain constant domain 1;

CH2 is an immunoglobulin heavy chain constant domain 2:
hinge is an immunoglobulin hinge region connecting the CH1 and CH2 domains;
and L1 is a bond or a peptide linker;
   wherein VL and VH associate to form an Fv that specifically binds to PDL1; and
   wherein the scFv specifically binds to CD137. In some embodiments, the Fv comprises a VH comprising a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and/or a VL comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the Fv comprises a VH comprising the amino acid sequence of SEQ ID NO: 43, and/or a VL comprising the amino acid sequence of SEQ ID NO: 44. In some embodiments, the scFv comprises a VH comprising an CDR-H1 comprising the amino acid sequence of SEQ ID NO: 45, a CDR-H2 comprising the amino acid sequence of SEQ ID NO: 46, and a CDR-H3 comprising the amino acid sequence of SEQ ID NO: 47; and/or a VL comprising a CDR-L1 comprising the amino acid sequence of SEQ ID NO: 48, a CDR-L2 comprising the amino acid sequence of SEQ ID NO: 49, and a CDR-L3 comprising the amino acid sequence of SEQ ID NO: 50. In some embodiments, the scFv comprises a VH comprising the amino acid sequence of SEQ ID NO: 51 and/or a VL comprising the amino acid sequence of SEQ ID NO: 52. In some embodiments, the scFv comprises from the N-terminus to the C-terminus: VH-L1-VL, wherein L1 is a peptide linker. In some embodiment, L1 comprises the amino acid sequence of SEQ ID NO: 82. In some embodiments, the scFv comprises a first cysteine residue at position 44 in the VH and a second cysteine residue at position 100 in the VL, wherein the first cysteine residue and the second cysteine residue form a disulfide bond, and wherein the numbering is based on Kabat numbering. In some embodiments, the scFv comprises the amino acid sequence of SEQ ID NO: 78. In some embodiments, the first CH3 domain comprises N390C substitution and the second CH3 domain comprises S400C substitution, or the first CH3 domain comprises S400C substitution and the second CH3 domain comprises N390C substitution. In some embodiments, the multispecific antibody comprises an IgG1 Fc region, such as an IgG1 Fc having an N297A substitution. In some embodiments, the multispecific antibody comprises an IgG4 Fc region, such as an IgG4 having a S228P substitution. In some embodiments, the scFv is linked to the hinge in the second polypeptide via a linker, such as a peptide linker comprising the amino acid sequence of SEQ ID NO: 80 or 81.

In some embodiments, there is provided a bispecific antibody comprising a first polypeptide comprising the amino acid sequence of SEQ ID NO: 84, and a second polypeptide comprising the amino acid sequence of SEQ ID NO: 85. In some embodiments, there is provided a bispecific antibody comprising a first polypeptide comprising the amino acid sequence of SEQ ID NO: 86, and a second polypeptide comprising the amino acid sequence of SEQ ID NO: 87. In some embodiments, there is provided a bispecific antibody comprising a first polypeptide comprising the amino acid sequence of SEQ ID NO: 88, and a second polypeptide comprising the amino acid sequence of SEQ ID NO: 89. In some embodiments, there is provided a bispecific antibody comprising a first polypeptide comprising the amino acid sequence of SEQ ID NO: 90, and a second polypeptide comprising the amino acid sequence of SEQ ID NO: 91. In some embodiments, there is provided a bispecific antibody comprising a first polypeptide comprising the amino acid sequence of SEQ ID NO: 92, and a second polypeptide comprising the amino acid sequence of SEQ ID NO: 93. In some embodiments, there is provided a bispecific antibody comprising a first polypeptide comprising the amino acid sequence of SEQ ID NO: 94, and a second polypeptide comprising the amino acid sequence of SEQ ID NO: 95.

In some embodiments, there is provided a trispecific antibody targeting PDL1, CD137 and CTLA4, comprising a first polypeptide, a second polypeptide, and a third polypeptide, wherein:

(i) the first polypeptide comprises a structure represented by the formula:

VH-CH1-hinge-CH2-first CH3-L1-scFv1;

(ii) the second polypeptide comprises a structure represented by the formula:

VH-CH1-hinge-CH2-second CH3-L2-scFv2;

(iii) the third polypeptide comprises a structure represented by the formula:

VL-CL; and

(iv) the fourth polypeptide comprises a structure represented by the formula:

VL-CL;

wherein:

VH is an immunoglobulin light chain variable domain;

VL is an immunoglobulin light chain variable domain;

scFv1 is a first single-chain variable fragment;

CL is an immunoglobulin light chain constant domain;

CH1 is an immunoglobulin heavy chain constant domain 1;

CH2 is an immunoglobulin heavy chain constant domain 2:
hinge is an immunoglobulin hinge region connecting the CH1 and CH2 domains; and
L1 and L2 are independently a bond or a peptide linker;
   wherein VL and VH associate to form an Fv that specifically binds to CD137; wherein scFv1 specifically binds to PD-L1, and scFv2 specifically binds to CTLA4. In some embodiments, the scFv1 comprises a VH comprising a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and/or a VL comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the scFv1 comprises a VH comprising the amino acid sequence of SEQ ID NO: 43, and/or a VL comprising the amino acid sequence of SEQ ID NO: 44. In some embodiments, the Fv comprises a VH comprising an CDR-H1 comprising the amino acid sequence of SEQ ID NO: 45, a CDR-H2 comprising the amino acid sequence of SEQ ID NO: 46, and a CDR-H3 comprising the amino acid sequence of SEQ ID NO: 47; and/or a VL comprising a CDR-L1 comprising the amino acid sequence of SEQ ID NO: 48, a CDR-L2 comprising the amino acid sequence of SEQ ID NO: 49, and a CDR-L3 comprising the amino acid sequence of SEQ ID NO: 50. In some embodiments, the Fv comprises a VH comprising the amino acid sequence of SEQ ID NO: 51 and/or a VL comprising the amino acid sequence of SEQ ID NO: 52. In some embodiments, the scFv2 comprises a VH comprising an CDR-HI comprising the amino acid sequence of SEQ ID NO: 53, a CDR-H2 comprising the amino acid sequence of SEQ ID NO: 54, and a CDR-H3 comprising the amino acid sequence of SEQ ID NO: 55; and/or a VL comprising a CDR-L1 comprising the amino acid sequence of SEQ ID NO: 56, a CDR-L2 comprising the amino acid sequence of SEQ ID NO: 57, and a CDR-L3 comprising the amino acid sequence of SEQ ID NO: 58. In some embodiments, the scFv2 comprises a VH comprising the amino acid sequence of SEQ ID NO: 59 and/or a VL comprising the amino acid sequence of SEQ ID NO: 60. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the multispecific antibody comprises an IgG1 Fc region, such as an IgG1 Fc having an N297A substitution.

In some embodiments, there is provided a trispecific antibody comprising: a first polypeptide comprising the amino acid sequence of SEQ ID NO: 118, a second polypeptide comprising the amino acid sequence of SEQ ID NO: 119, and a third polypeptide comprising the amino acid sequence of SEQ ID NO: 120. In some embodiments, there is provided a trispecific antibody comprising: a first polypeptide comprising the amino acid sequence of SEQ ID NO: 121, a second polypeptide comprising the amino acid sequence of SEQ ID NO: 122, and a third polypeptide comprising the amino acid sequence of SEQ ID NO: 123. In some embodiments, there is provided a trispecific antibody comprising: a first polypeptide comprising the amino acid sequence of SEQ ID NO: 124, a second polypeptide comprising the amino acid sequence of SEQ ID NO: 125, and a third polypeptide comprising the amino acid sequence of SEQ ID NO: 126.

In some embodiments, there is provided a bispecific T cell engager (BiTE) molecule targeting CD3 and a tumor antigen (e.g., HER2), comprising a first polypeptide, a second polypeptide and a third polypeptide, wherein:

(i) the first polypeptide comprises a structure represented by the formula:

VH-CH1-hinge-CH2-first CH3;

(ii) the second polypeptide comprises a structure represented by the formula:

scFv-hinge-CH2-second CH3; and

(iii) the third polypeptide comprises a structure represented by the formula:

VL-CL;

wherein:

VL is an immunoglobulin light chain variable domain;

VH is an immunoglobulin heavy chain variable domain;

scFv is a single-chain variable fragment;

CL is an immunoglobulin light chain constant domain:

CH1 is an immunoglobulin heavy chain constant domain 1;

CH2 is an immunoglobulin heavy chain constant domain 2; and

hinge is an immunoglobulin hinge region connecting the CH1 and CH2 domains:

wherein VL and VH associate to form an Fv that specifically binds to the tumor antigen (e.g., HER2); and wherein the scFv specifically binds to CD3. In some embodiments, the scFv comprises a VH comprising a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the scFv comprises a VH comprising the amino acid sequence of SEQ ID NO: 67, and/or a VL comprising the amino acid sequence of SEQ ID NO: 68. In some embodiments, the scFv comprises the amino acid sequence of SEQ ID NO: 79. In some embodiments, the Fv specifically binds to HER2. In some embodiments, the Fv comprises a VH comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the Fv comprises a VH comprising the amino acid sequence of SEQ ID NO: 75 and/or a VL comprising the amino acid sequence of SEQ ID NO: 76. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the multispecific antibody comprises an IgG1 Fc region, such as an IgG1 Fc having an N297A substitution. In some embodiments, there is provided a bispecific T cell engager molecule comprising a first polypeptide comprising the amino acid sequence of SEQ ID NO: 112, a second polypeptide comprising the amino acid sequence of SEQ ID NO: 113, and a third polypeptide comprising the amino acid sequence of SEQ ID NO: 114.

### IV. ACTIVATABLE ANTIBODIES

Certain aspects of the present application relate to activatable antibodies (including activatable bispecific T cell engager molecules), activatable antigen binding fragments thereof, or derivatives of activatable antibodies.

In some embodiments, the activatable antibody comprises a polypeptide comprising a target-binding moiety (TBM), a cleavable moiety (CM), and a masking moiety (MM). In some embodiments, the TBM comprises an amino acid sequence that binds to a target such as CD3 or HER2. In some embodiments, the TBM comprises an antigen-binding domain (ABD) of an antibody or antibody fragment thereof. In some embodiments, the TBM comprises an antibody light chain variable region (VL) and an antibody heavy chain variable region (VH), wherein the VH and VL forms a binding domain that binds to the target in the absence of the MM. In some embodiments, the VH and VL are covalently linked, e.g., in a scFv. In some embodiments, the VH and VL form an Fv fragment. In some embodiments, the VH is linked to an antibody heavy chain constant region, and the VL is linked to an antibody light chain constant region. In some embodiments, the activatable antibody comprises an Fc region comprising any one or combination of the engineered disulfide bonds or salt bridges described herein. In some embodiments, the activatable antibody comprises an Fc region that does not comprise any one or combination of the engineered disulfide bonds or salt bridges described herein.

In some embodiments, the activatable antibody comprises a polypeptide comprising the structure, from N-terminus to C-terminus, of: masking moiety (MM)-cleavable moiety (CM)-VL, and the activatable antibody further comprises a second polypeptide comprising a VH (e.g., a Fab fragment). In some embodiments, the activatable antibody comprises a polypeptide comprising the structure, from N-terminus to C-terminus, of: masking moiety (MM)-cleavable moiety (CM)-VL-VH (e.g., a scFv). In some embodiments, the activatable antibody comprises a polypeptide comprising the structure, from N-terminus to C-terminus, of: masking moiety (MM)-cleavable moiety (CM)-VH, and the activatable antibody further comprises a second polypeptide comprising a VL (e.g., a Fab fragment). In some embodiments, the activatable antibody comprises a polypeptide comprising the structure, from N-terminus to C-terminus, of: masking moiety (MM)-cleavable moiety (CM)-VH-VL (e.g., a scFv).

In some embodiments, the activatable antibody comprises a polypeptide comprising the structure, from N-terminus to C-terminus, of: masking moiety (MM)-L1-cleavable moiety (CM)-L2-VL, and the activatable antibody further comprises a second polypeptide comprising a VH (e.g., a Fab fragment). In some embodiments, the activatable antibody comprises a polypeptide comprising the structure, from N-terminus to C-terminus, of: masking moiety (MM)-L1-cleavable moiety (CM)-L2-VL-L3-VH (e.g., a scFv). In some embodiments, the activatable antibody comprises a polypeptide comprising the structure, from N-terminus to C-terminus, of masking moiety (MM)-cleavable moiety (CM)-L1-VH, and the activatable antibody further comprises a second polypeptide comprising a VL (e.g., a Fab fragment). In some embodiments, the activatable antibody comprises a polypeptide comprising the structure, from N-terminus to C-terminus, of: masking moiety (MM)-L1-cleavable moiety (CM)-L2-VH-L3-VL (e.g., a scFv). In some embodiments, L1, L2, and/or L3 are linkers. In some embodiments, each of L1, L2, and L3 is a linker that can independently be either a bond or a peptide linker having an independently selected length of 1 or more, 2 or more, 3 or more, 4 or more, 5 or more, 6 or more, 7 or more, 8 or more, 9 or more, or 10 or more amino acids.

In some embodiments, there is provided an activatable antibody comprising a first polypeptide comprising a first CH3 domain, a second polypeptide comprising a second CH3 domain, and a third polypeptide, wherein:

(i) the first polypeptide comprises a structure represented by the formula:

VH-CH1-hinge-CH2-first CH3;

(ii) the second polypeptide comprises a structure represented by the formula:

MM1-CM1-scFv-hinge-CH2-second CH3; and

(iii) the third polypeptide comprises a structure represented by the formula:

MM2-CM2-VL-CL;

wherein:

VL is an immunoglobulin light chain variable domain;

VH is an immunoglobulin heavy chain variable domain;

scFv is a single-chain variable fragment:

CL is an immunoglobulin light chain constant domain;

CH1 is an immunoglobulin heavy chain constant domain 1;

CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL and VH associate to form a first Fv that specifically binds to a first target; wherein the scFv specifically binds to a second target; wherein MM1 inhibits the binding of the scFv to the first target when CM1 is not cleaved; and wherein MM2 inhibits the binding of the first Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2) and the second target is CD3 (e.g., CD3e). In some embodiments, the first target is CD3 (e.g., CD3e) and the second target is a tumor antigen (e.g., HER2).

In some embodiments, there is provided an activatable antibody comprising a first polypeptide comprising a first CH3 domain, a second polypeptide comprising a second CH3 domain, and a third polypeptide, wherein:

(i) the first polypeptide comprises a structure represented by the formula:

MM1-CM1-VH-CH1-hinge-CH2-first CH3;

(ii) the second polypeptide comprises a structure represented by the formula:

MM2-CM2-scFv-hinge-CH2-second CH3; and

(iii) the third polypeptide comprises a structure represented by the formula:

VL-CL;

wherein:

VL is an immunoglobulin light chain variable domain;

VH is an immunoglobulin heavy chain variable domain;

scFv is a single-chain variable fragment;

CL is an immunoglobulin light chain constant domain;

CH1 is an immunoglobulin heavy chain constant domain 1;

CH2 is an immunoglobulin heavy chain constant domain 2;
hinge is an immunoglobulin hinge region connecting the CH1 and CH2 domains:
MM1 is a first masking peptide;
MM2 is a second masking peptide;
CM1 is a first cleavable peptide; and
CM2 is a second cleavable peptide;
   wherein VL and VH associate to form a first Fv that specifically binds to a first target; wherein the scFv specifically binds to a second target; wherein MM1 inhibits the binding of the first Fv to the first target when CM1 is not cleaved; and wherein MM2 inhibits the binding of the scFv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D. N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2) and the second target is CD3 (e.g., CD3e). In some embodiments, the first target is CD3 (e.g., CD3e) and the second target is a tumor antigen (e.g., HER2).

In some embodiments, there is provided an activatable antibody comprising a first polypeptide comprising a first CH3 domain, a second polypeptide comprising a second CH3 domain, a third polypeptide, and a fourth polypeptide, wherein:

(i) the first polypeptide comprises a structure represented by the formula:

MM1-CM1-VH1-CH1-hinge-CH2-first CH3;

(ii) the second polypeptide comprises a structure represented by the formula:

MM2-CM2-VH2-CH1-hinge-CH2-second CH3;

(iii) the third polypeptide comprises a structure represented by the formula:

VL1-CL; and

(iv) the fourth polypeptide comprises a structure represented by the formula:

VL2-CL;

wherein:

VL1 is a first immunoglobulin light chain variable domain;

VH1 is a first immunoglobulin heavy chain variable domain;

VL2 is a second immunoglobulin light chain variable domain;

VH2 is a second immunoglobulin heavy chain variable domain;

CL is an immunoglobulin light chain constant domain;

CH1 is an immunoglobulin heavy chain constant domain 1;

CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL1 and VH1 associate to form a first Fv that specifically binds to a first target; wherein VL2 and VH2 associate to form a second Fv that specifically binds to a second target; wherein MM1 inhibits the binding of the first Fv to the first target when CM1 is not cleaved; and wherein MM2 inhibits the binding of the second Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2) and the second target is CD3 (e.g., CD3e). In some embodiments, the first target is CD3 (e.g., CD3e) and the second target is a tumor antigen (e.g., HER2).

In some embodiments, there is provided an activatable antibody comprising a first polypeptide comprising a first CH3 domain, a second polypeptide comprising a second CH3 domain, a third polypeptide, and a fourth polypeptide, wherein:

(i) the first polypeptide comprises a structure represented by the formula:

MM1-CM1-VH1-CH1-hinge-CH2-first CH3;

(ii) the second polypeptide comprises a structure represented by the formula:

VH2-CH1-hinge-CH2-second CH3;

(iii) the third polypeptide comprises a structure represented by the formula:

VL1-CL; and

(iv) the fourth polypeptide comprises a structure represented by the formula:

MM2-CM2-VL2-CL;

wherein:

VL1 is a first immunoglobulin light chain variable domain;

VH1 is a first immunoglobulin heavy chain variable domain;

VL2 is a second immunoglobulin light chain variable domain;

VH2 is a second immunoglobulin heavy chain variable domain;

CL is an immunoglobulin light chain constant domain;

CH1 is an immunoglobulin heavy chain constant domain 1;

CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL1 and VH1 associate to form a first Fv that specifically binds to a first target; wherein VL2 and VH2 associate to form a second Fv that specifically binds to a second target; wherein MM1 inhibits the binding of the first Fv to the first target when CM1 is not cleaved; and wherein MM2 inhibits the binding of the second Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D. K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2) and the second target is CD3 (e.g., CD3e). In some embodiments, the first target is CD3 (e.g., CD3e) and the second target is a tumor antigen (e.g., HER2).

In some embodiments, there is provided an activatable antibody comprising a first polypeptide comprising a first CH3 domain, a second polypeptide comprising a second CH3 domain, a third polypeptide, and a fourth polypeptide, wherein:

(i) the first polypeptide comprises a structure represented by the formula:

VH1-CH1-hinge-CH2-first CH3;

(ii) the second polypeptide comprises a structure represented by the formula:

VH2-CH1-hinge-CH2-second CH3:

(iii) the third polypeptide comprises a structure represented by the formula:

MM1-CM1-VL1-CL; and

(iv) the fourth polypeptide comprises a structure represented by the formula:

MM2-CM2-VL2-CL;

wherein:

VL1 is a first immunoglobulin light chain variable domain;

VH1 is a first immunoglobulin heavy chain variable domain;

VL2 is a second immunoglobulin light chain variable domain;

VH2 is a second immunoglobulin heavy chain variable domain;

CL is an immunoglobulin light chain constant domain;

CH1 is an immunoglobulin heavy chain constant domain 1;

CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL I and VH1 associate to form a first Fv that specifically binds to a first target; wherein VL2 and VH2 associate to form a second Fv that specifically binds to a second target; wherein MM1 inhibits the binding of the first Fv to the first target when CM1 is not cleaved; and wherein MM2 inhibits the binding of the second Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D. K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2) and the second target is CD3 (e.g., CD3e). In some embodiments, the first target is CD3 (e.g., CD3e) and the second target is a tumor antigen (e.g., HER2).

In some embodiments, the activatable antibody is designed based on any one of the multispecific antibodies described herein, e.g., by fusing a masking moiety (MM) to a target-binding moiety (TBM) in the multispecific antibody via a cleavable moiety (CM), wherein the MM inhibits binding of the TBM to its target when the CM is not cleaved. Activatable antibodies have been described, for example, in WO2019/149282, the contents of which are incorporated herein by reference in its entirety. The activatable antibody may comprise any one of the TBMs described in the subsection “Target binding moiety (TBM)” of section III “Multispecific antibodies.” The activatable antibodies described herein may comprise one or more linkers described in the subsection “Linker” of section III “Multispecific antibodies”, e.g., disposed between MM and CM, CM and TBM, or TBM and hinge region of an Fc.

The MM refers to an amino acid sequence that, when the CM of the activatable antibody is intact (e.g., uncleaved by a corresponding enzyme, and/or containing an unreduced cysteine-cysteine disulfide bond), the MM interferes with or inhibits binding of the TBM to its target. In some embodiments, the MM interferes with or inhibits binding of the TBM to its target so efficiently that binding of the TBM to its target is extremely low and/or below the limit of detection (e.g., binding cannot be detected in an ELISA or flow cytometry assay). The amino acid sequence of the CM may overlap with or be included within the MM. It should be noted that for sake of convenience “ABP” or “activatable antibody” are used herein to refer to an ABP or activatable antibody in both their uncleaved (or “native”) state, as well as in their cleaved state. It will be apparent to the ordinarily skilled artisan that in some embodiments a cleaved ABP may lack an MM due to cleavage of the CM, e.g., by a protease, resulting in release of at least the MM (e.g., where the MM is not joined to the ABP by a covalent bond (e.g., a disulfide bond between cysteine residues)). Exemplary ABPs are described in more detail below.

The CM generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a CM, the terms encompass enzymatic cleavage, e.g., by a protease, as well as disruption of a disulfide bond between a cysteine-cysteine pair via reduction of the disulfide bond that can result from exposure to a reducing agent.

In some embodiments, the activatable antibodies do not induce ADCC effects. Methods of measuring ADCC effects are known in the art. In some embodiments, the activatable antibodies (when in active form or inactive form) do not ADCC effects by more than about 10% (do not induce ADCC by more than about 10%, more than about 5%, more than about 1%, more than about 0.1%, more than about 0.01%) relative to a control.

In some embodiments, the activatable antibodies (e.g., BiTE molecules) are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the activatable antibodies and T cells relative to corresponding tumor cells not contacted with the activatable antibodies (or relative to corresponding tumor cells contacted with an isotype control antibody and T cells). In some embodiments, the activatable antibodies are capable of reducing tumor volume in a subject when the subject is administered the activatable antibodies. In some embodiments, the activatable antibodies (e.g., BiTE molecules) are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the activatable antibodies; as compared to a corresponding tumor in a subject administered an isotype control antibody). Methods of monitoring tumor cell growth/proliferation, tumor volume, and/or tumor inhibition are known in the art.

In some embodiments, the activatable antibodies have therapeutic effect on a cancer. In some embodiments, the activatable antibodies reduce one or more signs or symptoms of a cancer. In some embodiments, a subject suffering from a cancer goes into partial or complete remission when administered the activatable antibodies.

### Masking Moiety (MM)

The activatable antibodies described herein comprise one, two or more masking moieties. Sequences of exemplary masking moieties are shown in Table D below. Masking moieties can be isolated from phage display libraries, for example, as described in WO2019/149282, which is incorporated herein by reference in its entirety.

In some embodiments, the activatable antibody comprises a MM comprising the amino acid sequence of SEQ ID NO: 35. In some embodiments, the activatable antibody comprises a MM comprising the amino acid sequence of SEQ ID NO: 36. In some embodiments, the activatable antibody comprises a first MM comprising the amino acid sequence of SEQ ID NO: 35, and a second MM comprising the amino acid sequence of SEQ ID NO: 36.

TABLE D
Masking Moieties.

| | Masking | Sequence |
|---|---|---|
| SEQ ID NQ | moiety | |
| 35 | CD3 MM | EVGSYPYDDPDCPSHDSDCDN |
| 36 | HER2 MM | ESDACDADPFDCQAGGGGSGSGGS |

In some embodiments, the masking peptide (MM) interferes with, obstructs, reduces the ability of, prevents, inhibits, or competes with the corresponding target binding moiety for binding to its target (e.g., an “inactive activatable antibody). In some embodiments, the masking peptide (MM) interferes with, obstructs, reduces, prevents, inhibits, or competes with the target binding moiety for binding to its target only when the antibody has not been activated (e.g., activated by a change in pH (increased or decreased), activated by a temperature shift (increased or decreased), activated after being contacted with a second molecule (such as a small molecule or a protein ligand), etc.). In some embodiments, activation induces cleavage of the cleavable moiety. In some embodiments, activation induces conformation changes in the polypeptide(s) (e.g., displacement of the MM), leading to the MM no longer preventing the activatable antibody from binding to its target. In some embodiments, the MM interferes with, obstructs, reduces the ability of, prevents, inhibits, or competes with the target binding moiety for binding to its target only when the cleavable moiety (CM) has not been cleaved by one or more proteases that cleave within the cleavable moiety (CM). In some embodiments, the MM has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) prior to activation. In some embodiments, masking efficiency is measured as the difference in affinity of an activatable antibody comprising the MM for binding its target (before activation) relative to the affinity of a polypeptide lacking the MM for binding its target (e.g., the difference in affinity for a target antigen (such as CD3 or HER2) of an activatable antibody comprising a MM (before activation) relative to a parental antibody lacking the MM, or the difference in affinity for a target antigen (such as CD3 or HER2) of an activatable antibody comprising a MM (before activation) relative to the affinity for the target antigen of the activatable antibody after activation). In some embodiments, the masking efficiency is measured by dividing the EC50 for binding of an activatable antibody comprising a MM (before activation) by the EC50 of the parental antibody (e.g., by measuring EC50 by ELISA). In some embodiments, masking efficiency is measured as the difference in affinity of an activatable antibody comprising the MM for binding its target before activation relative to the affinity of the activatable antibody comprising the MM for binding its target after activation (e.g., the difference in affinity for a target antigen (such as CD3 or HER2) of an activatable antibody before activation relative to the activatable antibody after activation). In some embodiments, the MM binds to the target binding moiety (TBM), and prevents the activatable antibody from binding to its target (e.g., an “inactive” activatable antibody). In some embodiments, the MM has a dissociation constant for binding to the target binding moiety (TBM) that is greater than the dissociation constant of the target binding moiety (TBM) for its target. Dissociation constants can be measured, e.g., by techniques such as ELISA, surface plasmon resonance or Bio-Layer Interferometry (BLI), or flow cytometry.

In some embodiments, the MM does not interfere with, obstruct, reduce the ability of, prevent, inhibit, or compete with the target binding moiety (TBM) for binding to its target after the polypeptide has been activated (e.g., activated by treatment with one or more proteases that cleave within the cleavable moiety (CM), activated by a change in pH (increased or decreased), activated by a temperature shift (increased or decreased), activated after being contacted with a second molecule (such as an enzyme), etc.). In some embodiments, the MM does not interfere with, obstruct, reduce the ability of, prevent, inhibit, or compete with the target binding moiety (TBM) for binding to its target after the cleavable moiety (CM) has been cleaved by one or more proteases that cleave within the cleavable moiety (CM). In some embodiments, the MM has a masking efficiency of at most about 1.75 (e.g., at most about 1.75, at most about 1.5, at most about 1.4, at most about 1.3, at most about 1.2, at most about 1.1, at most about 1.0, at most about 0.9, at most about 0.8, at most about 0.7, at most about 0.6, or at most about 0.5, etc.) after activation (e.g., the relative affinity of the activatable antibody after activation as compared to the affinity of a parental antibody).

In some embodiments, any of the MMs described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the MM.

### Cleavable Moiety (CM)

In some embodiments, the activatable antibody comprises one or more CMs, each of which is disposed between a MM and a TBM.

In some embodiments, the CM comprises at least a first cleavage site (CS1) (e.g., a first protease cleavage site). In some embodiments, the first cleavage site is a first protease cleavage site. Any suitable protease cleavage site recognized and/or cleaved by any protease (e.g., a protease that is known to be co-localized with a target of a polypeptide comprising the CM) known in the art may be used, including, for example, a protease cleavage site recognized and/or cleaved by urokinase-type plasminogen activator (uPA): matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM 8, ADAM 9, ADAMIO, ADAM12, ADAMIS, ADAM17/TACE, ADAMDECI, ADAMTSI, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14): aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A, Cathepsin G, Chymase, and/or coagulation factor proteases (such as FVIIa, FIXa, FXa, FXla, FXIIa)); elastase: granzyme B; guanidinobenzoatase; HtrAl; human neutrophil elastase; lactoferrin; marapsin; NS3/4A; PACE4; tPA; tryptase: type 11 transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc. In some embodiments, the first protease cleavage site is a cleavage site for a protease selected from uPA, MMP-1, MMP-2, MMP-3, MMP-8, MMP-9, MMP-14, TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first protease cleavage site is a cleavage site for a protease selected from uPA, MMP-2, MMP-9, and/or TEV protease. In some embodiments, the protease cleavage comprises an amino acid sequence selected from SGRSA (SEQ ID NO: 127), PLGLAG (SEQ ID NO: 128), and ENLYFQG (SEQ ID NO: 129).

In some embodiments, the cleavable moiety (CM) further comprises at least a second cleavage site (e.g., at least a second, at least a third, at least a fourth, at least a fifth, etc.). In some embodiments, the cleavable moiety (CM) further comprises a second cleavage site (CS2). In some embodiments, the second cleavage site is a second protease cleavage site. The second protease cleavage site may be any suitable protease cleavage site recognized and/or cleaved by any of the proteases described above. In some embodiments, the first (CS1) and second (CS2) cleavage sites are protease cleavage sites recognized and/or cleaved by the same protease. In some embodiments, the first (CS1) and second (CS2) cleavage sites are protease cleavage sites recognized and/or cleaved by different proteases (e.g., the first protease cleavage site is recognized and/or cleaved by uPA, and the second protease cleavage site is recognized and/or cleaved by MMP-2; the first protease cleavage site is recognized and/or cleaved by uPA, and the second protease cleavage site is recognized and/or cleaved by MMP-9; the first protease cleavage site is recognized and/or cleaved by uPA, and the second protease cleavage site is recognized and/or cleaved by TEV protease: etc.). In some embodiments, the at least second cleavage site (CS2) is C-terminal to the first linker (L1). In some embodiments, the cleavable moiety (CM) comprises a structure, from N-terminus to C-terminus, of; (CS1)-L1-(CS2).

In some embodiments, the cleavable moiety (CM) further comprises at least a second linker (e.g., at least a second, at least a third, at least a fourth, at least a fifth, etc.). In some embodiments, the cleavable moiety (CM) further comprises a second linker (L2). The second linker (L2) may be any suitable linker described above. In some embodiments, the first (L1) and second (L2) linkers are the same. In some embodiments, the first (L1) and second (L2) linkers are different. In some embodiments, the at least second linker (L2) is C-terminal to the second cleavage site (CS2). In some embodiments, the cleavable moiety (CM) comprises a structure, from N-terminus to C-terminus, of (CS1)-L1-(CS2)-L2.

### Activatable Antibodies Targeting CD3

The present application provides activatable antibodies, activatable antibody fragments, and polypeptides that target CD3, comprising a masking moiety (MM) comprising the amino acid sequence of SEQ ID NO: 35.

In some embodiments, there is provided an antibody light chain comprising a polypeptide comprising, from N-terminus to C-terminus, a MM, a cleavable moiety (CM), and a target binding moiety (TBM), wherein the MM comprises the amino acid sequence of SEQ ID NO: 35; wherein the CM comprises at least a first cleavage site; and wherein the TBM comprises a VL of an anti-CD3 antibody. In some embodiments, the anti-CD3 antibody comprises a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66.

In some embodiments, there is provided an antibody heavy chain comprising a polypeptide comprising, from N-terminus to C-terminus, a MM, a CM, and a TBM, wherein the MM comprises the amino acid sequence of SEQ ID NO: 35; wherein the CM comprises at least a first cleavage site; and wherein the TBM comprises a VH of an anti-CD3 antibody. In some embodiments, the anti-CD3 antibody comprises a VH comprising a CDR-H1 comprising the amino acid sequence of SEQ ID NO: 61, a CDR-H2 comprising the amino acid sequence of SEQ ID NO: 62.

In some embodiments, there is provided an activatable antibody targeting CD3 comprising a first polypeptide comprising, from N-terminus to C-terminus, a MM, a CM, and a TBM, wherein the MM comprises the amino acid sequence of SEQ ID NO: 35, wherein the MM inhibits binding of the activatable antibody to CD3 when the CM is not cleaved; wherein the CM comprises at least a first cleavage site; wherein the TBM comprises a VL, and the activatable antibody further comprises a second polypeptide comprising a VH; and wherein the activatable antibody binds to CD3 via the VH and VL when the CM is cleaved. In some embodiments, the activatable antibody comprises a VH comprises a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the activatable antibody comprises a VH comprising the amino acid sequence of SEQ ID NO: 67, and/or a VL comprising the amino acid sequence of SEQ ID NO: 68.

In some embodiments, there is provided an activatable antibody targeting CD3 comprising a first polypeptide comprising, from N-terminus to C-terminus, a MM, a CM, and a TBM, wherein the MM comprises the amino acid sequence of SEQ ID NO: 35, wherein the MM inhibits binding of the activatable antibody to CD3 when the CM is not cleaved; wherein the CM comprises at least a first cleavage site; wherein the TBM comprises a VH, and the activatable antibody further comprises a second polypeptide comprising a VL; and wherein the activatable antibody binds to CD3 via the VH and VL when the CM is cleaved. In some embodiments, the activatable antibody comprises a VH comprises a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the activatable antibody comprises a VH comprising the amino acid sequence of SEQ ID NO: 67, and/or a VL comprising the amino acid sequence of SEQ ID NO: 68.

In some embodiments, there is provided an activatable antibody targeting CD3 comprising a first polypeptide comprising, from N-terminus to C-terminus, a MM, a CM, and a scFv, wherein the MM comprises the amino acid sequence of SEQ ID NO: 35, wherein the MM inhibits binding of the activatable antibody to CD3 when the CM is not cleaved; wherein the CM comprises at least a first cleavage site; and wherein the activatable antibody binds to CD3 via the scFv when the CM is cleaved. In some embodiments, the scFv comprises a VH comprises a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the scFv comprises a VH comprising the amino acid sequence of SEQ ID NO: 67, and/or a VL comprising the amino acid sequence of SEQ ID NO: 68. In some embodiments, the scFv comprises from the N-terminus to the C-terminus: a VL and a VH. In some embodiments, the scFv comprises from the N-terminus to the C-terminus: a VH and a VL. In some embodiments, the scFv comprises the amino acid sequence of SEQ ID NO: 79.

In some embodiments, the activatable antibody targeting CD3 is a multispecific antibody, such as a bispecific antibody. In some embodiments, the activatable antibody targeting CD3 is a bispecific T cell engager (BiTE) molecule, which also targets a tumor antigen, such as HER2.

In some embodiments, there is provided an activatable bispecific T cell engager molecule, comprising: a first polypeptide comprising a first CH3 domain, a second polypeptide comprising a second CH3 domain, and a third polypeptide, wherein:

(i) the first polypeptide comprises a structure represented by the formula:

VH-CH1-hinge-CH2-first CH3;

(ii) the second polypeptide comprises a structure represented by the formula:

MM1-CM1-scFv-hinge-CH2-second CH3; and

(iii) the third polypeptide comprises a structure represented by the formula:

MM2-CM2-VL-CL;

wherein:

VL is an immunoglobulin light chain variable domain;

VH is an immunoglobulin heavy chain variable domain;

scFv is a single-chain variable fragment;

CL is an immunoglobulin light chain constant domain;

CH1 is an immunoglobulin heavy chain constant domain 1;

CH2 is an immunoglobulin heavy chain constant domain 2;
hinge is an immunoglobulin hinge region connecting the CH1 and CH2 domains;
MM1 is a first masking peptide;
MM2 is a second masking peptide:
CM1 is a first cleavable peptide; and
CM2 is a second cleavable peptide:
   wherein VL and VH associate to form a first Fv that specifically binds to a tumor antigen (e.g., HER2); wherein the scFv specifically binds to CD3; wherein MM1 inhibits the binding of the scFv to CD3 when CM1 is not cleaved; and wherein MM2 inhibits the binding of the first Fv to the tumor antigen (e.g., HER2) when CM2 is not cleaved. In some embodiments, the MM1 comprises the amino acid sequence of SEQ ID NO: 35. In some embodiments, the scFv comprises a VH comprises a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the scFv comprises a VH comprising the amino acid sequence of SEQ ID NO: 67, and/or a VL comprising the amino acid sequence of SEQ ID NO: 68. In some embodiments, the scFv comprises the amino acid sequence of SEQ ID NO: 79.

In some embodiments, the activatable BiTE molecule targets HER2. In some embodiments, the MM2 comprises the amino acid sequence of SEQ ID NO: 36. In some embodiments, the VH comprises an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71. In some embodiments, the VL comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH comprises the amino acid sequence of SEQ ID NO: 75. In some embodiments, the VL comprises the amino acid sequence of SEQ ID NO: 76.

In some embodiments according to any one of the activatable antibodies (including BITE molecules) targeting CD3 described herein, the activatable antibody comprises a first CH3 domain and a second CH3 domain that do not comprise any one or combination of the engineered disulfide bonds or salt bridges described herein. In some embodiments, the activatable antibody comprises a first CH3 domain and a second CH3 domain that comprise any one or combination of the engineered disulfide bonds or salt bridges described herein. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution.

Exemplary BiTE molecules are shown, for example, in Tables 10 and 11. In some embodiments, there is provided an activatable bispecific T cell engager molecule comprising a first polypeptide comprising the amino acid sequence of SEQ ID NO: 115, a second polypeptide comprising the amino acid sequence of SEQ ID NO: 116, and a third polypeptide comprising the amino acid sequence of SEQ ID NO: 117.

### Activatable Antibodies Targeting HER2

The present application provides activatable antibodies, activatable antibody fragments, and polypeptides that target HER2, comprising a masking moiety (MM) comprising the amino acid sequence of SEQ ID NO: 36.

In some embodiments, there is provided an antibody light chain comprising a polypeptide comprising, from N-terminus to C-terminus, a MM, a cleavable moiety (CM), and a target binding moiety (TBM), wherein the MM comprises the amino acid sequence of SEQ ID NO: 36; wherein the CM comprises at least a first cleavage site; and wherein the TBM comprises a VL of an anti-HER2 antibody. In some embodiments, the anti-HER2 antibody comprises a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74.

In some embodiments, there is provided an antibody heavy chain comprising a polypeptide comprising, from N-terminus to C-terminus, a MM, a CM, and a TBM, wherein the MM comprises the amino acid sequence of SEQ ID NO: 36; wherein the CM comprises at least a first cleavage site; and wherein the TBM comprises a VH of an anti-HER2 antibody. In some embodiments, the anti-HER2 antibody comprises a VH comprising an CDR-HI comprising the amino acid sequence of SEQ ID NO: 69, a CDR-H2 comprising the amino acid sequence of SEQ ID NO: 70, and a CDR-H3 comprising the amino acid sequence of SEQ ID NO: 71.

In some embodiments, there is provided an activatable antibody targeting HER2 comprising a first polypeptide comprising, from N-terminus to C-terminus, a MM, a CM, and a TBM, wherein the MM comprises the amino acid sequence of SEQ ID NO: 36, wherein the MM inhibits binding of the activatable antibody to HER2 when the CM is not cleaved; wherein the CM comprises at least a first cleavage site; wherein the TBM comprises a VL, and the activatable antibody further comprises a second polypeptide comprising a VH; and wherein the activatable antibody binds to HER2 via the VH and VL when the CM is cleaved. In some embodiments, the activatable antibody comprises a VH comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the activatable antibody comprises a VH comprising the amino acid sequence of SEQ ID NO: 75, and/or a VL comprising the amino acid sequence of SEQ ID NO: 76.

In some embodiments, there is provided an activatable antibody targeting HER2 comprising a first polypeptide comprising, from N-terminus to C-terminus, a MM, a CM, and a TBM, wherein the MM comprises the amino acid sequence of SEQ ID NO: 36, wherein the MM inhibits binding of the activatable antibody to HER2 when the CM is not cleaved; wherein the CM comprises at least a first cleavage site; wherein the TBM comprises a VH, and the activatable antibody further comprises a second polypeptide comprising a VL; and wherein the activatable antibody binds to HER2 via the VH and VL when the CM is cleaved. In some embodiments, the activatable antibody comprises a VH comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the activatable antibody comprises a VH comprising the amino acid sequence of SEQ ID NO: 75, and/or a VL comprising the amino acid sequence of SEQ ID NO: 76.

In some embodiments, there is provided an activatable antibody targeting HER2 comprising a first polypeptide comprising, from N-terminus to C-terminus, a MM, a CM, and a scFv, wherein the MM comprises the amino acid sequence of SEQ ID NO: 36, wherein the MM inhibits binding of the activatable antibody to HER2 when the CM is not cleaved; wherein the CM comprises at least a first cleavage site; and wherein the activatable antibody binds to HER2 via the scFv when the CM is cleaved. In some embodiments, the scFv comprises a VH comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the scFv comprises a VH comprising the amino acid sequence of SEQ ID NO: 75, and/or a VL comprising the amino acid sequence of SEQ ID NO: 76. In some embodiments, the scFv comprises from the N-terminus to the C-terminus: a VL and a VH. In some embodiments, the scFv comprises from the N-terminus to the C-terminus: a VH and a VL. In some embodiments, the scFv comprises the amino acid sequence of SEQ ID NO: 79.

In some embodiments, the activatable antibody targeting HER2 is a multispecific antibody, such as a bispecific antibody. In some embodiments, the activatable antibody targeting HER2 is a bispecific T cell engager (BiTE) molecule, which also targets CD3.

In some embodiments according to any one of the activatable antibodies (including BiTE molecules) targeting HER2 described herein, the activatable antibody comprises a first CH3 domain and a second CH3 domain that do not comprise any one or combination of the engineered disulfide bonds or salt bridges described herein. In some embodiments, the activatable antibody comprises a first CH3 domain and a second CH3 domain that comprise any one or combination of the engineered disulfide bonds or salt bridges described herein. In some embodiments, the first CH3 domain comprises D356K, E357K, S364K and S400C substitutions and the second CH3 domain comprises L351D, K370D, N390C and K439D substitutions, or the first CH3 domain comprises L351D, K370D. N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution.

### V. VARIANTS AND DERIVATIVES

Also contemplated herein are variants and derivatives of any one of the heterodimeric proteins, multispecific antibodies, and activatable antibodies described herein.

In some embodiments, the heterodimeric protein or antibody derivative is derived from modifications of the amino acid sequences of the parent heterodimeric protein or antibody while conserving the overall molecular structure of the parent heterodimeric protein or antibody. Amino acid sequences of any regions of the parent heterodimeric protein or antibody chains may be modified, such as framework regions, CDR regions, or constant regions. Types of modifications include substitutions, insertions, deletions, or combinations thereof, of one or more amino acids of the parent heterodimeric protein or antibody.

In some embodiments, the antibody (e.g., multispecific antibody or activatable antibody) derivative comprises a polypeptide that is at least 80%, at least 85%, at least 90%, at least 91%, at least 92%, at least 93%, at least 94%, at least 95%, at least 96%, at least 97%, at least 98%, or at least 99% identical to the amino acid sequence as set forth in any of SEQ ID NOs: 84-143. In some embodiments, the antibody (e.g., multispecific antibody or activatable antibody) derivative comprises a VL or VH region that is at least 80%, at least 85%, at least 90%, at least 91%, at least 92%, at least 93%, at least 94%, at least 95%, at least 96%, at least 97%, at least 98%, or at least 99% identical to the amino acid sequence as set forth in any of SEQ ID NOs: 43, 44, 51, 52, 59, 60, 67, 68, 75 and 76. In some embodiments, the antibody derivative comprises a CDR-HI amino acid sequence region that is at least 80%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOs: 37, 45, 53, 61, and 69. In some embodiments, the antibody derivative comprises a CDR-H2 amino acid sequence region that is at least 80%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOs: 38, 46, 54, 62, and 70. In some embodiments, the antibody derivative comprises a CDR-H3 amino acid sequence region that is at least 80%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOs: 39, 47, 55, 63, and 71. In some embodiments, the antibody derivative comprises a CDR-L1 amino acid sequence region that is at least 80%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOs: 40, 48, 56, 64, and 72. In some embodiments, the antibody derivative comprises a CDR-L2 amino acid sequence region that is at least 80%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOs: 41, 49, 57, 65, and 73. In some embodiments, the antibody derivative comprises a CDR-L3 amino acid sequence region that is at least 80%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOs: 42, 50, 58, 66, and 74.

In some embodiments, the heterodimeric protein or antibody derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence of a heterodimeric protein or antibody described herein.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of an antibody, including the CDRs, framework regions, or constant regions. In some embodiments, the present application provides an antibody derivative that contains the VH and VL CDR sequences of an illustrative antibody described herein, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kabat et al., Sequences of Proteins of Immunological Interest. Fifth Edition, U.S. Department of Health and Human Services, NIH Publication No. 91-3242 (1991): Tomlinson et al., J. Mal. Biol. 227:776-798 (1992); and Cox et al., Eur. J. Immunol. 24:827-836 (1994)). Framework sequences that may be used in constructing an antibody derivative include those that are structurally similar to the framework sequences used by illustrative antibodies of the application For example, the CDR-H1, CDR-H2, and CDR-H3 sequences, and the CDR-L1, CDR-L2, and CDR-L3 sequences of an illustrative antibody can be grafted onto framework regions that have the identical sequence as that found in the germline immunoglobulin gene from which the framework sequence derive, or the CDR sequences can be grafted onto framework regions that contain one or more mutations as compared to the germline sequences.

In some embodiments, the antibody derivative is a chimeric antibody, which comprises an amino acid sequence of an illustrative antibody described herein. In one example, one or more CDRs from one or more illustrative antibodies are combined with CDRs from an antibody from a non-human animal, such as mouse or rat. In another example, all of the CDRs of the chimeric antibody are derived from one or more illustrative antibodies. In some particular embodiments, the chimeric antibody comprises one, two, or three CDRs from the heavy chain variable region and/or one, two, or three CDRs from the light chain variable region of an illustrative antibody. Chimeric antibodies can be generated using conventional methods known in the art.

Another type of modification is to mutate amino acid residues within the CDR regions of the VH and/or VL chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within a CDR region are altered. In some embodiments, the antibody derivative comprises 1, 2, 3, or 4 amino acid substitutions in the heavy chain CDRs and/or in the light chain CDRs. In another embodiment, the amino acid substitution is to change one or more cysteines in an antibody to another residue, such as, without limitation, alanine or serine. The cysteine may be a canonical or non-canonical cysteine. In some embodiments, the antibody derivative has 1, 2, 3, or 4 conservative amino acid substitutions in the heavy chain CDR regions relative to the amino acid sequences of an illustrative antibody.

Modifications may also be made to the framework residues within the VH and/or VL regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

In some embodiments, the Fc region of the heterodimeric protein or antibody described herein has at least one (e.g., at least one, two or three or more) amino acid substitution in addition to the amino acid substitutions that form engineered disulfide bonds or salt bridges as described herein compared to the Fc region of a wild-type IgG or a wild-type antibody. In some embodiments, the Fc region has at least 80%, at least 85%, at least 90%, at least 95% or more homology with a native sequence Fc region and/or with an Fc region of a parent polypeptide.

Furthermore, the Fc region may be modified to alter its potential glycosylation site or pattern in accordance with routine experimentation known in the art. In another aspect, the present application provides a derivative of a heterodimeric protein or antibody described herein that contains at least one mutation in a variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In some embodiments, the present application provides a derivative of an antibody described herein having a potential N-linked glycosylation site at asparagine in the heavy chain variable region, wherein the potential N-linked glycosylation site in one heavy chain variable region is removed. In some embodiments, the present application provides a derivative of an antibody described herein having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the application of which incorporated herein by reference.

In some embodiments, the antibodies described herein (e.g., multispecific antibodies and activatable antibodies) may be in any class, such as IgG, IgM, IgE, IgA, or IgD. In some embodiments, the activatable antibodies described herein (e.g., a CD3 and/or HER2 antibody) are in the IgG class, such as IgG1, IgG2, IgG3, or IgG4 subclass. An antibody described herein antibody can be converted from one class or subclass to another class or subclass using methods known in the art. An exemplary method for producing an antibody in a desired class or subclass comprises the steps of isolating a nucleic acid encoding a heavy chain of an antibody described herein (e.g., multispecific or activatable antibody) and a nucleic acid encoding a light chain of an antibody described herein (e.g., multispecific or activatable antibody), isolating the sequence encoding the VH region, ligating the VH sequence to a sequence encoding a heavy chain constant region of the desired class or subclass, expressing the light chain gene and the heavy chain construct in a cell, and collecting the antibody.

Heterodimeric proteins or antibody variants are also provided with amino-terminal leader extensions. For example, one or more amino acid residues of the amino-terminal leader sequence are present at the amino-terminus of any one or more heavy or light chains of an antibody.

The heterodimeric proteins or antibodies (e.g., multispecific antibodies or activatable antibodies) described herein may be further modified. In some embodiments, the heterodimeric protein or antibody is linked to an additional molecular entity. Examples of additional molecular entities include pharmaceutical agents, peptides or proteins, detection agent or labels, and antibodies.

In some embodiments, a heterodimeric protein or antibody of the present application is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and purompycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine131, indiumrn, yttrium90 and lutetium 177. Methods for linking a polypeptide to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug Deliv. Rev. 55:199-215 (2003); Trail, et al., Cancer Immunol. Immunother. 52:328-337 (2003); Payne, Cancer Cell 3:207-212 (2003): Allen, Nat. Rev. Cancer 2:750-763 (2002); Pastan and Kreitman, Curr. Opin. Investig. Drugs 3: 1089-1091 (2002); Senter and Springer (2001) Adv. Drug De/iv. Rev. 53:247-264.

In some embodiments, a heterodimeric protein or antibody of the present application is conjugated to a label and/or a cytotoxic agent. As used herein, a label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

As used herein, a cytotoxic agent is a moiety that reduces the proliferative capacity of one or more cells. A cell has reduced proliferative capacity when the cell becomes less able to proliferate, for example, because the cell undergoes apoptosis or otherwise dies, the cell fails to proceed through the cell cycle and/or fails to divide, the cell differentiates, etc. Nonlimiting exemplary cytotoxic agents include, but are not limited to, radioisotopes, toxins, and chemotherapeutic agents. One skilled in the art can select a suitable cytotoxic according to the intended application.

In some embodiments, a label and/or a cytotoxic agent is conjugated to a heterodimeric protein or antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label and/or cytotoxic agent is a polypeptide, the label and/or cytotoxic agent can be expressed from the same expression vector with at least one antibody chain to produce a polypeptide comprising the label and/or cytotoxic agent fused to an antibody chain. One skilled in the art can select a suitable method for conjugating a label and/or cytotoxic agent to an antibody according to the intended application.

### VI. METHODS OF PREPARATION

In one aspect, the present application provides methods for preparing the heterodimeric proteins, multispecific antibodies, or activatable antibodies described herein. For example, methods for preparing a heterodimeric protein (e.g., multispecific antibody) or activatable antibody comprising culturing a host cell comprising one or more nucleic acid(s) or vector(s) that encode heterodimeric protein (e.g., multispecific antibody) or activatable antibody polypeptides under conditions that allow expression of the nucleic acid(s) or vector, and recovering the heterodimeric protein polypeptides or activatable antibody polypeptides from the host cell culture are provided.

Polypeptides (e.g., any of the heterodimeric proteins, multispecific antibodies or activatable antibodies described above) of the present application may be produced using recombinant methods and compositions, e.g., as described in U.S. Pat. No. 4,816,567. In some embodiments, isolated nucleic acids encoding any or the polypeptides (e.g., any of the heterodimeric proteins, multispecific antibodies or activatable antibodies described above) are provided. In some embodiments, there is provided one or more nucleic acids encoding the first polypeptide and/or the second polypeptide of a heterodimeric protein. In some embodiments, there is provided one or more nucleic acids encoding an amino acid sequence comprising the VL(s) and/or an amino acid sequence comprising the VH(s) of the multispecific antibodies or activatable antibodies (e.g., the light and/or heavy chains of the antibodies). In some embodiments, one or more vectors (e.g., expression vectors) comprising such nucleic acids are provided herein. In some embodiments, a host cell comprising (e.g., has been transformed with) one or more vectors comprising nucleic acid(s) encoding the heterodimeric protein, multispecific antibody, or activatable antibody described herein. In some embodiments, the host cell is eukaryotic, e.g. a yeast cell, an insect cell, a Chinese Hamster Ovary (CHO) cell or lymphoid cell (e.g., YO, NS0, Sp20 cell).

For recombinant production of polypeptides (e.g., any of the heterodimeric proteins, multispecific antibodies or activatable antibodies described above) of the present application, nucleic acid encoding a polypeptide (e.g., a heterodimeric protein, multispecific antibody or activatable antibody described above), e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the polypeptide(s)).

Suitable host cells for cloning or expression of poly peptide-encoding vectors include prokaryotic or eukaryotic cells. For example, polypeptides may be produced in bacteria, in particular when glycosylation and Fc effector function are not needed (see. e.g., U.S. Pat. Nos. 5,648,237, 5,789,199, and 5,840,523; See also Charlton, Methods in Molecular Biology. Vol. 248 (B. K. C. Lo, ed., Humana Press, Totowa, N.J., 2003), pp. 245-254, describing expression of antibody fragments in E. coli). After expression, the polypeptide may be isolated from the bacterial cell paste in a soluble fraction and may be further purified.

In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a polypeptide with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7): human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK: buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see. e.g., Yazaki and Wu, Methods in Molecular Biology, Vol. 248 (B. K. C. Lo, ed., Humana Press, Totowa, N.J.), pp. 255-268 (2003).

In order for some secreted proteins to express and secrete in large quantities, a leader sequence from a heterologous protein may be desirable. In some embodiments, employing heterologous leader sequences may be advantageous in that a resulting mature polypeptide may remain unaltered as the leader sequence is removed in the ER during the secretion process. The addition of a heterologous leader sequence may be required to express and secrete some proteins.

Certain exemplary leader sequence sequences are described, e.g., in the online Leader sequence Database maintained by the Department of Biochemistry, National University of Singapore. See Choo et al., BMC Bioinformatics. 6: 249 (2005); and PCT Publication No. WO 2006/081430.

### VII. COMPOSITIONS AND KITS

In some embodiments, the present application provides pharmaceutical compositions comprising any one of the heterodimeric proteins, multispecific antibodies, or activatable antibodies disclosed herein, and a pharmaceutically acceptable carrier. The compositions can be prepared by conventional methods known in the art.

The term “pharmaceutically acceptable carrier” refers to any inactive substance that is suitable for use in a formulation for the delivery of a polypeptide (e.g., a heterodimeric protein, multispecific antibody, or activatable antibody). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering a polypeptide (e.g., a heterodimeric protein, multispecific antibody, or activatable antibody) is a sterile liquid, such as a solution, suspension, or dispersion, for injection or infusion. Sterile solutions can be prepared by incorporating the polypeptide (e.g., a heterodimeric protein, multispecific antibody, or activatable antibody) in the required amount in an appropriate carrier, followed by sterilization microfiltration. Dispersions may be prepared by incorporating the polypeptide into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of a polypeptide (e.g., a heterodimeric protein, multispecific antibody, or activatable antibody) included in the composition will vary depending upon a number of factors, such as the specific polypeptide and carriers used, dosage form, and desired release and pharmacodynamic characteristics. The amount of a (e.g., a heterodimeric protein, multispecific antibody, or activatable antibody) in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the polypeptide (e.g., a heterodimeric protein, multispecific antibody, or activatable antibody), one or more additional therapeutic agents may be included in the composition. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent, which produces a therapeutic effect, but may be a lesser amount as well.

Any of the polypeptides (e.g., a heterodimeric protein, multispecific antibody, or activatable antibody) and/or compositions (e.g., pharmaceutical compositions) described herein may be used in the preparation of a medicament (e.g., a medicament for use in treating or delaying progression of cancer in a subject in need thereof).

In some embodiments, provided herein is a kit comprising any one of the heterodimeric proteins, multispecific antibodies, activatable antibodies and/or compositions described herein. In some embodiments, the kit further comprises a package insert comprising instructions for use of the heterodimeric proteins, multispecific antibodies, activatable antibodies and/or compositions. The package insert may contain information about the indications, usage, dosage, administration, combination therapy, contraindications and/or warnings concerning the use of a therapeutic product. In some embodiments, the kit further comprises one or more buffers, e.g., for storing, transferring, administering, or otherwise using the heterodimeric proteins, multispecific antibodies, activatable antibodies and/or compositions. In some embodiments, the kit further comprises one or more containers for storing or administering (e.g., syringes, etc.) the heterodimeric proteins, multispecific antibodies, activatable antibodies and/or compositions. Also provided are articles of manufacture comprising any one of the heterodimeric proteins, multispecific antibodies, activatable antibodies and/or compositions described herein.

### VII. METHODS OF USE

The heterodimeric proteins, multispecific antibodies, activatable antibodies, and pharmaceutical compositions described herein are useful for therapeutic, diagnostic, or other purposes, such as modulating an immune response, treating cancer, enhancing efficacy of other cancer therapy, enhancing vaccine efficacy, or treating autoimmune diseases.

In some embodiments, there is provided a method for treating a disease or condition in a subject in need thereof comprising administering to the subject an effective amount of a pharmaceutical composition comprising any one of the heterodimeric proteins, multispecific antibodies, or activatable antibodies (e.g., activatable BiTE molecules) described herein. In some embodiments, the disease or condition is cancer. A variety of cancers may be treated or prevented with a method, use, or pharmaceutical composition provided by the present application.

In some embodiments, there is provided a method for treating a cancer in a subject in need thereof comprising administering to the subject an effective amount of a pharmaceutical composition comprising any one of the multispecific antibodies targeting one or more immune checkpoint molecules (e.g., any one of the PDL1×CD137, CD137×PDL1, or PDL1×CD137×CTLA4 antibodies) described herein. In some embodiments, the cancer is lung cancer. In some embodiments, the cancer is prostate cancer. In some embodiments, the cancer is melanoma. In some embodiments, the cancer is an advanced-stage cancer.

In some embodiments, there is provided a method for treating a cancer in a subject in need thereof comprising administering to the subject an effective amount of a pharmaceutical composition comprising any one of the BiTE or activatable BiTE molecules (e.g., any one of the HER2×CD3 antibodies or activatable HER2×CD3 antibodies) described herein. In some embodiments, the cancer is HER2-positive cancer. In some embodiments, the cancer is ovarian cancer.

In some embodiments, there is provided a method of enhancing an immune response in a mammal, which comprises administering to the mammal an effective amount of a pharmaceutical composition comprising any one of the heterodimeric proteins, multispecific antibodies, or activatable antibodies (e.g., activatable BiTE molecules) described herein. The term “enhancing immune response” or its grammatical variations, means stimulating, evoking, increasing, improving, or augmenting any response of a subject's immune system. The immune response may be a cellular response (i.e. cell-mediated, such as cytotoxic T lymphocyte mediated) or a humoral response (i.e. antibody-mediated response), and may be a primary or secondary immune response. Examples of enhancement of immune response include activation of PBMCs and/or T cells (including increasing secretion of one or more cytokines such as IL-2 and/or IFNγ). The enhancement of immune response can be assessed using a number of in vitro or in vivo measurements known to those skilled in the art, including, but not limited to, cytotoxic T lymphocyte assays, release of cytokines, regression of tumors, survival of tumor bearing animals, antibody production, immune cell proliferation, expression of cell surface markers, and cytotoxicity. Typically, methods of the present application enhance the immune response by a mammal when compared to the immune response by an untreated mammal or a mammal not treated using the recited methods.

In practicing the therapeutic methods, the heterodimeric proteins, multispecific antibodies, or activatable antibodies may be administered alone as monotherapy, or administered in combination with one or more additional therapeutic agents or therapies. Thus, in another aspect, the present application provides a combination therapy, which comprises a heterodimeric protein, multispecific antibody, or activatable antibody described herein in combination with one or more additional therapies or therapeutic agents for separate, sequential or simultaneous administration. The term “additional therapeutic agent” may refer to any therapeutic agent other than a heterodimeric protein, multispecific antibody, or activatable antibody provided by the application.

A wide variety of cancer therapeutic agents may be used in combination with a heterodimeric protein, multispecific antibody, or activatable antibody provided by the present application. One of ordinary skill in the art will recognize the presence and development of other cancer therapies, which can be used in combination with the methods and heterodimeric proteins, multispecific antibodies, or activatable antibodies of the present application, and will not be restricted to those forms of therapy set forth herein. Examples of categories of additional therapeutic agents that may be used in the combination therapy for treating cancer include (1) chemotherapeutic agents, (2) immunotherapeutic agents, and (3) hormone therapeutic agents. In some embodiments, the additional therapeutic is a viral gene therapy, an immune checkpoint inhibitor, a target therapy, a radiation therapies, and/or a chemotherapeutic. In some embodiments, the combination therapy comprises surgery to remove a tumor.

The dosage, dosing frequency, route of administration for the therapeutic methods described herein depend on a number of factors, such as the type, and severity of the disorder to be treated, the particular heterodimeric protein, multispecific antibody or activatable antibody administered, the time of administration, the duration of the treatment, the particular additional therapy administered, the age, sex, weight, condition, general health and prior medical history of the patient being treated, and like factors known in the medical arts.

Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

### EXAMPLES

The examples below are intended to be purely exemplary of the invention and should therefore not be considered to limit the invention in any way. The following examples and detailed description are offered by way of illustration and not by way of limitation.

### Example 1. Design of Fc Domain Mutations

Novel Fc mutations were designed, including disulfide bond mutations, charged mutations, and combinations thereof as shown in Tables 1A and 1B.

TABLE 1A
Novel Fc mutations designed

| | Mutations | CH3 SEQ |
|---|---|---|
| Designs | (first CH3 domain-second CH3 domain) | ID NOs |
| Disulfide | N390C-S400′C | 24, 23 |
| bond | S400C-N390′C | 23, 24 |
| | K392C-V397′C | 25, 26 |
| | V397C-K392′C | 26, 25 |
| | K392C-S400′C | 27, 28 |
| | S400C-K392′C | 28, 27 |
| Charge | E357K:T411K-L351′D:K370′D | 9, 10 |
| designs | E357K:S364K-L351′D:K370′D | 11, 12 |
| | D356K:E357K:S364K-L351′D:K370′D:K439′D | 13, 14 |
| Charge + | E357K:S364K:N390C-L351′D:K370′D:S400′C | 15, 16 |
| disulfide | E357K:S364K:S400C-L351′D:K370′D:N390′C | 17, 18 |
| bond | D356K:E357K:S364K:N390C- | 19, 20 |
| designs | L351′D:K370′D:S400′C:K439′D | |
| | D356K:E357K:S364K:S400C- | 21, 22 |
| | L351′D:K370′D:N390′C:K439′D | |

TABLE 1B
Fc mutations ID.

| Mutations | CH3 SEQ | Mutations |
|---|---|---|
| ID | ID NOs | (first CH3 domain-second CH3 domain) |
| TRF01 | — | T366S, L368A, Y407V, Y349C-T366′W, S354′C |
| TRF02 | — | T350V, L351Y, F405A, Y407V-T350′V, T366′L, |
| | | K392′L, T394′W |
| TRF03 | — | K196Q, S228P, F296Y, E356K, R409K, H435R, |
| | | L445P-K196′Q, S228′P, F296′Y, |
| | | R409′K, K439′E, L445′P |
| TYM01 | 1, 2 | T366S, L368A, Y407V, N390C-T366′W, S400′C |
| TYM02 | 3, 4 | T366S, L368A, Y407V, S400C-T366′W, N390′C |
| TYM03 | — | Y349C, L368V, Y407V-S354′C, T366′W |
| TYM04 | — | L368V, Y407V-T366′W |
| TYM05 | 5, 6 | L368V, Y407V, N390C-T366′W, S400′C |
| TYM06 | 7, 8 | L368V, Y407V, S400C-T366′W, N390′C |
| TYM07 | 9, 10 | E357K:T411K-L351′D:K370′D |
| TYM08 | 11, 12 | E357K:S364K-L351′D:K370′D |
| TYM09 | 13, 14 | D or E356K:E357K:S364K- |
| | | L351′D:K370′D:K439′D |
| TYM10 | 15, 16 | E357K:S364K:N390C-L351′D:K370′D:S400′C |
| TYM11 | 17, 18 | E357K:S364K:S400C-L351′D:K370′D:N390′C |
| TYM12 | 19, 20 | D or E356K:E357K:S364K:N390C- |
| | | L351′D:K370′D:S400′C:K439′D |
| TYM13 | 21, 22 | D or E356K:E357K:S364K:S400C- |
| | | L351′D:K370′D:N390′C:K439′D |

### Example 2. Heterodimer Purity Assessment

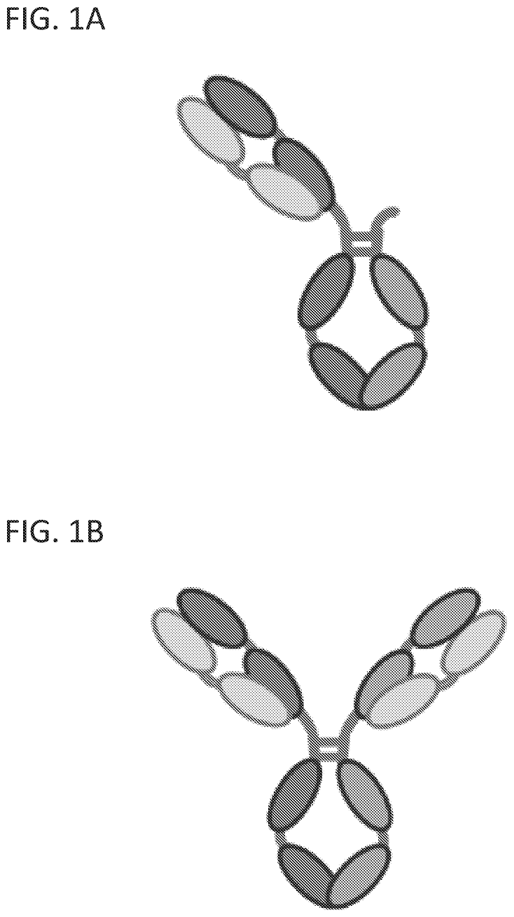

In order to test the novel Fc mutations, heterodimers TYM01 to TYM013 and reference heterodimers were constructed. The corresponding novel Fc mutations are listed in Table 2. A Fab-Fc/Fc one-armed construct was designed with the mutations at the CH3 domain interface (FIG. 1A). In order to evaluate the effect of the mutations on hetero- and homodimerization of the CH3 domain, cloning in mammalian expression vector was performed such that the constructed CH3_A domain could be expressed in a light chain-heavy chain (“LC-HC”) half-body, and the CH3_B domain could be expressed in an Fc-only form. Plasmids encoding light chain (“LC”), heavy chain (“HC”), and Fc at a 2:1:1 molar ratio were co-transfected into HEK293 cells for transient expression. Excess LC over HC chain DNA was used in attempt to avoid the LC from being limiting. Cell culture supernatants were filtered through a 0.45 μm sterile filter. Antibodies were purified by protein A affinity chromatography using HiTrap MabSelect SuRe prepacked columns (GE Healthcare) and were subsequently buffer-exchanged. The products were evaluated by SDS-PAGE and SEC-HPLC to assess the heterodimeric yields.

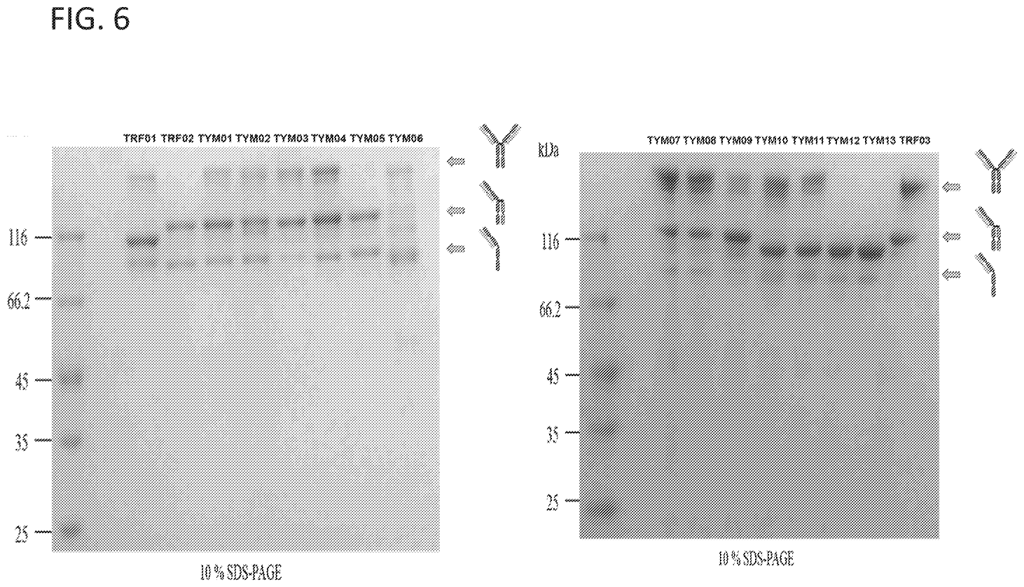

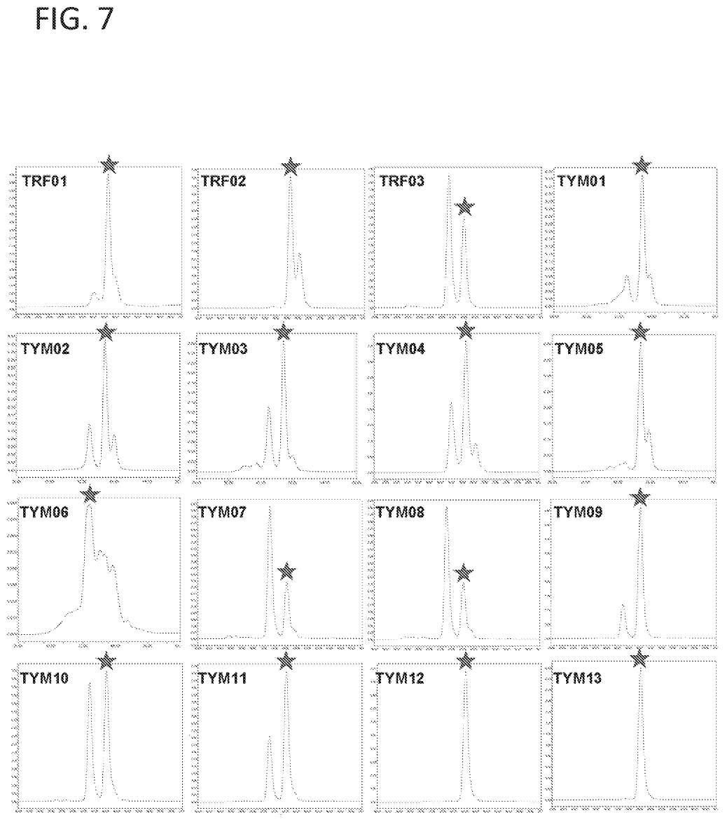

In Fab-Fc/Fc one-armed construct system, the heterodimer and two homodimers have different size and molecular weight, facilitating the identification of the various pairings by SDS-PAGE electrophoresis and size-exclusion high-performance liquid chromatography (“SEC-HPLC”). Proteins were visualized electrophoretically under reducing and non-reducing conditions. Under reducing conditions, three bands were observed, corresponding to HC monomer, Fc monomer, and LC monomer. FIG. 6 shows that under non-reducing conditions there are three bands, corresponding to LC-HC homodimer, LC-HC-Fc heterodimer, and LC-HC half-body. Fc homodimer was not detected under these conditions. Heterodimer yield was also assessed by SEC-HPLC. As shown in FIG. 7, three peaks were detected overall. The first peak in the spectra corresponded to the homodimer, the second peak at 46.6 m corresponded to the heterodimer, and the third peak corresponds to the LC-HC half-body. Quantification of the peak areas in SEC-HPLC enabled identification of variant pairs that stabilize heterodimer relative to homodimer. Purity was calculated using the first peak and second peak, and the results are shown in Table 2.

TABLE 2
Heterodimer purity

| | Mutations | | |
|---|---|---|---|
| ID | ID | Mutations | Purity |
| TY52165 | TRF01 | T366S, L368A, Y407V, Y349C-T366′W, S354′C | 87 |
| TY52166 | TRF02 | T350V, L351Y F405A, Y407V-T350′V, T366′L, K392′L, T394′W | 98 |
| TY52187 | TRF03 | K196Q, S228P, F296Y, E356K, R409K, H435R, L445P-K196′Q, S228′P, | 40 |
| | | F296′Y, R409′K, K439E, L445′P | |
| TY52167 | TYM01 | T366S, L368A, Y407V, N390C-T366′W, S400′C | 69 |
| TY52168 | TYM02 | T366S, L368A, Y407V, S400C-T366′W, N390′C | 68 |
| TY52169 | TYM03 | Y349C, L368V, Y407V-S354′C, T366′W | 62 |
| TY52170 | TYM04 | L368V, Y407V-T366′W | 62 |
| TY52171 | TYM05 | L368V, Y407V, N390C-T366′W, S400′C | 91 |
| TY52172 | TYM06 | L368V, Y407V, S400C-T366′W, N390′C | NA |
| TY52180 | TYM07 | E357K:T411K-L351′D:K370′D | 31 |
| TY52181 | TYM08 | E357K:S364K-L351′D:K370′D | 31 |
| TY52182 | TYM09 | D356K:E357K:S364K-L351′D:K370′D:K439′D | 80 |
| TY52183 | TYM10 | E357K:S364K:N390C-L351′D:K370′D:S400′C | 54 |
| TY52184 | TYM11 | E357K:S364K:S400C-L351′D:K370′D:N390′C | 68 |
| TY52185 | TYM12 | D356K:E357K:S364K:N390C-L351′D:K370′D:S400′C:K439′D | 99 |
| TY52186 | TYM13 | D356K:E357K:S364K:S400C-L351′D:K370′D:N390′C:K439′D | 99 |

### Example 3. Heterodimer Stability Assessment

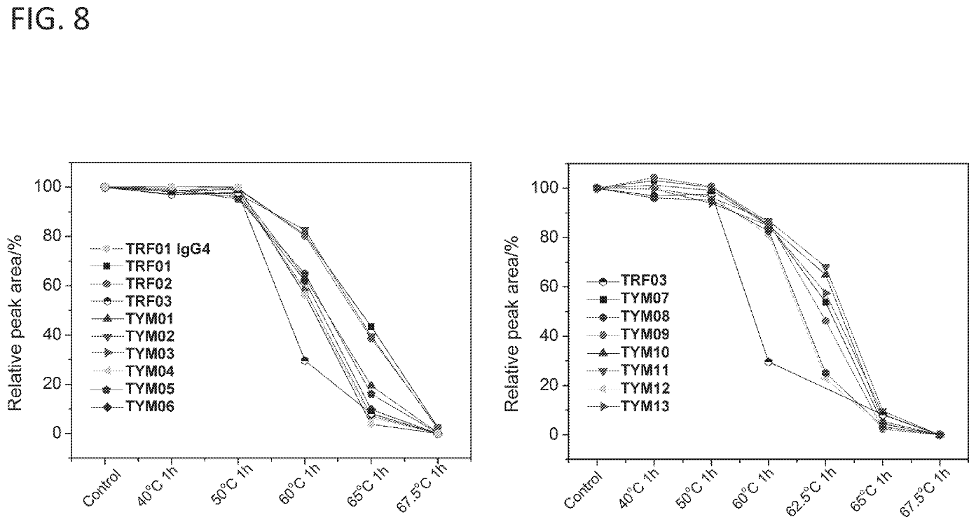

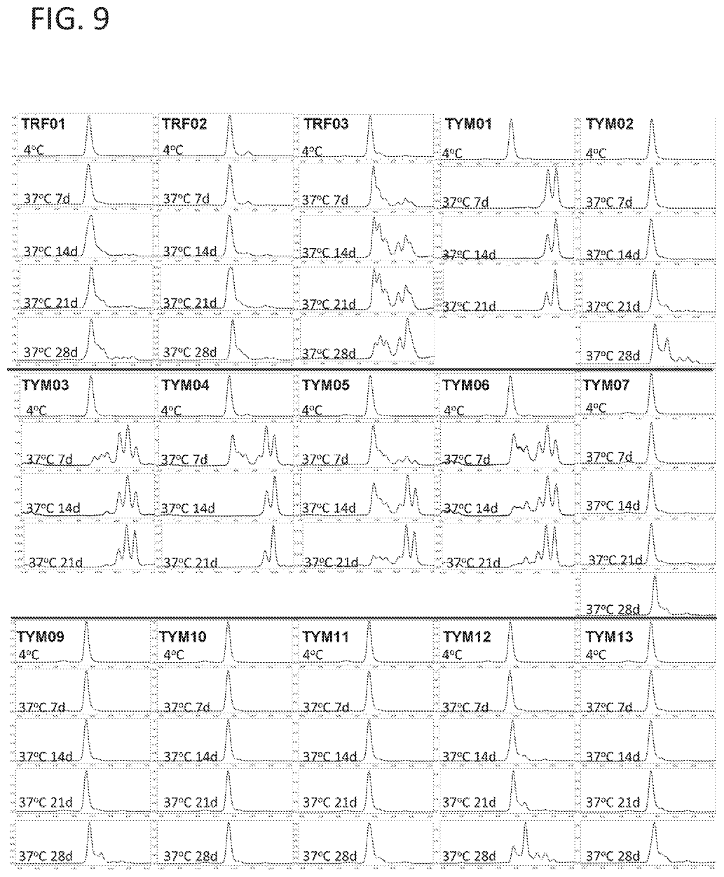

Further, the stability of the heterodimers was assessed by incubation under forced degradation conditions. Purified heterodimer samples were diluted into an appropriate buffer at 1 mg/mL and heated to a different temperature for 1 hour (FIG. 8), or incubated at 37° C. for up to 4 weeks (FIG. 9). The treated samples were analyzed by SEC-HPLC. FIG. 8 shows the different resistance of the proteins aggregation and precipitation at high temperature. The variations of SEC-HPLC spectra after storage at 37 C are shown in FIG. 9. Proteins TYM10, TYM11 and TYM013 showed a relatively superior stability.

### Example 4. Generation of Heterodimers Targeting CD137 and PDL1

Anti-CD137 and anti-PDL1 antibodies were used to construct bispecific antibodies with Fc mutations. The pharmacokinetics of monoclonal antibodies can be modulated by modifying their interactions with the neonatal Fc receptor FcRn. Improving the affinity of the FcRn-IgG interaction can extend the half-life of a modified IgG. FcRn binds to the Fc region of IgG in a strictly pH-dependent manner. At the physiological pH 7.4. FcRn does not bind IgG, but at the acidic pH of the endosome (pH 6-6.5), FcRn has a low micromolar to nanomolar affinity for the Fc region of IgG. FcRn binding characteristics can therefore reflect how the mutations affect PK of the Fc region. Table 3 illustrates that the pH-dependent FcRn binding of bispecific antibodies was not affected at both acidic and physiological pH.

TABLE 3
FcRn binding of bispecific antibodies

| | | pH | FcRn |
|---|---|---|---|
| | | dependent | binding at |
| | | FcRn | acidic |
| ID | Fc mutant | binding/% | pH/nM |
| TY21624 | TRF01 | −0.5 | 46.7 |
| TY21486 | TYM05 | 0.0 | 30.0 |
| TY21487 | TYM09 | 2.2 | 38.2 |
| TY21488 | TYM11 | 0.4 | 33.3 |
| TY21489 | TYM13 | 0.0 | 53.7 |
| TY21625 | TYM10 | 2.4 | 26.3 |

Anti-CD137 and anti-PDL1 antibodies with common light chains were used to construct bispecific antibodies with common light chains and different Fc mutations (FIG. 1B). The bispecific antibodies were also tested in a 293T-CD137-NFκB reporter assay. In brief, 50×10⁴/ml of 293T-CD137 cells and 50×10⁴/ml of 293T-PDL1 cells were mixed together, and the mixed cells were then split into wells of a 96-well plate at a density of 5×10⁴ cells/well (100 μl/well). 50 μl of diluted antibody solution was added into corresponding wells and incubated for 18 h. After incubation, the medium was aspirated and then 50 μl of Passive Lysis Buffer (Promega E1980) was added and incubated at 37° C. for 30 minutes. 20 μl supernatant was transferred to the white plate (Costar, 3912) and then 40 μl of firefly substrate and 40 μl of Renina substrate were added, and the luminescence signal was read (Promega E1980).

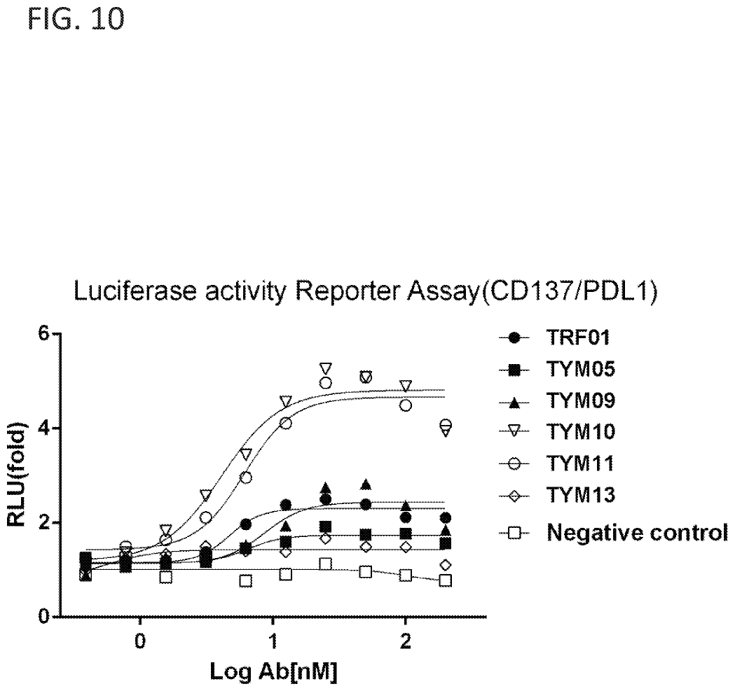

As shown in FIG. 10, TYM10 and TYM11 had relatively higher activities in the NFκB reporter assay.

### Example 5. Generation and Characterization of Bispecific Antibody Targeting CD137 and PDL1

### A. Generation of Bispecific Antibody

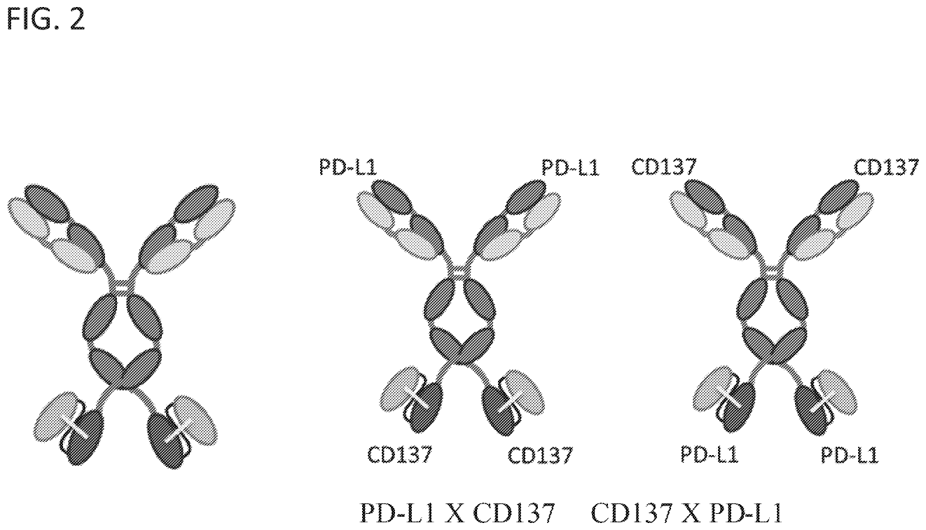

The following example describes the development of a developable and effective format for a bispecific antibody targeting CD137 and PDL1. The format was optimized on the basis of a “Morrison format” (FIG. 2). DNAs encoding anti-CD137 Fv and anti-PDL1 Fv were used to construct an expressing plasmid in the form of Fab or scFv. The scFv has a reduced affinity compared to Fab, so the orientation of the two Fvs (i.e., CD137×PDL1 or PDL1-CD137 in the form of Fab×scFv) could affect efficacy of the bispecific antibodies.

IgG1 or IgG4 (S228P) isotypes were employed. The scFv was linked to the C-terminus of the Fc in the VH-to-VL orientation by a linker of SGGGS (SEQ ID NO: 80) or GGGSGGGGS (SEQ ID NO: 81). Among the scFv, the C-terminus of VH was linked to the N-terminus of VL by a (G₄S)₄ (SEQ ID NO: 82) linker. A VH-44 to VL-100 disulfide bond was also incorporated into the scFv, designed to stabilize the format. A pair of novel engineered N390C_{CH3A}-S400′C_{CH3B} disulfide bonds in CH3 domain were also tested (SEQ ID NOs: 23-24). In addition, an N297A mutation was introduced to silence the effector function mediated by the Fc region. Table 4 provides the eight scaffold designs that were developed. Table 5 lists SEQ ID NOs. corresponding to the first heavy chain, first light chain, second heavy chain, second light chain of exemplary CD137×PDL1 and PDL1×CD137 antibodies.

TABLE 4
Scaffold designs for bispecific antibodies

| | | N390C: | | Fc-scFv | SEQ | scFv | scFv |
|---|---|---|---|---|---|---|---|
| Format | Isotype | S400′C | N297A | Linker | ID No. | Linker | Disulfide bond |
| TYF01 | IgG1 | No | No | SGGGS | 80 | (G₄S)₄ | VH44-VL100 |
| TYF02 | IgG1 | Yes | No | SGGGS | 80 | SEQ ID NO: 82 | |
| TYF03 | IgG1 | No | Yes | SGGGS | 80 | | |
| TYF04 | IgG1 | Yes | Yes | SGGGS | 80 | | |
| TYF05 | IgG1 | Yes | Yes | GGGSGGGGS | 81 | | |
| TYF06 | IgG4(S228P) | No | No | SGGGS | 80 | | |
| TYF07 | IgG4(S228P) | Yes | No | SGGGS | 80 | | |
| TYF08 | IgG4(S228P) | Yes | No | GGGSGGGGS | 81 | | |

### B. CMC Characterizations of Bispecific Antibody

Plasmids encoding heavy and light chains of bispecific antibodies were transiently transfected into mammalian cells. Bispecific antibody-containing cell culture supernatants were harvested 7 days after transfection by centrifugation at 14000 g for 30 min and were filtered through a sterile filter (0.22 μm). Antibodies were purified by protein A affinity chromatography using MabSelect SuRe prepacked columns (GE Healthcare) and were subsequently buffer exchanged in 20 mM histidine (pH 5.5) buffer.

The aggregation ratio of purified bispecific antibodies after purification was evaluated by analytical size-exclusion chromatography (“SEC”). The analysis was performed as follows: SEC was performed on a Waters 2695 combined with a Waters 2996 UV detector. A TSK Gel g3000 SWXL column (300 mm×7.8 mm) with a TSK Gel g3000 SWXL pre-column (Tosoh Bioscience) was used. 10 μg of each sample was injected and separation was performed at a flow rate of 0.5 mL/min. The elution buffer was composed of 200 mM sodium phosphate at pH 7.0. UV detection was performed at 214 nm. The freeze-thaw stability was studied by freezing 100 μL sample (1 mg/mL) at −80° C. for 30 min, followed by thawing at room temperature for 60 min. Six freeze-thaw cycles were conducted and the aggregation ratios were also determined by analytical size-exclusion chromatography.

Table 5 provides the yields of the bispecific antibodies after purification, and Table 6 provides aggregation ratios of the bispecific antibodies after purification and freeze-thaw. TYF05 was the best format, with low aggregation formation during expression and no aggregation tendency during freeze-thaw process. The disulfide bond in CH3 and the replacement of SGGGS (SEQ ID NO: 80) linker with a nine-amino-acid-linker GGGSGGGGS (SEQ ID NO: 81) both improved the colloidal stability.

TABLE 5
Yield of the bispecific antibodies after purification

| | PDL1 × CD137 bispecific antibody | | | CD137 × PDL1 bispecific antibody | | |
|---|---|---|---|---|---|---|
| | | Antibody | Yield after | | Antibody | Yield after |
| | | chain SEQ | purification | | chain SEQ | purification |
| Format | ID | ID NOs. | (mg/L) | ID | ID NOs. | (mg/L) |
| TYF01 | TY22121 | 84, 85 | 54.3 | TY22122 | 96, 97 | 45.4 |
| TYF02 | TY22148 | 86, 87 | 36.6 | TY22149 | 98, 99 | 42.2 |
| TYF03 | TY22172 | 88, 89 | 31.9 | TY22173 | 100, 101 | 47.7 |
| TYF04 | TY22176 | 90, 91 | 28.9 | TY22177 | 102, 103 | 21.6 |
| TYF05 | NA | | NA | TY22359 | 104, 105 | 30.5 |
| TYF06 | TY22161 | 92, 93 | 33.0 | TY22162 | 106, 107 | 31.0 |
| TYF07 | TY22165 | 94, 95 | 35.6 | TY22166 | 108, 109 | 39.3 |
| TYF08 | NA | | NA | TY22362 | 110, 111 | 26.7 |

TABLE 6
Aggregation ratios of the bispecific antibodies after purification and freeze-thaw

| | PDL1 × CD137 bispecific antibody | | | CD137 × PDL1 bispecific antibody | | |
|---|---|---|---|---|---|---|
| | (Aggregation ratio % ) | | | (Aggregation ratio %) | | |
| | | | After | | | after |
| | | After | freeze- | | after | freeze- |
| Formats | ID | purification | thaw | ID | purification | thaw |
| TYF01 | TY22121 | 2.1 | 9.1 | TY22122 | 8.4 | 10.8 |
| TYF02 | TY22148 | 1.7 | 5.4 | TY22149 | 4.5 | 8.5 |
| TYF03 | TY22172 | 2.6 | 3.8 | TY22173 | 10.2 | 9.3 |
| TYF04 | TY22176 | 3.3 | 4.6 | TY22177 | 5.1 | 7.6 |
| TYF05 | NA | NA | NA | TY22359 | 5.8 | 5.4 |
| TYF06 | TY22161 | 3.5 | 3.5 | TY22162 | 10.8 | 11.3 |
| TYF07 | TY22165 | 2.3 | 2.1 | TY22166 | 8.8 | 8.9 |
| TYF08 | NA | NA | NA | TY22362 | 7.8 | 8.2 |

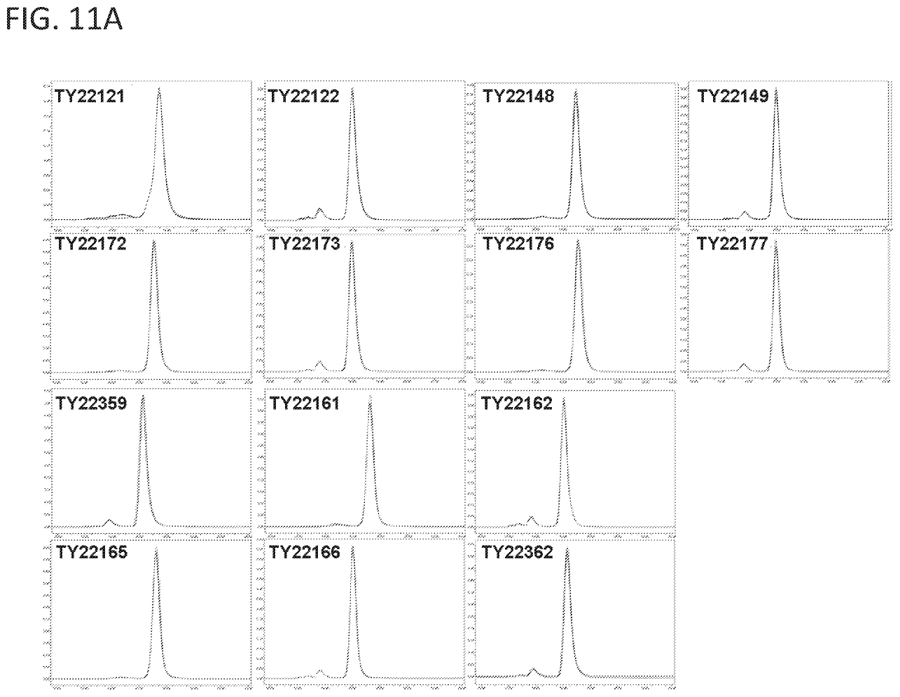

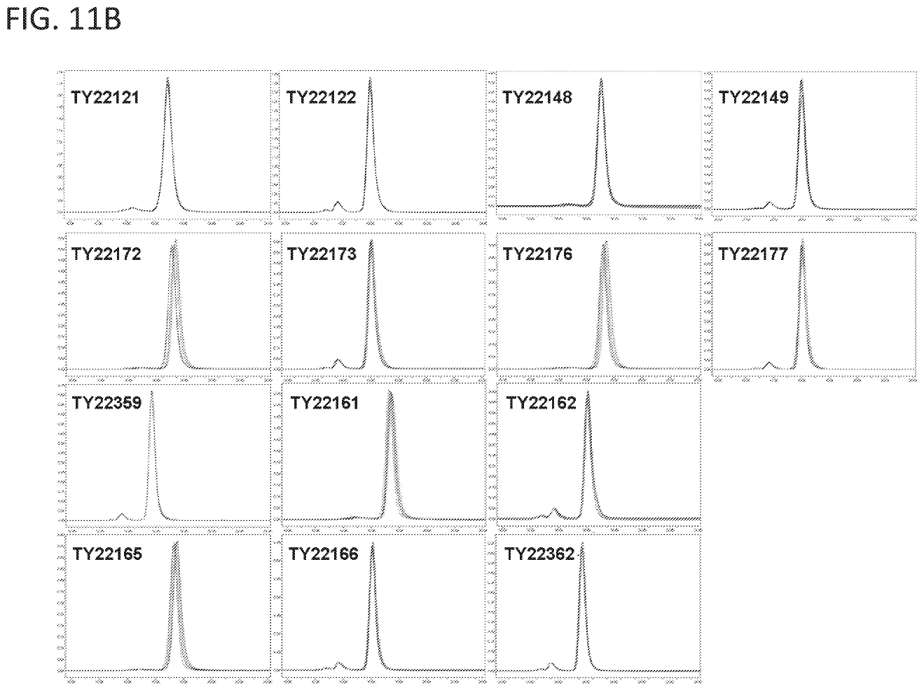

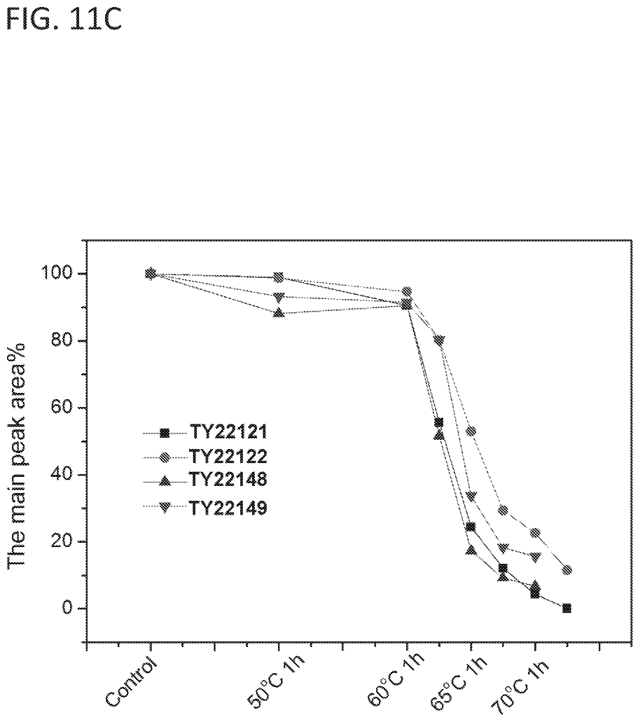

Freeze and thaw for 6 cycles were tested by using purified proteins at 1 mg/mL, also incubated at 40° C. for 28 days (FIGS. 11A-11B). Four purified proteins were also heated at high temperature to confirm their thermal stability (FIG. 11C). FIGS. 11A-11B show the protein quality evaluated by analytical size-exclusion chromatography. All formats have little aggregation and degradation under accelerated storage condition that indicate they have a good long-term storage stability. As shown in FIG. 11C, all tested proteins aggregate and precipitate upon 60° C., and CD137×PDL1 bispecific antibody had a better colloidal stability than PDL1×CD137 bispecific antibody. These data pointed toward CD137×PDL1 as an antibody format most suitable for targeting CD137 and PDL1.

### C. Binding Affinity of Bispecific Antibody

Biacore T200 (GE Healthcare) was used as a high performance system for real-time biomolecular interaction analysis, using surface plasmon resonance technology (“SPR”). During the measurement, anti-human IgG monoclonal antibody from the Human Antibody Capture Kit provided by Biacore was immobilized on CM5 chips, and IgG sample was injected onto sensor chip. Analytes were injected onto IgG captured flow cell for binding kinetic analysis in HBS-EP buffer. The data was fitted according to 1:1 Langmuir model and the K_{D} value determined (Table 7). Format 5 had the highest affinity among all bispecific antibodies.

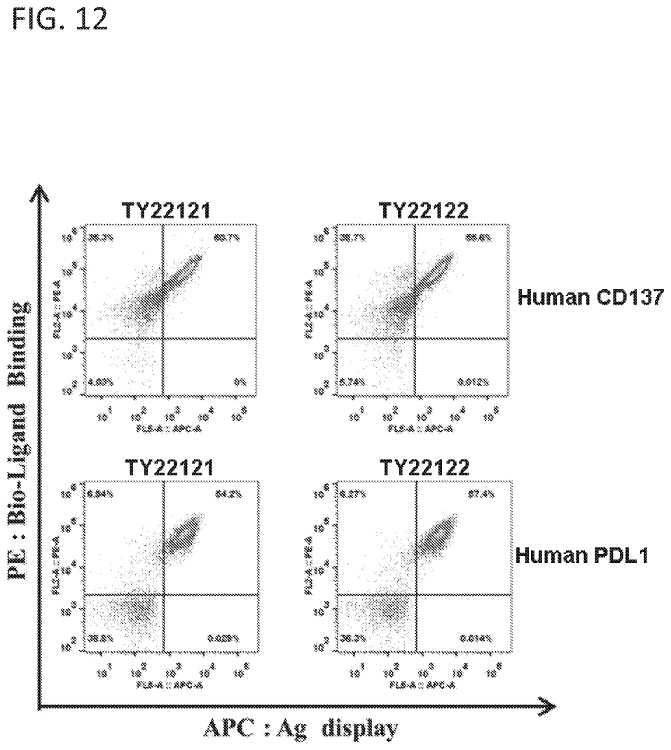

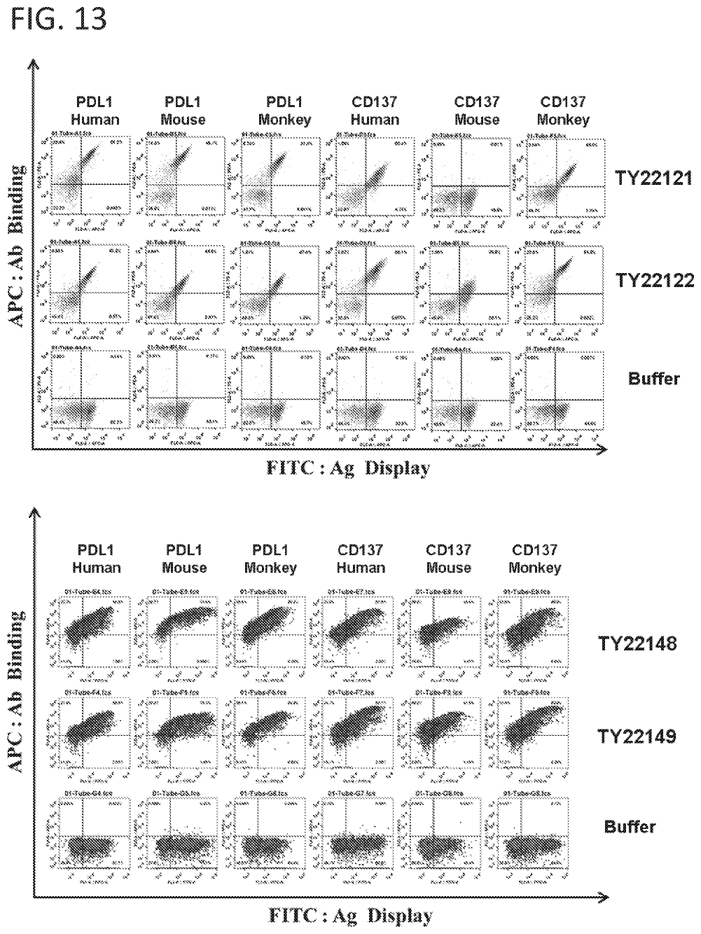

The affinity of antibodies was also assessed against human, monkey and mouse CD137 or PDL1 that were transiently expressed on the surface of yeast or HEK293F cells. Briefly, yeast or HEK293F cells were transfected with a plasmid expressing human, monkey or mouse CD137 or PDL1. After 48 hours, the transfected cells were harvested and then washed. Cells were then incubated with IgGs (each at 100 nM) at 4° C. in the shaking bed for 1 hour shaking at 300 rpm, protected from light. For simultaneously binding, cell were incubated with biotinylated human CD137 or PDL1 protein fused with human Fc fragment, and SA-PE (streptavidin, phycoerythrin conjugated). For cross-reactivity, cells were incubated with Alexa Fluor 647 conjugated mouse anti-human Fc antibodies. The mixtures were incubated at 4° C. in the shaking bed for 30 minutes shaking at 300 rpm, protected from light. The cells were washed once prior to analysis by flow cytometry (Beckman CytoFlex). FIG. 12 shows that the bispecific antibody simultaneously binds human PDL1 and CD137. In addition, FIG. 13 shows that the bispecific antibodies maintained parental cross-reactivity with PDL1 or CD137 of human, mouse, or monkey origin.

TABLE 7
Binding affinity to PDL1H and CD137H

| | PDL1 × CD137 bispecific antibody | | | CD137 × PDL1 bispecific antibody | | |
|---|---|---|---|---|---|---|
| | | Human | Human | | Human | Human |
| | | PDL1 | CD 137 | | PDL1 | CD137 |
| Formats | ID | (nM) | (nM) | ID | (nM) | (nM) |
| TYF01 | TY22121 | 6.9 | 14.1 | TY22122 | 29.7 | 3.8 |
| TYF02 | TY22148 | 7.7 | 6.7 | TY22149 | 32.0 | 2.4 |
| TYF04 | TY22176 | 8.1 | 7.1 | TY22177 | 33.4 | 7.0 |
| TYF05 | NA | NA | NA | TY22359 | 29.8 | 2.7 |
| TYF07 | TY22165 | 7.7 | 5.2 | TY22166 | 42.0 | 3.0 |
| TYF08 | NA | NA | NA | TY22362 | 40.3 | 2.8 |

### D. In Vitro and In Vivo Efficacy

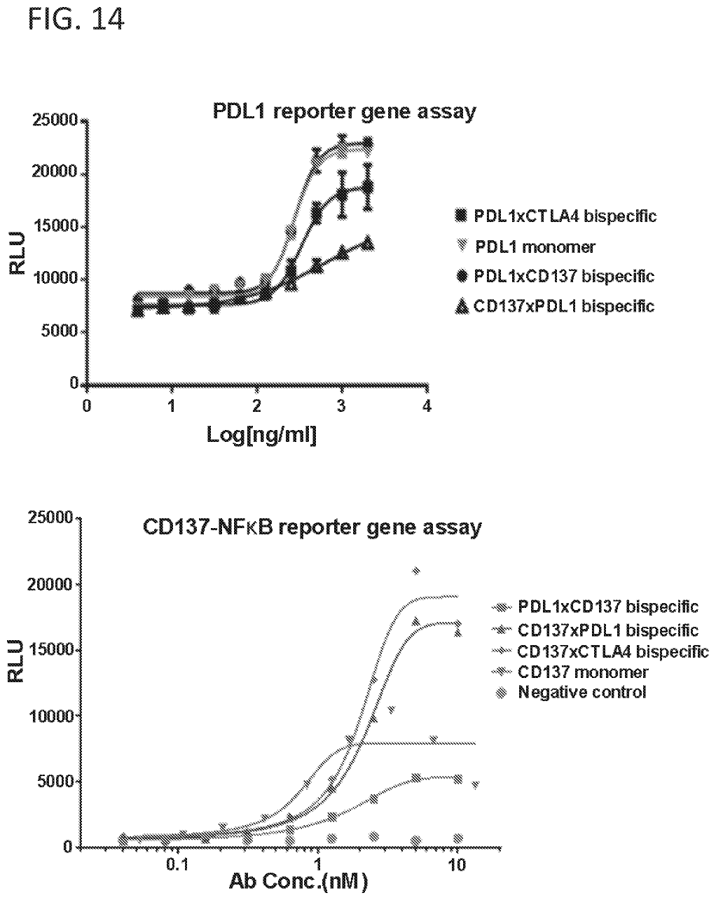

The effect of PDL1×CD137 and CD137×PDL1 bispecific antibodies on in vitro reporter gene assays was tested (FIG. 14). Anti-PDL1-based bispecific antibodies were assessed by PDL1 blockade bioassay. In brief, Jurkat T cells expressing human PD-1 and a luciferase reporter driven by an NFAT response element (NFAT-RE) were used as PD-1 effector cells. CHO-K1 cells expressing human PDL1 and an engineered cell surface protein designed to activate cognate TCRs in an antigen-independent manner were used as PDL1 aAPC/CHO-K1 cells. PD-1 effector cells were incubated with PDL1 aAPC/CHO-K1 cells in the absence or presence of anti-PDL1-based bispecific antibodies or PDL1 monomer blocking antibody, as indicated in FIG. 14. BIO-GLO™ Reagent was added and luminescence quantified. Data were analyzed using GraphPad Prism® software. Anti-CD137-based bispecific antibodies were assessed by NFκB reporter assay. CD137-NFκB-293T stable cells were restored, cultured and split to the 96-well plate (50 μL/well, at density of 6×10⁵). After 5.5 hours of incubation, diluted test antibodies (indicated in FIG. 14) premixed at 1:5 ratio with a crosslinker were added. Luciferase levels were measured 18 hours later. Relative luciferase units (RLU) against the blank control (without antibody treatment) were normalized for transfection efficiency using Renilla luciferase activity and the results were expressed as mean±standard error in triplicate.

As shown in the top panel of FIG. 14, In PDL1 reporter gene assays show that PDL1×CD137 bispecific antibody had similar activity with PDL1 monomer, and both were stronger than CD137×PDL1 bispecific antibody. Similarly, in the bottom panel of FIG. 14, a CD137 reporter gene assay, CD137×PDL1 bispecific antibody had stronger activity than PDL1×CD137 bispecific antibody. These results indicated that Fab had better activity than scFv. Without wishing to be bound by theory, this may have been because of the difference in affinities.

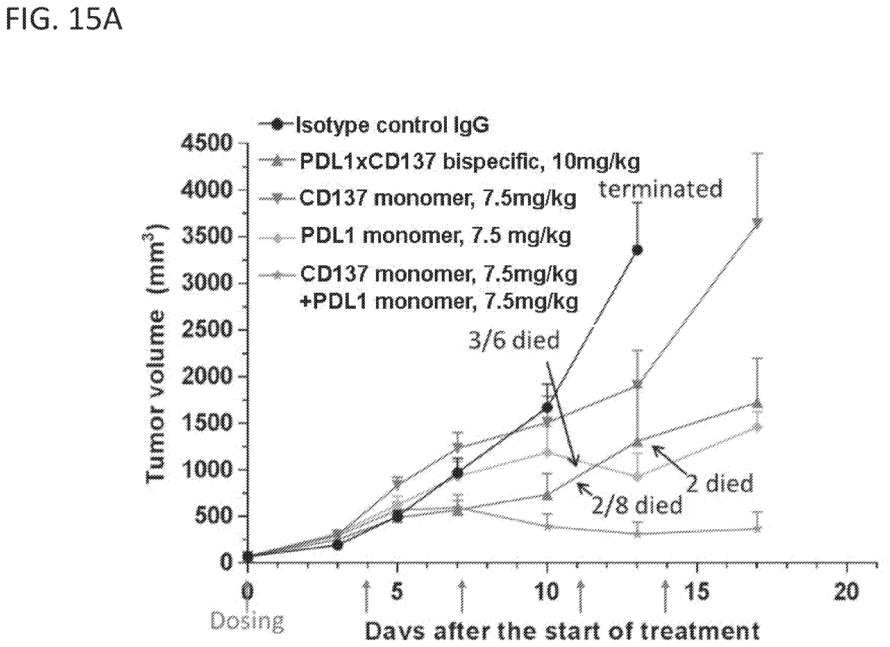

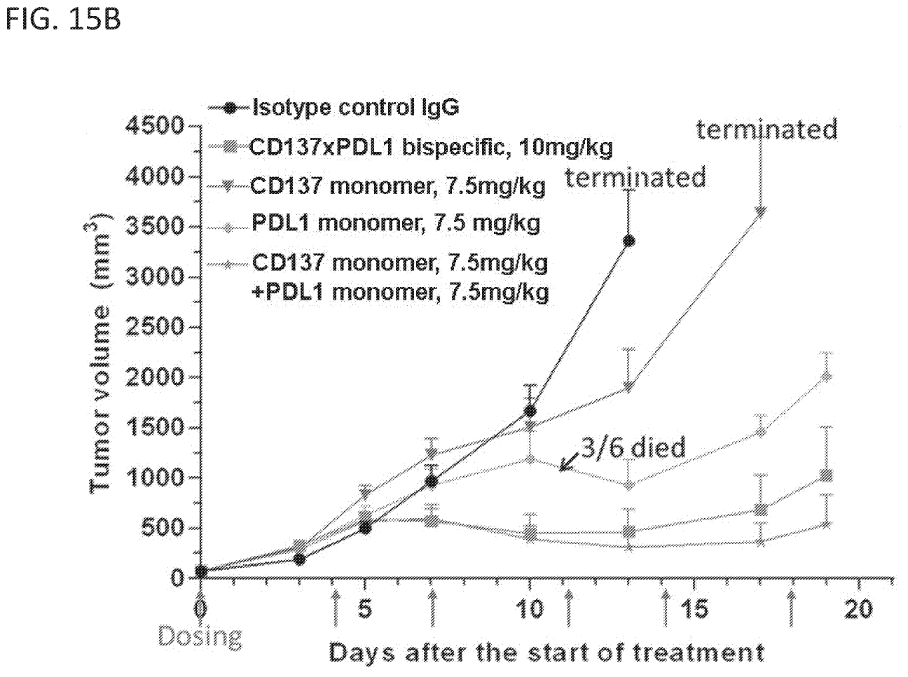

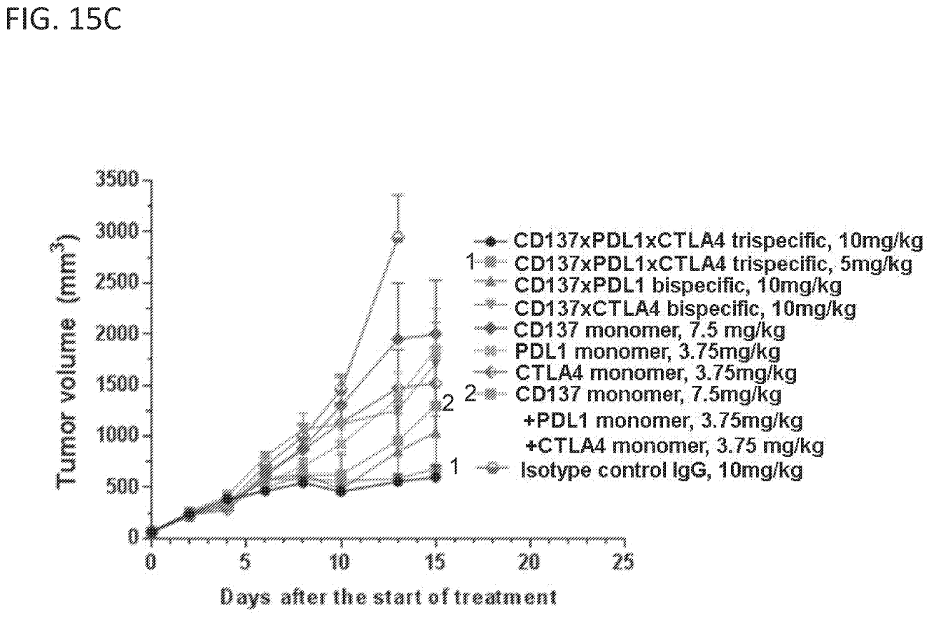

Because the anti-CD137 and anti-PDL1 Fv cross-reacted with mouse and monkey antigens, the in vivo efficacy of bispecific antibodies was studied in the 3LL syngeneic mouse tumor model (FIGS. 15A-15C). Both PDL1×CD137 and CD137×PDL1 bispecific antibodies inhibited tumor growth. CD137×PDL1 bispecific antibody was slightly less efficacious than the combination of CD137 and PDL1 parental antibodies, and much more efficacious than one of the two parental antibodies alone. The PDL1×CD137 bispecific antibody was not as effective as the CD137×PDL1 antibody, indicating that the orientation of the two antigens in this bispecific format is important for efficacy.

### Example 6. Generation of Trispecific Antibodies

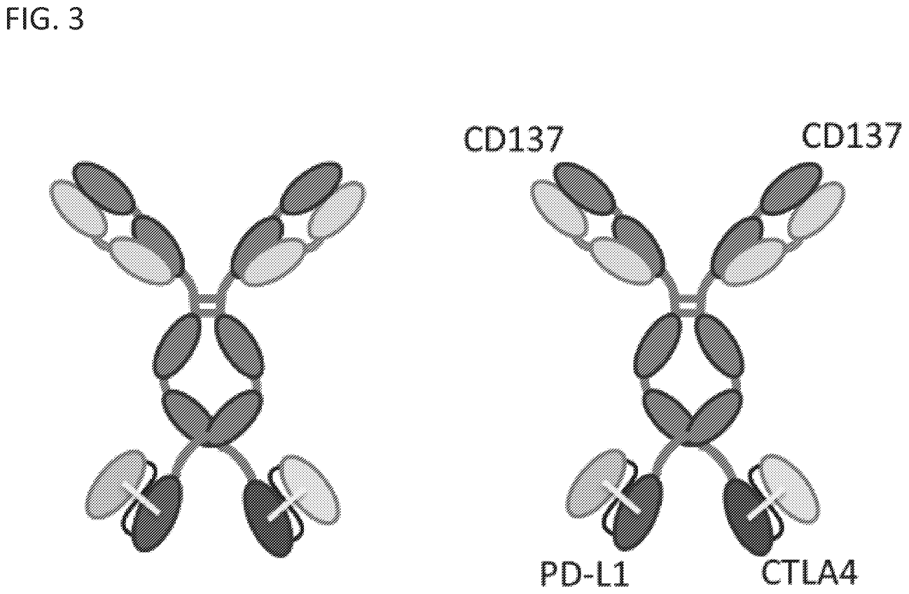

The following example provides trispecific antibodies capable of binding CD137, PDL1, and CTLA4 (FIG. 3).

Three trispecific antibodies combining Fc mutant TYM11 and bispecific formats TYF01, TYF02 and TYF04 were constructed and purified, as shown in Table 8. TYF02 showed the best quality and all the three formats were stable under freeze-thaw and 40° C. storage.

Anti-CD137 and anti-PDL1 Fv cross-reacted with mouse and monkey antigens, so the in vivo efficacy of bispecific antibodies was studied in the 3LL syngeneic mouse tumor model. C57BL/6 mice were transplanted subcutaneously with 2×10⁶ 3LL lung cancer cells. When the tumors were established (70 mm³), treatment began with isotype control IgG (n=6), PDL1×CD137 bispecific (10 mg/kg, n=8). CD137×PDL1 bispecific (10 mg/kg, n=8), CD137 monomer (7.5 mg/kg, n=6), PDL1 monomer (7.5 mg/kg, n=6) or CD137 monomer+PDL1 monomer (both 7.5 mg/kg, n=8) by intraperitoneal injection, for up to 6 doses. Tumor growth was monitored thrice weekly and reported as mean tumor volume±SEM over time. As shown in FIGS. 15A-15B, both PDL1×CD137 and CD137×PDL1 bispecific antibodies inhibited tumor growth. CD137×PDL1 bispecific antibody was slightly less efficacious than the combination of CD137 and PDL1 parental antibodies, and much more efficacious than one of the two parental antibodies alone. The PDL1×CD137 bispecific antibody was not as effective as the CD137×PDL1 antibody, indicating that the orientation of the two antigens in this bispecific format is important for efficacy.

The TYF02 trispecific antibody was also chosen to test in vivo efficacy using the 3LL syngeneic mouse tumor model. C57BL/6 mice were transplanted subcutaneously with 2×10⁶ 3LL lung cancer cells. When the tumors were established (65 mm³), treatment began with isotype control IgG (n=6), CD137×PDL1×CTLA4 trispecific (10 mg/kg, n=8). CD137×PDL1×CTLA4 trispecific (5 mg/kg, n=8), CD137×PDL1 bispecific (10 mg/kg, n=6), CD137×CTLA4 bispecific (10 mg/kg, n=6), CD137 monomer (7.5 mg/kg, n=6). PDL1 (3.75 mg/kg, n=6), CTLA4 (3.75 mg/kg, n=6) or CD137 monomer (7.5 mg/kg)+PDL1 (3.75 mg/kg)+CTLA4 (3.75 mg/kg) (n=6) by intraperitoneal injection, for up to 5 doses. Tumor growth was monitored every two days and reported as mean tumor volume f SEM over time. As shown in FIG. 15C, this trispecific antibody showed better tumor growth inhibition than the corresponding bispecific antibodies or the parental combination of three mono-IgGs.

TABLE 8
Trispecific antibody chemistry, manufacturing and controls

| | | | Antibody | Yield after | | |
|---|---|---|---|---|---|---|
| | | Fc | chain SEQ | purification | QC | QC |
| ID | Format | mutant | ID NOs. | mg/L | HMW(%) | LMW % |
| TY22224 | TYF01 | TYM11 | 118-120 | 28.1 | 2.4 | 6.7 |
| TY22225 | TYF02 | TYM11 | 121-123 | 18.8 | 2.0 | 1.3 |
| TY22226 | TYF04 | TYM11 | 124-126 | 9.9 | 0.9 | 0.3 |

### Example 7. Biophysical Characterization of Heterodimeric HER2×CD3 T-Cell-Engaging Bispecific Antibody

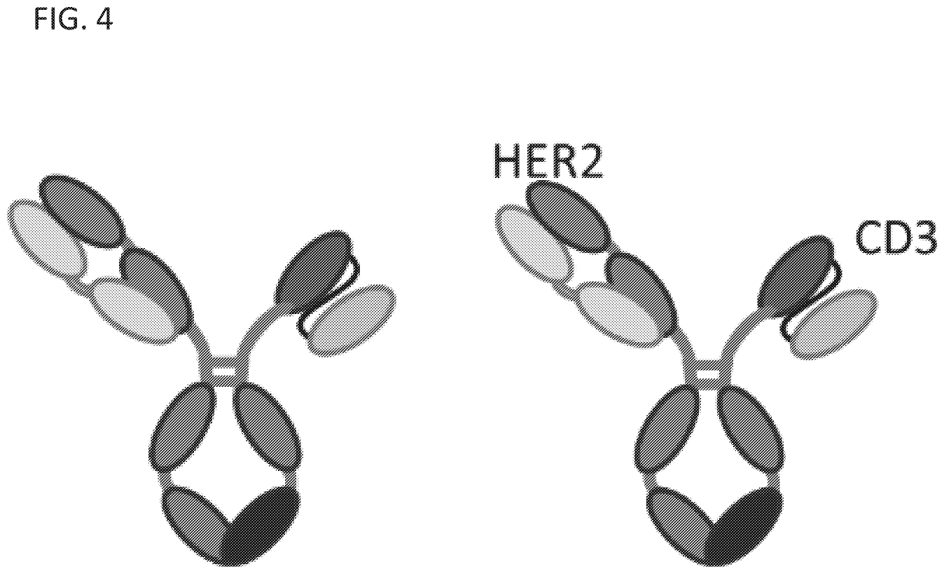

A heterodimeric bispecific scaffold was designed using the TYM13 Fc mutant. A light chain-heavy chain half antibody and a scFv-Fc chain were combined to form a bispecific antibody, with TYM13 mutations in hetero-Fc domain (FIG. 4). A HER2×CD3 bispecific T-cell-engaging antibody was constructed using this scaffold. For comparison, corresponding antibodies having knobs-into-holes mutations Y394C, T366S, L368A, Y407V-S354′C T366′W and “Xencor mutations” E357Q, S364K-L368′D, K370'S were also constructed.

Plasmids encoding the heavy chain, light chain, and scFv-Fc chain of bispecific antibodies were transiently transfected into mammalian cells. Bispecific antibody-containing cell culture supernatants were harvested 7 days after transfection by centrifugation at 14000 g for 30 min and were filtered through a sterile filter (0.22 μm). Antibodies were purified by protein A affinity chromatography using MabSelect SuRe prepacked columns (GE Healthcare) and were subsequently buffer exchanged in 20 mM histidine (pH 5.5) buffer.

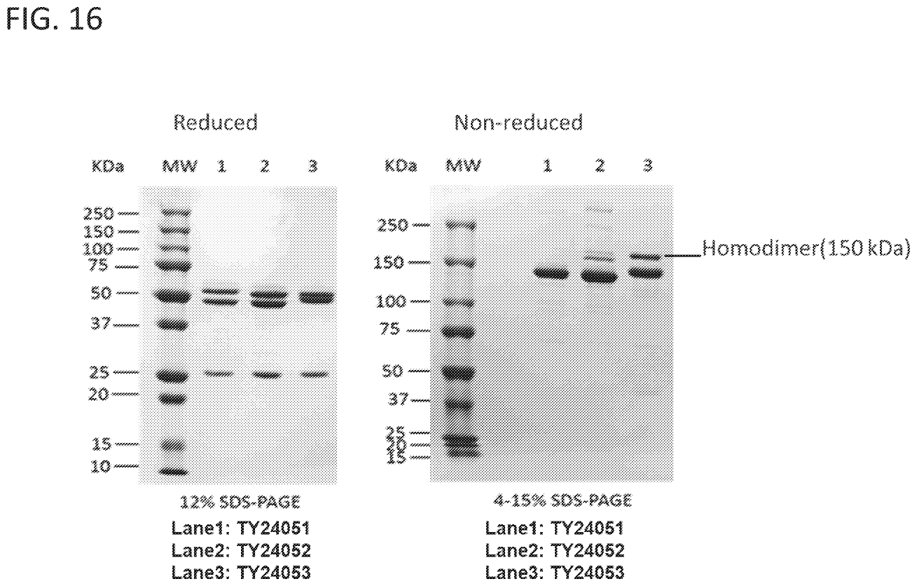

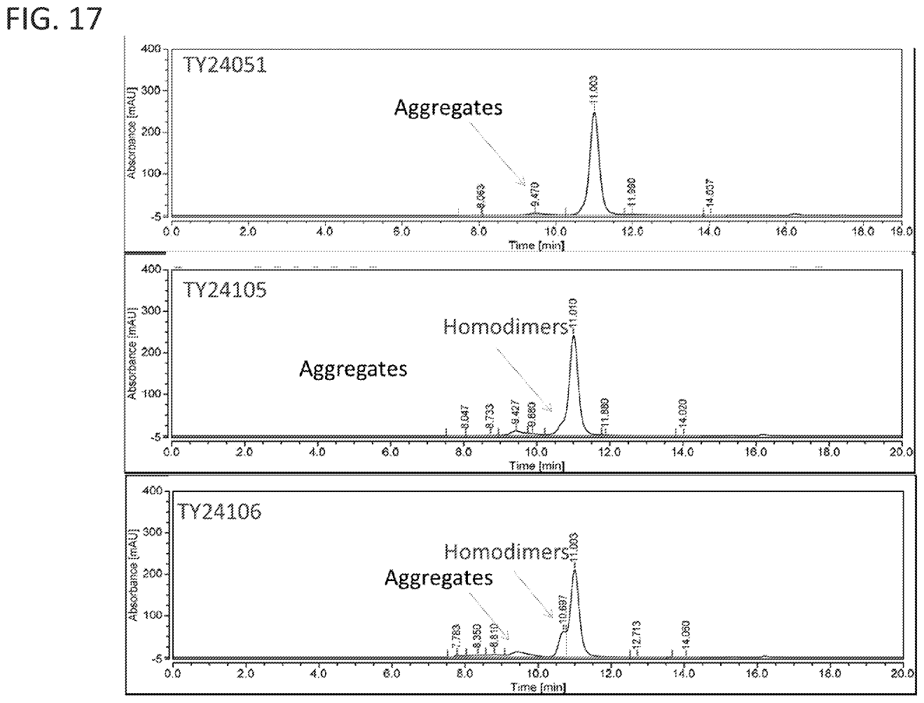

The biophysical purity of heterodimeric bispecific antibodies was assessed through SEC-HPLC and SDS-PAGE. As shown in FIG. 16 and FIG. 17, TY24051 with TYM13 mutations showed very good heterodimeric purity, with no detectable homodimers, while TY24105 and TY24106, with knobs-into-holes and Xencor mutation, both contained some 150 kDa homodimers (indicated in SDS-PAGE and SEC-HPLC graphs in FIG. 16 and FIG. 17, respectively). Furthermore, TY24051 contained fewer aggregates than TY24105 and TY24106.

When TY24051 was converted into an activatable antibody, TY24052, some aggregates were generated (see Table 9). TY24052 could be purified by cation exchange chromatography (CEX).

TABLE 9
Bispecific antibodies and their SEC-HPLC purity

| | | Antibody | SEC-HPLC purity | | |
|---|---|---|---|---|---|
| | | chain SEQ | HMW | Monomer | LMW |
| IgG ID | Fc mutant | ID NOs. | (%) | (%) | (%) |
| TY24051 | TYM13 | 112, 113, | 3.1 | 96.0 | 0.9 |
| | | 114 | | | |
| TY24105 | Knobs-into-holes | — | 9.2 | 89.2 | 1.5 |
| TY24106 | Xencor mutation | — | 31.6 | 68.2 | 0.2 |

### Example 8. Activatable Bispecific Antibody Construction and Functional Characterization

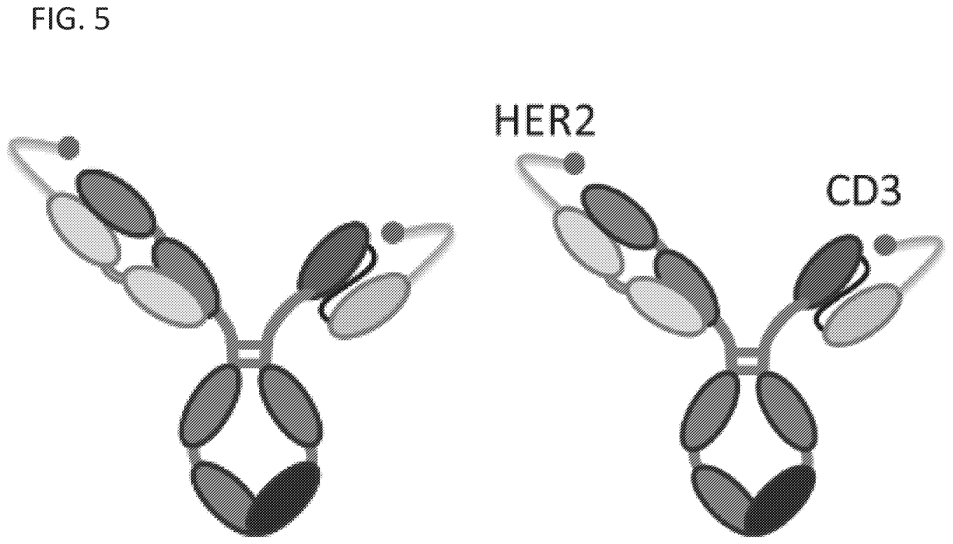

An activatable HER2×CD3 bispecific antibody (also referred herein as “SAFEbody” or “SAFE-bispecific”) was constructed (FIG. 5). The constructs are described in Tables 10 and 11.

TABLE 10
Bispecific antibodies and their purity determined by SEC-HPLC

| | | Antibody | SEC-HPLC purity | | |
|---|---|---|---|---|---|
| | | chain SEQ | HMW | Monomer | LMW |
| IgG ID | Format | ID NOs. | (%) | (%) | (%) |
| TY24051 | TYM13 N297A | 112, 113, | 3.1 | 96.0 | 0.9 |
| | | 114 | | | |
| TY24052 | TYM13 N297A, | 115, 116, | 9.3 | 88.2 | 2.5 |
| | SAFE-bispecific | 117 | | | |

TABLE 11
Design of HER2 × CD3 bispecific SAFEbody

| | | | | | Fc | |
|---|---|---|---|---|---|---|
| | SAFEbody | | | Cleavable | heterodimeric | Fc |
| IgG ID | Motifs | Left Arm | Right Arm | activation | motif | effector |
| TY24051 | Parental | Fab | scFv | NA | TYM13 | N297A |
| | bispecific | (HER2) | (CD3) | | | |
| | Fab X scFv | | | | | |
| | in IgG1 | | | | | |
| TY24052 | Safe | SAFE | SAFE | Cleavable | TYM13 | N297A |
| | Bispecific | Fab | scFv | | | |
| | Fab X scFv | (HER2) | (CD3) | | | |
| | in IgG1 | | | | | |
| TY24053 | Safe | SAFE | SAFE | Non | TYM13 | N297A |
| | Bispecific | Fab | scFv | cleavable | | |
| | Fab X scFv | (HER2) | (CD3) | | | |
| | in IgG1 | | | | | |

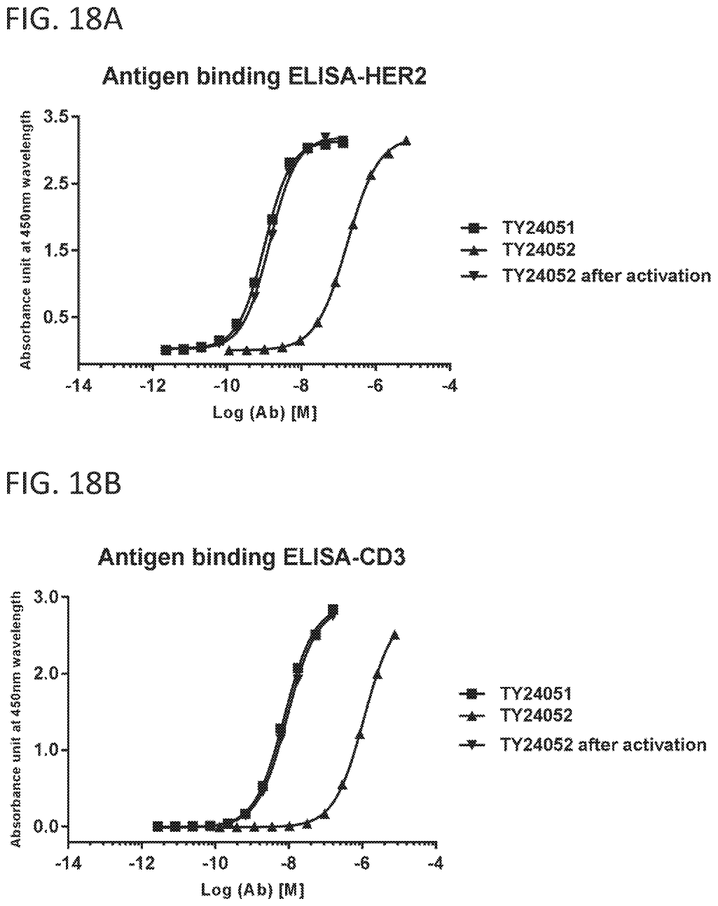

The affinities of the bispecific antibody (TY24051) and its SAFEbody version (TY24052) were analyzed through enzyme-linked immunosorbent assay (ELISA) assay. 2 μg/mL of human HER2 or CD3 (E and 6 chain heterodimer) fused with human Fc fragment, were prepared and used to coat the ELISA plate at 2-8′C overnight. After washing and blocking, 50 μL serial diluted IgGs were added and incubated at 37′C for 1 hour. Plates were washed three times and then incubated with 50 μL/well TMB substrate at room temperature for about 20 minutes. Absorbance at 450 nm was measured after the reaction was stopped. The data was analyzed by GraphPad Prism 6 with nonlinear fitting. As shown in FIGS. 18A-18B, TY24051 bound to both HER2 and CD3, while TY24052 showed an apparently lower affinity than TY24051. After activation, the affinity of TY24052 was fully recovered.

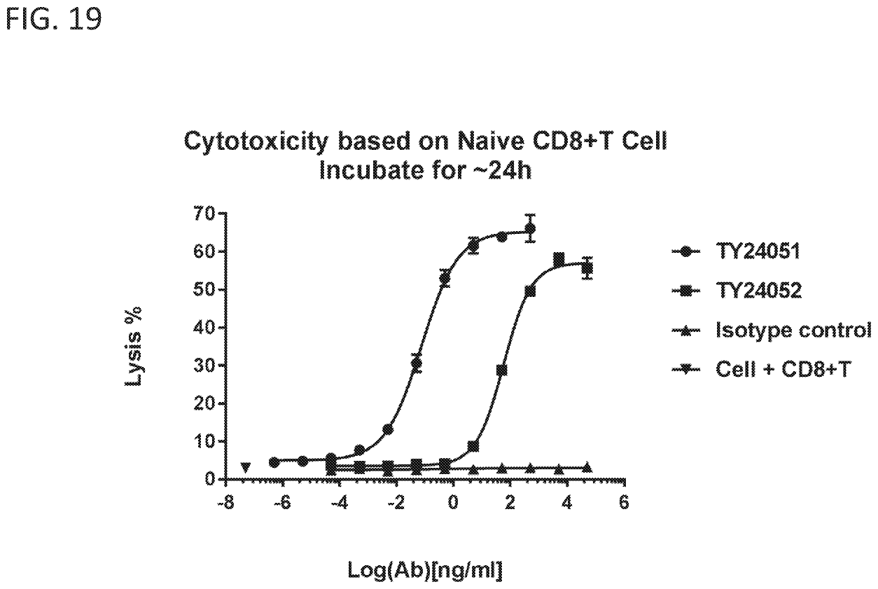

To compare functional activity between TY24051 and TY24052, the antibodies were expressed, purified and evaluated for antigen-dependent bispecific antibody-mediated tumor cell killing activity (FIG. 19). For in vitro cytotoxicity assays, näive human pan-T-cells were isolated from fresh human blood and mixed with HER2-positive tumor cells (SKOV3) along with increasing amounts of bispecific antibody for 24 hours (target cells: 1×10⁴ cells/well, E:T=10:1). As show in FIG. 19, dose dependent killing was observed for TY24051 and TY24052, and TY24052 showed about 800-fold increase the EC₅₀, compared with TY24051. No specific killing was observed with isotype control.

What is claimed is:

1. A heterodimeric protein comprising a first polypeptide comprising a first immunoglobulin heavy chain constant domain 3 (CH3 domain) and a second polypeptide comprising a second CH3 domain, wherein:
i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or
ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or
iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and
wherein the amino acid residue numbering is based on EU numbering.

2. The heterodimeric protein of claim 1, wherein:
i) the first CH3 domain comprises N390C substitution and the second CH3 domain comprises S400C substitution, or the first CH3 domain comprises S400C substitution and the second CH3 domain comprises N390C substitution; or
ii) the first CH3 domain comprises K392C substitution and the second CH3 domain comprises V397C substitution, or the first CH3 domain comprises V397C substitution and the second CH3 domain comprises K392C substitution; or
iii) the first CH3 domain comprises K392C substitution and the second CH3 domain comprises S400C substitution, or the first CH3 domain comprises S400C substitution and the second CH3 domain comprises K392C substitution.

3. The heterodimeric protein of claim 1 or claim 2, wherein:
i) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or
ii) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or
iii) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or
a combination of i) and ii), or a combination of i) and iii); and
wherein the amino acid residue numbering is based on EU numbering.

4. The heterodimeric protein of any one of claims 1-3, wherein the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356; and wherein the amino acid residue numbering is based on EU numbering.

5. The heterodimeric protein of claim 3 or claim 4, wherein:
i) the positively charged residue is a lysine (K) residue, and the negatively charged residue is an aspartic acid (D) residue; or
ii) the positively charged residue is a lysine (K) residue, and the negatively charged residue is a glutamic acid (E) residue; or
iii) the positively charged residue is an arginine (R) residue, and the negatively charged residue is an aspartic acid (D) residue; or
iv) the positively charged residue is an arginine (R) residue, and the negatively charged residue is a glutamic acid (E) residue.

6. The heterodimeric protein of claim 5, wherein:
i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or
ii) the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions; or
iii) the first CH3 domain comprises D356K, E357K and S364K substitutions and the second CH3 domain comprises L351D, K370D and K439D substitutions, or the first CH3 domain comprises L351 D, K370D and K439D substitutions and the second CH3 domain comprises D356K, E357K and S364K substitutions.

7. The heterodimeric protein of any one of claims 1-5, wherein:
i) the first CH3 domain further comprises K392D and K409D substitutions and the second CH3 domain further comprises D356K and D399K substitutions, or the first CH3 domain further comprises D356K and D399K substitutions and the second CH3 domain further comprises K392D and K409D substitutions; or
ii) the first CH3 domain further comprises L368D and K370S substitutions and the second CH3 domain further comprises E357Q and S364K substitutions, or the first CH3 domain further comprises E357Q and S364K substitutions and the second CH3 domain further comprises L368D and K370S substitutions; or
iii) the first CH3 domain further comprises L351K and T366K substitutions and the second CH3 domain further comprises L351D and L368E substitutions, or the first CH3 domain further comprises L351D and L368E substitutions and the second CH3 domain further comprises L351K and T366K substitutions; or
(iv) the first CH3 domain further comprises P395K, P396K and V397K substitutions and the second CH3 domain comprises T394D, P395D and P396D substitutions, or the first CH3 domain further comprises T394D, P395D and P3% D substitutions and the second CH3 domain further comprises P395K, P396K and V397K substitutions; or
(v) the first CH3 domain further comprises F405E, Y407E and K409E substitutions and the second CH3 domain comprises F405K and Y407K substitutions, or the first CH3 domain further comprises F405K and Y407K substitutions and the second CH3 domain further comprises F405E, Y407E and K409E substitutions.

8. The heterodimeric protein of claim 6, wherein
i) the first CH3 domain comprises E357K, S364K and N390C substitutions and the second CH3 domain comprises L351D, K370D, and S400C substitutions, or the first CH3 domain comprises L351D, K370D, and S400C substitutions and the second CH3 domain comprises E357K, S364K and N390C substitutions; or
ii) the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions; or
iii) the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions; or
iv) the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions.

9. The heterodimeric protein of any one of claims 1-8, wherein the first CH3 domain and the second CH3 domain further comprise knob-into-hole residues.

10. The heterodimeric protein of claim 9, wherein:
i) the first CH3 domain comprises T336S, L368A and Y407V substitutions and the second CH3 domain comprises T366W substitution, or the first CH3 domain comprises T366W substitution and the second CH3 domain comprises T336S, L368A and Y407V substitutions; or
ii) the first CH3 domain comprises L368V and Y407V substitutions and the second CH3 domain comprises T366W substitution, or the first CH3 domain comprises T366W substitution and the second CH3 domain comprises L368V and Y407V substitutions.

11. A heterodimeric protein comprising a first polypeptide comprising a first CH3 domain and a second polypeptide comprising a second CH3 domain, wherein:
i) the first CH3 domain comprises a positively charged residue at position 357 and the second CH3 domain comprises a negatively charged residue at position 351, or the first CH3 domain comprises a negatively charged residue at position 351 and the second CH3 domain comprises a positively charged residue at position 357; or
ii) the first CH3 domain comprises a positively charged residue at position 411 and the second CH3 domain comprises a negatively charged residue at position 370, or the first CH3 domain comprises a negatively charged residue at position 370 and the second CH3 domain comprises a positively charged residue at position 411; or
iii) the first CH3 domain comprises a positively charged residue at position 364 and the second CH3 domain comprises a negatively charged residue at position 370, or the first CH3 domain comprises a negatively charged residue at position 370 and the second CH3 domain comprises a positively charged residue at position 364; and wherein the amino acid residue numbering is based on EU numbering.

12. The heterodimeric protein of claim 11, wherein the first CH3 domain comprises a charged residue at position 356 and the second CH3 domain comprises a negatively charged residue at position 439, or first CH3 domain comprises a negatively charged residue at position 439 and the second CH3 domain comprises a positively charged residue at position 356; and wherein the amino acid residue numbering is based on EU numbering.

13. The heterodimeric protein of claim 11 or claim 12, wherein:
i) the positively charged residue is a lysine (K) residue, and the negatively charged residue is an aspartic acid (D) residue; or
ii) the positively charged residue is a lysine (K) residue, and the negatively charged residue is a glutamic acid (E) residue; or
iii) the positively charged residue is an arginine (R) residue, and the negatively charged residue is an aspartic acid (D) residue; or
iv) the positively charged residue is an arginine (R) residue, and the negatively charged residue is a glutamic acid (E) residue.

14. The heterodimeric protein of claim 13, wherein:
i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or
ii) the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions; or
iii) the first CH3 domain comprises D356K, E357K and S364K substitutions and the second CH3 domain comprises L351D, K370D and K439D substitutions, or the first CH3 domain comprises L351 D, K370D and K439D substitutions and the second CH3 domain comprises D356K, E357K and S364K substitutions.

15. The heterodimeric protein of any one of claims 11-14, wherein:
i) the first CH3 domain further comprises K392C substitution and the second CH3 domain further comprises D399C substitution, or the first CH3 domain further comprises D399C substitution and the second CH3 domain further comprises K392C substitution; or
ii) the first CH3 domain further comprises Y394C substitution and the second CH3 domain further comprises S354C substitution, or the first CH3 domain further comprises S354C substitution and the second CH3 domain further comprises Y394C substitution; or
iii) the first CH3 domain further comprises D356C substitution and the second CH3 domain further comprises Y349C substitution, or the first CH3 domain further comprises Y349C substitution and the second CH3 domain further comprises D356C substitution.

16. The heterodimeric protein of any one of claims 1-15, wherein the first CH3 domain and the second CH3 domain are human CH3 domains.

17. The heterodimeric protein of any one of claims 1-16, wherein the first polypeptide and the second polypeptide each comprises from the N-terminus to the C-terminus at least a portion of an immunoglobulin hinge region, an immunoglobulin heavy chain constant domain 2 (CH2 domain), and the CH3 domain.

18. The heterodimeric protein of claim 17, wherein the CH2 domains and the CH3 domains form an IgG Fc region.

19. The heterodimeric protein of claim 18, wherein the Fc region is of the human IgG1 subclass.

20. The heterodimeric protein of claim 18, wherein the Fc region is of the human IgG4 subclass.

21. The heterodimeric protein of claim 20, wherein the Fc region further comprises S228P substitution.

22. The heterodimeric protein of any one of claims 18-20, wherein the Fc region further comprises N297A substitution.

23. The heterodimeric protein of any one of claims 1-22, the first polypeptide and the second polypeptide are antibody heavy chains, and wherein the heterodimeric protein further comprises one or more antibody light chains.

24. The heterodimeric protein of claim 23, wherein the heterodimeric protein is a multispecific antibody.

25. The heterodimeric protein of claim 24, further comprising a third polypeptide and a fourth polypeptide, wherein:
(i) the first polypeptide comprises a structure represented by the formula:
VH1-CH1-hinge-CH2-first CH3-L1-scFv1 (Ia);
(ii) the second polypeptide comprises a structure represented by the formula:
VH2-CH1-hinge-CH2-second CH3-L2-scFv2 (IIa);
(iii) the third polypeptide comprises a structure represented by the formula:
VL1-CL (Ib); and
(iv) the fourth polypeptide comprises a structure represented by the formula:
VL2-CL (IIb);
wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
scFv1 is a first single-chain variable fragment;
scFv2 is a second single-chain variable fragment;
CL is an immunoglobulin light chain constant domain;
CH1 is an immunoglobulin heavy chain constant domain 1:
CH2 is an immunoglobulin heavy chain constant domain 2;
hinge is an immunoglobulin hinge region connecting the CH1 and CH2 domains; and
L1 and L2 is each independently a bond or a peptide linker;
wherein VL1 and VH1 associate to form a first Fv that specifically binds to a first target;
wherein VL2 and VH2 associate to form a second Fv that specifically binds to a second target;
wherein scFv1 specifically binds to a third target; and
wherein scFv2 specifically binds to a fourth target.

26. The heterodimeric protein of claim 25, wherein scFv1 and scFv2 are identical.

27. The heterodimeric protein of claim 25 or 26, wherein VL1 and VL2 are identical.

28. The heterodimeric protein of claim 26 or 27, wherein the first Fv specifically binds PDL1, the second Fv specifically binds CD137, and scFv1 and scFv2 specifically bind CTLA-4.

29. The heterodimeric protein of claim 24, further comprising a third polypeptide, wherein:
(i) the first polypeptide comprises a structure represented by the formula:
VH-CH1-hinge-CH2-first CH3 (IIIa);
(ii) the second polypeptide comprises a structure represented by the formula:
scFv-hinge-CH2-second CH3 (IVa); and
(iii) the third polypeptide comprises a structure represented by the formula:
VL-CL (IIIb);
wherein:
VL is an immunoglobulin light chain variable domain;
VH is an immunoglobulin heavy chain variable domain;
scFv is a single-chain variable fragment;
CL is an immunoglobulin light chain constant domain;
CH1 is an immunoglobulin heavy chain constant domain 1;
CH2 is an immunoglobulin heavy chain constant domain 2;
and hinge is an immunoglobulin hinge region connecting the CH1 and CH2 domains;
wherein VL and VH associate to form an Fv that specifically binds to a first target; and
wherein the scFv specifically binds to a second target.

30. The heterodimeric protein of claim 29, wherein the Fv specifically binds CD137 and the scFv specifically binds PDL1.

31. The heterodimeric protein of claim 24, wherein the heterodimeric protein is an activatable antibody, wherein the heterodimeric protein comprises a third polypeptide, and wherein:
(i) the first polypeptide comprises a structure represented by the formula:
VH-CH1-hinge-CH2-first CH3 (Va);
(ii) the second polypeptide comprises a structure represented by the formula:
MM1-CM1-scFv-hinge-CH2-second CH3 (VIa); and
(iii) the third polypeptide comprises a structure represented by the formula:
MM2-CM2-VL-CL (IVb);
wherein:
VL is an immunoglobulin light chain variable domain;
VH is an immunoglobulin heavy chain variable domain;
scFv is a single-chain variable fragment;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
wherein VL and VH associate to form a first Fv that specifically binds to a first target;
wherein the scFv specifically binds to a second target;
wherein MM1 inhibits the binding of the scFv to the first target when CM1 is not cleaved; and
wherein MM2 inhibits the binding of the first Fv to the second target when CM2 is not cleaved.

32. The heterodimeric protein of claim 29 or 31, wherein the first target is a tumor antigen, and the second target is CD3.

33. The heterodimeric protein of claim 31 or 32, wherein MM1 comprises the amino acid sequence of SEQ ID NO: 35.

34. The heterodimeric protein of claim 32 or 33, wherein the first target is HER2.

35. The heterodimeric protein of claim 34, wherein MM2 comprises the amino acid sequence of SEQ ID NO: 36.

36. An activatable antibody comprising: a first polypeptide comprising, from N-terminus to C-terminus, a masking moiety (MM), a cleavable moiety (CM), and a target binding moiety (TBM), wherein the MM comprises the amino acid sequence of SEQ ID NO: 35; wherein the MM inhibits binding of the activatable antibody to human CD3 when the CM is not cleaved; wherein the CM comprises at least a first cleavage site; and wherein:
a) the TBM comprises a VL and the activatable antibody further comprises a second polypeptide comprising a VH;
b) the TBM comprises a VH and the activatable antibody further comprises a second polypeptide comprising a VL:
c) the TBM comprises from the N-terminus to the C-terminus, a VL and a VH; or
d) the TBM comprise from the N-terminus to the C-terminus, a VH and a VL; and
wherein the activatable antibody binds to human CD3 via the VH and VL when the CM is cleaved.

37. An activatable antibody comprising: a first polypeptide comprising, from N-terminus to C-terminus, a masking moiety (MM), a cleavable moiety (CM), and a target binding moiety (TBM), wherein the MM comprises the amino acid sequence of SEQ ID NO: 36; wherein the MM inhibits binding of the activatable antibody to human HER2 when the CM is not cleaved;
wherein the CM comprises at least a first cleavage site; and wherein:
a) the TBM comprises a VL and the activatable antibody further comprises a second polypeptide comprising a VH;
b) the TBM comprises a VH and the activatable antibody further comprises a second polypeptide comprising a VL;
c) the TBM comprises from the N-terminus to the C-terminus, a VL and a VH; or
d) the TBM comprise from the N-terminus to the C-terminus, a VH and a VL; and
wherein the activatable antibody binds to human HER2 via the VH and VL when the CM is cleaved.

38. The activatable antibody of claim 36 or 37, comprising a first polypeptide, a second polypeptide and a third polypeptide, wherein:
(i) the first polypeptide comprises a structure represented by the formula:
VH-CH1-hinge-CH2-first CH3 (Va);
(ii) the second polypeptide comprises a structure represented by the formula:
MM1-CM1-scFv-hinge-CH2-second CH3 (VIa); and
(iii) the third polypeptide comprises a structure represented by the formula:
MM2-CM2-VL-CL (IVb);
wherein:
VL is an immunoglobulin light chain variable domain;
VH is an immunoglobulin heavy chain variable domain;
scFv is a single-chain variable fragment;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
wherein VL and VH associate to form a first Fv that specifically binds to a first target;
wherein the scFv specifically binds to a second target; and wherein MM is MM1 or MM2.

39. The heterodimeric protein of any one of claims 32-35 or the activatable antibody of claim 36 or 38, wherein the first Fv or the TBM comprises a VH comprising an CDR-H1 comprising the amino acid sequence of SEQ ID NO: 61, a CDR-H2 comprising the amino acid sequence of SEQ ID NO: 62, and/or a CDR-H3 comprising the amino acid sequence of SEQ ID NO: 63. In some embodiments, the TBM comprises a VL comprising a CDR-L1 comprising the amino acid sequence of SEQ ID NO: 64, a CDR-L2 comprising the amino acid sequence of SEQ ID NO: 65, and/or a CDR-L3 comprising the amino acid sequence of SEQ ID NO: 66.

40. The heterodimeric protein of claim 34 or 35, or the activatable antibody of any one of claims 37-39, wherein the scFv or the TBM comprises a VH comprising an CDR-H1 comprising the amino acid sequence of SEQ ID NO: 69, a CDR-H2 comprising the amino acid sequence of SEQ ID NO: 70, and/or a CDR-H3 comprising the amino acid sequence of SEQ ID NO: 71. In some embodiments, the TBM comprises a VL comprising a CDR-L1 comprising the amino acid sequence of SEQ ID NO: 72, a CDR-L2 comprising the amino acid sequence of SEQ ID NO: 73, and/or a CDR-L3 comprising the amino acid sequence of SEQ ID NO: 74

41. The heterodimeric protein of any one of claims 31-35 or the activatable antibody of any one of claims 36-40, wherein the activatable antibody comprises an Fc region comprising a first CH3 domain and a second CH3 domain, wherein the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions.

42. One or more nucleic acid(s) encoding the heterodimeric protein of any one of claims 1-35 and 39-41 or the activatable antibody of any one of claims 36-41.

43. A vector comprising the one or more nucleic acid(s) of claim 42.

44. A host cell comprising the one or more nucleic acid(s) of claim 42 or the vector of claim 43.

45. A method for preparing a heterodimeric protein or an activatable antibody, comprising:
(a) culturing the host cell of claim 44 under conditions that allow expression of the one or more nucleic acid(s) or vector; and
(b) recovering the heterodimeric protein or the activatable antibody from the host cell culture.

46. A pharmaceutical composition comprising the heterodimeric protein of 1-35 and 39-41 or the activatable antibody of any one of claims 36-41, and a pharmaceutically acceptable carrier.

47. A method for treating a disease or condition in a subject in need thereof, comprising administering to the subject an effective amount of the pharmaceutical composition of claim 46.

48. The method of claim 47, wherein the disease or condition is cancer.

49. The method of claim 48, wherein the cancer is lung cancer.

50. The method of claim 48, wherein the cancer is ovarian cancer.
